SEQUENCE LISTING (4 sequences)

SEQ ID NO: 1 - DNA, 20 bp, Artificial Sequence
  Description of Artificial Sequence Synthetic primer
cctcccacaagtctaaaatc

SEQ ID NO: 2 - DNA, 19 bp, Artificial Sequence
  Description of Artificial Sequence Synthetic primer
agcaatgccaatctcggct

SEQ ID NO: 3 - DNA, 20 bp, Artificial Sequence
  Description of Artificial Sequence Synthetic primer
agacaaatcgctccaccaac

SEQ ID NO: 4 - DNA, 20 bp, Artificial Sequence
  Description of Artificial Sequence Synthetic primer
gactcaacacgggaaacctc

(19) United States
(12) Patent Application Publication
(10) Pub. No.: US 2009/0239940 A1
(43) Pub. Date: Sep. 24, 2009

(54) TREATING HEART FAILURE AND VENTRICULAR ARRHYTHMIAS

(76) Inventors: Federica Del Monte, Boston, MA (US); Roger J. Hajjar, New York, NY (US); Fabrice Prunier, Angers (FR)

Correspondence Address: Edwards Angell Palmer & Dodge LLP, P.O. Box 55874, Boston, MA, 02205, US

(21) Appl. No.: 12/391,257

(22) Filed: Feb. 23, 2009

Related U.S. Application Data
(63) Continuation-in-part of application No. 10/914,829, filed on Aug. 10, 2004.
Application No. 10/914,829 is a continuation-in-part of application No. 09/789,894, filed on Feb. 21, 2001, now abandoned.
Application No. 09/789,894 is a continuation-in-part of application No. 09/119,092, filed on Jul. 20, 1998, now abandoned.
(60) Provisional application No. 60/053,356, filed on Jul. 22, 1997.
(60) Provisional application No. 61/066,738, filed on Feb. 22, 2008.
(60) Provisional application No. 61/080,076, filed on Jul. 11, 2008.

(51) Int. Cl. A61K 31/7088; A61K 31/7105; A61K 31/711

(52) U.S. Cl. 514/44 R

(57) ABSTRACT

The present invention provides methods and materials for treating heart disorders, including heart failure and arrhythmia, by enhancing SERCA2a activity. Heart cells in a subject can be treated, for example, by introducing, into the heart of the subject, an adeno-associated virus subtype 6 (AAV6) viral delivery system that includes a functional nucleic acid encoding SERCA2a. For example, the functional nucleic acid encodes a non-viral therapeutic protein, thereby treating the subject.

### RELATED APPLICATIONS AND INCORPORATION BY REFERENCE

This application is a continuation-in-part of U.S. patent application Ser. No. 10/914,829, filed Aug. 10, 2004, which is a continuation-in-part of U.S. patent application Ser. No. 09/789,894, filed Feb. 21, 2001, which is a continuation-in-part of U.S. patent application Ser. No. 09/119,092, filed Jul. 20, 1998, and which claims the benefit of previously filed Provisional Application Nos. 60/053,356, filed Jul. 22, 1997, 61/066,738, filed Feb. 22, 2008, and 61/080,076, filed Jul. 11, 2008, all of which are hereby incorporated by reference in their entireties.

Any and all references cited in the text of this patent application, including any U.S. or foreign patents or published patent applications, International patent applications, as well as, any non-patent literature references, including any manufacturer's instructions, are hereby expressly incorporated herein by reference.

### BACKGROUND OF THE INVENTION

Heart disease represents a global term encompassing a wide variety of conditions and diseases affecting the heart and is the leading cause of the death worldwide. According to the Center for Disease Control, about 25 million people are diagnosed with heart disease each year in the United States, with more than half one million people dying each year in the U.S. alone. Heart disorders include heart failure, myocardial ischemia, arrhythmias, myocardial infarction, congestive heart failure, cardiac arrest, and transplant rejections, among other conditions. Of those, heart failure and arrhythmias represent two important types of heart disorders which together, cause a significant portion of heart disorder-linked deaths.

Heart failure is a debilitating disease in which the heart abnormally functions leading to to an inadequate supply of blood to meet the body's needs. Typically, the heart loses propulsive power because the cardiac muscle loses capacity to stretch and contract. Often, the ventricles do not adequately fill with blood between heartbeats and the valves regulating blood flow become leaky, allowing regurgitation or back-flow of blood. The impairment of arterial circulation deprives vital organs of oxygen and nutrients. Fatigue, weakness and the inability to carry out daily tasks may result. Not all heart failure patients suffer debilitating symptoms immediately. Some may live actively for years. Yet, with few exceptions, the disease is relentlessly progressive. As heart failure progresses, it tends to become increasingly difficult to manage. Even the compensatory responses it triggers in the body may themselves eventually complicate the clinical prognosis.

Cardiac arrhythmias are abnormal heart rhythms that typically occur either in the atria or the ventricles. Arrhythmias arising in the atria are called atrial arrhythmias, and these disorders include atrial fibrillation, atrial flutter, and paroxysmal atrial tachycardia (PSVT). Arrhythmias arising in the ventricles, known as ventricular arrhythmias, are a group of disorders having diverse etiologies, including idiopathic ventricular tachycardia, ventricular fibrillation, and Torsade de Pointes (TdP). Arrhythmias can range from incidental, asymptomatic clinical findings to life-threatening abnormalities, and account for a significant percentage heart disease-related deaths in humans.

The regulation of Ca²⁺ concentration and cycling in cardiac myocytes plays an important role in many heart disorders, including heart failure and arrhythmias.

The sarcoplasmic reticulum (SR) is an internal membrane system, which plays a critical role in the regulation of cytosolic Ca²⁺ concentrations and thus, excitation-contraction coupling in muscle. Contraction is mediated through the release of Ca²⁺ from the SR, while relaxation involves the active re-uptake of Ca²⁺ into the SR lumen by a Ca²⁺-ATPase. In cardiac muscle, the SR Ca²⁺-ATPase activity (SERCA2a) is under reversible regulation by phospholamban.

Phospholamban is a small phosphoprotein, about 6,080 daltons in size, which is an integral element of the cardiac SR membrane. Phospholamban is phosphorylated in vivo in response to β-adrenergic agonist stimulation. In the dephosphorylated state, phospholamban inhibits SR Ca²⁺-ATPase activity by decreasing the affinity of the enzyme for Ca²⁺.

As to heart failure, the condition can be attributed at least in part to a number of abnormalities at the cellular level in the various steps of excitation-contraction coupling of the cardiac cells. One of the key abnormalities in both human and experimental heart failure is a defect in SR function, which is associated with abnormal intracellular Ca²⁺ handling. Deficient SR Ca²⁺ uptake during relaxation has been identified in failing hearts from both humans and animal models and has been associated with a decrease in the activity of SR Ca²⁺-ATPase activity and altered Ca²⁺ kinetics.

As to arrhythmias, while the underlying mechanisms of are heterogeneous, common mechanisms are thought to involve changes in the membrane potential, ion transporters and intracellular Ca²⁺ handling. In one aspect, Ca²⁺ overload of the sarcoplasmic reticulum (SR) is thought to generate spontaneous release of Ca²⁺ by the Ryanodyne-Receptors, resulting in a depolarizing inward current mediated by sodium-calcium exchangers. Other aspects of Ca²⁺ cycling may be involved in generating arrhythmias.

As heart disease continues to be a prevalent cause of death, there is a continual interest and need in developing new and improved treatments and methods for diagnosing heart disease conditions, including especially, heart failure and cardiac arrhythmias.

### SUMMARY OF THE INVENTION

In one aspect, the invention features a method of treating a subject, e.g., a subject having heart failure. The method includes introducing into the subject, e.g., into the heart of a subject, a viral delivery system that includes a therapeutic nucleic acid.

In one embodiment, the viral delivery system is an adeno-associated viral delivery system. The adeno-associated virus can be of serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9).

In one embodiment, the subject has or is at risk for heart failure, e.g. a non-ischemic cardiomyopathy, mitral valve regurgitation, ischemic cardiomyopathy, or aortic stenosis or regurgitation. In one embodiment, the subject needs an improved sarcoplasmic reticulum Ca²⁺ uptake in the cardiac muscle.

In one embodiment, the subject is a human. For example, the subject is between ages 18 and 65. In another embodiment, the subject is a non-human animal.

In one embodiment, the nucleic acid of the viral delivery system encodes a protein, e.g., a heart-specific protein or a protein effective in modulating cardiac physiology.

In one embodiment, the expression of the protein encoded by the nucleic acid of the delivery system is sustained, e.g., for at least three months.

In one embodiment, the nucleic acid of the viral delivery system encodes a sarcoplasmic reticulum Ca²⁺ ATPase pump, e.g., SERCA2a pump. The expression of the sarcoplasmic reticulum Ca²⁺ ATPase pump, e.g., SERCA2a pump can be sustained, e.g., for at least one, two, three, four, or six months.

In one embodiment, the nucleic acid can produce an antisense sequence or siRNA that reduces expression of a desired protein.

In one embodiment, treating the subject ameliorates at least one symptom of heart failure.

In one embodiment, the introduction of the viral delivery system is performed without direct manipulation of the coronary vasculature. In one embodiment, at least 1×10⁹, 1×10¹⁰, 1×10¹¹, 1×10¹², 1×10¹³, 1×10¹⁴, 1×10¹⁵, or 1×10¹⁶ genomes of the virus are delivered. In one embodiment, at most 1×10¹⁹ genomes of the virus are delivered.

In one embodiment, the subject is undergoing or has undergone left ventricular assist device implantation.

In one embodiment, the subject is evaluated after introducing the viral delivery system, e.g., by echocardiography or metabolic stress testing.

In one embodiment, the viral delivery system is introduced by an injection, e.g., a direct injection into the heart, e.g., a direct injection into the left ventricle surface.

In one embodiment, the viral delivery system is introduced by a percutaneous injection, e.g., retrograde from the femoral artery retrograde to the coronary arteries.

In one embodiment, introducing the viral delivery system includes restricting blood flow through coronary vessels, e.g., partially or completely, introducing the viral delivery system into the lumen of the coronary artery, and allowing the heart to pump, while the coronary vein outflow of blood is restricted. Restricting blood flow through coronary vessels can be performed, e.g., by inflation of at least one, two, or three angioplasty balloons. Restricting blood flow through coronary vessels can last, e.g., for at least one, two, three, or four minutes. Introduction of the viral delivery system into the coronary artery can be performed, e.g., by an antegrade injection through the lumen of an angioplasty balloon. The restricted coronary vessels can be: the left anterior descending artery (LAD), the distal circumflex artery (LCX), the great coronary vein (GCV), the middle cardiac vein (MCV), or the anterior interventricular vein (AIV). Introduction of the viral delivery system can be performed after ischemic preconditioning of the coronary vessels, e.g., by restricting blood flow by e.g., inflating at least one, two, or three angioplasty balloons. Ischemic preconditioning of the coronary vessels can last for at least one, two, three, or four minutes.

In one embodiment, introducing the viral delivery system includes restricting the aortic flow of blood out of the heart, e.g., partially or completely, introducing the viral delivery system into the lumen of the circulatory system, and allowing the heart to pump, e.g., against a closed system (isovolumically), while the aortic outflow of blood is restricted. Restricting the aortic flow of blood out of the heart can be performed by redirecting blood flow to the coronary arteries, e.g., to the pulmonary artery. Restricting the aortic flow of blood can be accomplished by clamping, e.g., clamping a pulmonary artery. Introducing the viral delivery system can be performed e.g., with the use of a catheter or e.g., by direct injection. Introducing the viral delivery system can be performed by a delivery into the aortic root.

In another aspect, the invention features a viral delivery system, including a nucleic acid encoding a non-viral therapeutic. In one embodiment, the viral delivery system is an adeno-associated viral delivery system. The adeno-associated virus can be of serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9). For example, the viral delivery system is a modified adeno-associated virus or a reconstituted virus or virus-like particle, e.g., that can infect cells, e.g., a myocytes, e.g., a cardiomyocyte.

In one embodiment, the nucleic acid encodes a protein, e.g., a protein effective in modulating cardiac physiology, e.g., a SERCA2a protein.

In one embodiment, the nucleic acid produces an antisense sequence, e.g., a sequence to downregulate a protein effective in modulating cardiac physiology, e.g., a SERCA2a protein.

In one embodiment, the nucleic acid produces an siRNA sequence, e.g., a sequence to downregulate a protein effective in modulating cardiac physiology, e.g., a SERCA2a protein.

In one embodiment, the nucleic acid includes a heart-specific promoter, e.g., a promoter from cardiac troponin T, alpha myosin light chain, or myosin heavy chain promoter.

In one embodiment, the nucleic acid includes at least a functional segment of a cytomegalovirus (CMV) promoter.

In another aspect, the invention features a method of delivering a compound to the heart of a subject. The method includes: restricting the aortic flow of blood out of the heart, (for example, that the blood flow is redirected to the coronary arteries), introducing the desired compound into the lumen of the circulatory system, e.g., into a blood vessel such as an artery (for example, such that said compound flows into the coronary arteries), allowing the heart to pump while the aortic outflow of blood is restricted (e.g., partially or completely restricted), and reestablishing the flow of blood.

In one embodiment, the compound includes a nucleic acid, which directs the expression of a peptide, e.g., a sarcoplasmic reticulum Ca²⁺ ATPase pump or a β-galactosidase. In another embodiment, the compound includes a protein (e.g., a peptide or larger protein).

In one embodiment, the compound includes a virus vector suitable for somatic gene delivery.

In one embodiment, the compound is delivered using an adeno-associated virus, e.g., serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9). The delivery of the adeno-associated virus can include delivery of a number of genomes, e.g., at least 1×10⁹, 1×10¹⁰, 1×10¹¹, 1×10¹², 1×10¹³, 1×10¹⁴, 1×10¹⁵, or 1×10¹⁶ genomes and, e.g., less than 1×10⁹ genomes.

In one embodiment, the subject is a human.

In one embodiment, the subject is a non-human animal.

In one embodiment, the subject has or is at risk for heart failure, e.g. a non-ischemic cardiomyopathy, mitral valve regurgitation, ischemic cardiomyopathy, or aortic stenosis or regurgitation. The method can include other features described herein.

In another aspect, the invention features a method that includes: restricting the flow of blood through coronary vessels via coronary vein blockade; and introducing a compound into the lumen of the coronary artery. For example, the compound is delivered by a viral delivery system, e.g., an adeno-associated virus delivery system (e.g., an AAV6 system). The compound can be any compound described herein, e.g., a compound that includes a nucleic acid sequence encoding SERCA2a.

The method can include allowing the heart to pump, e.g., while the coronary vein drainage is restricted; and reestablishing the flow of blood through coronary vessels, thereby allowing the said compound to flow into and be delivered to heart cells. The method can include a preconditioning step. For example, the method can include ischemic preconditioning in both the left anterior descending artery and the left circumflex artery is performed. The ischemic preconditioning can be for between 30 seconds and three minutes, e.g., about a one-minute ischemic preconditioning.

The subject is, for example, a human, e.g., a human is suffering from heart failure.

The method can include one or more of: obstructing the left anterior descending artery, obstructing the left circumflex artery, obstructing the great coronary vein, and obstructing the anterior interventricular vein. For example, the method includes obstructing the left circumflex artery and the middle cardiac vein.

The coronary vein blockade can comprise an occlusion by an angioplasty balloon. For example, the obstructions comprise partial or complete occlusions by angioplasty balloons.

The compound can be introduced, for example, by a percutaneous antegrade intracoronary transfer comprising an injection through the center lumen of the inflated angioplasty balloon in either artery. For example, blood flow can be restricted for between 30 seconds and 5 minutes, e.g., for about 2 to 4 minutes or about 3 minutes. It is possible to perform ischemic preconditioning in the left anterior descending artery and/or or the left circumflex artery.

The method can further include opening the pericardium. The method can include other features described herein.

In other aspect the invention features a method of delivering a compound to the heart of a subject, e.g., a subject undergoing a device implantation, e.g., a left ventricular assist device implantation. The method includes introducing (e.g., injecting) the compound into at least one site of the left ventricle. The compound can be delivered using a viral delivery system; e.g., the viral delivery system is introduced. For example, the subject is a human, e.g., a human who has heart failure, e.g., non-ischemic cardiomyopathies.

In one embodiment, the method includes: identifying one or more sites in the left ventricle surface; injecting each site with a virus-containing solution (e.g., between 0.01 to 0.4 ml), below the surface, e.g., at about 5 mm below the surface.

The method can further include evaluating the effects of delivering the compound, e.g., evaluating the effect of the treatment on a parameter related to contractility, e.g., sarcoplasmic reticulum Ca²⁺ ATPase pump activity. The evaluation can include echocardiography and/or metabolic stress testing. The method can include tissue harvest at the time of transplantation.

In one aspect, the invention features a method of treating a subject, e.g., by treating a heart cell of the subject. The subject is a human, or a non-human animal. The method includes introducing into a heart cell, e.g., in a heart tissue, or in a heart, in vitro or in vivo, a nucleic acid which results in the expression of SERCA2a. The method allows for improving the condition of a subject having a heart disorder.

In a preferred embodiment, treating the heart cell includes modulating the ratio of phospholamban to SERCA2a in the heart cell.

In a preferred embodiment, the subject, e.g., a human or a non-human animal, is at risk for, or has, a heart disorder, e.g., heart failure, ischemia, arrhythmia, myocardial infarction, congestive heart failure, transplant rejection, abnormal heart contractility, or abnormal Ca²⁺ metabolism.

In one embodiment, the disorder is one characterized by a deficient SR Ca²⁺ uptake, or one characterized by an increased SR Ca²⁺ uptake.

In a preferred embodiment, the heart disorder is heart failure, ischemia, arrhythmia, myocardial infarction, congestive heart failure, transplant rejection, abnormal heart contractility, or abnormal Ca²⁺ metabolism.

In a preferred embodiment, the nucleic acid is introduced into the subject by somatic gene transfer, e.g., by catheter perfusion. In another preferred embodiment, the nucleic acid is introduced into the subject by somatic gene transfer and is not introduced into the germ line of the subject.

In a preferred embodiment, the subject is a human.

In a preferred embodiment, the nucleic acid is introduced in vitro.

In a preferred embodiment, the nucleic acid is introduced in vivo.

In another embodiment, the method further includes evaluating in the subject any of: survival, cardiac metabolism, heart contractility, heart rate, ventricular function, e.g., left ventricular end-diastolic pressure (LVEDP), left ventricular systolic pressure (LVSP), Ca²⁺ metabolism, e.g., intracellular Ca²⁺ concentration, e.g., peak or resting [Ca²⁺]. SR Ca²⁺ ATPase activity, phosphorylation state of phospholamban, force generation, relaxation and pressure of the heart, a force frequency relationship, cardiocyte survival or apoptosis or ion channel activity, e.g., sodium calcium exchange, sodium channel activity, calcium channel activity, sodium potassium ATPase pump activity, activity of myosin heavy chain, troponin I, troponin C, troponin T, tropomyosin, actin, myosin light chain kinase, myosin light chain 1, myosin light chain 2 or myosin light chain 3, IGF-1 receptor, P13 kinase, AKT kinase, sodium-calcium exchanger, calcium channel (L and T), calsequestrin or calreticulin. The evaluation can be performed before, after, or during the treatment.

In another aspect, the invention features a method of treating a subject, e.g., by treating a heart cell of the subject. The subject is a human, or a non-human animal. The method includes introducing into the subject a nucleic acid that decreases phospholamban activity. In one example, the nucleic acid encodes an antisense sequence, which is at least partially complementary to a phospholamban DNA sequence. In another example, the nucleic acid cassette can produce an siRNA.

In a preferred embodiment, the subject is at risk for, or has, a heart disorder, e.g., heart failure, ischemia, arrhythmia, myocardial infarction, congestive heart failure, transplant rejection, abnormal heart contractility, or abnormal Ca²⁺ metabolism.

In a preferred embodiment, the heart disorder is heart failure, ischemia, arrhythmia, myocardial infarction, congestive heart failure, transplant rejection, abnormal heart contractility, or abnormal Ca²⁺ metabolism.

In a preferred embodiment, the nucleic acid is introduced into the subject by somatic gene transfer, e.g., by catheter perfusion. In another preferred embodiment, the nucleic acid is introduced into the subject by somatic gene transfer and is not introduced into the germ line of the subject.

In a preferred embodiment, the subject is a human, e.g., a human who is at risk for, or has, heart failure.

In a preferred embodiment, the nucleic acid is introduced in vitro.

In a preferred embodiment, the nucleic acid is introduced in vivo.

In another embodiment, the method further includes evaluating in the subject any of: survival, cardiac metabolism, heart contractility, heart rate, ventricular function, e.g., left ventricular end-diastolic pressure (LVEDP), left ventricular systolic pressure (LVSP), Ca²⁺ metabolism, e.g., intracellular Ca²⁺ concentration, e.g., peak or resting [Ca²⁺], SR Ca²⁺ ATPase activity, phosphorylation state of phospholamban, force generation, relaxation and pressure of the heart, a force frequency relationship, cardiocyte survival or apoptosis or ion channel activity, e.g., sodium calcium exchange, sodium channel activity, calcium channel activity, sodium potassium ATPase pump activity, activity of myosin heavy chain, troponin I, troponin C, troponin T, tropomyosin, actin, myosin light chain kinase, myosin light chain 1, myosin light chain 2 or myosin light chain 3, IGF-1 receptor, P13 kinase, AKT kinase, sodium-calcium exchanger, calcium channel (L and T), calsequestrin or calreticulin. The evaluation can be performed before, after, or during the treatment.

In another aspect, the invention features a method of treating a subject, e.g., by treating a heart cell of the subject. The subject is a human, or a non-human animal. The method includes introducing into the subject, e.g., the heart of the subject, a first nucleic acid which results in the expression of an antisense nucleic acid which is at least partially complementary to a phospholamban DNA sequence, and introducing into the subject a second nucleic acid which results in the expression of SERCA2a.

In a preferred embodiment, the subject, e.g., a human or a non-human animal, is at risk for, or has, a heart disorder, e.g., heart failure, ischemia, arrhythmia, myocardial infarction, congestive heart failure, transplant rejection, abnormal heart contractility, or abnormal Ca²⁺ metabolism.

In a preferred embodiment, the heart disorder is heart failure, ischemia, arrhythmia, myocardial infarction, congestive heart failure, transplant rejection, abnormal heart contractility, or abnormal Ca²⁺ metabolism.

In a preferred embodiment, the first and second nucleic acids are introduced into the subject by somatic gene transfer, e.g., by catheter perfusion. In another preferred embodiment, the nucleic acids are introduced into the subject by somatic gene transfer and are not introduced into the germ line of the subject.

In a preferred embodiment, the subject is a human.

In a preferred embodiment, the nucleic acids are introduced in vitro.

In a preferred embodiment, the nucleic acids are introduced in vivo.

In another embodiment, the method further includes evaluating in the subject any of: survival, cardiac metabolism, heart contractility, heart rate, ventricular function, e.g., left ventricular end-diastolic pressure (LVEDP), left ventricular systolic pressure (LVSP), Ca²⁺ metabolism, e.g., intracellular Ca²⁺ concentration, e.g., peak or resting [Ca²⁺], SR Ca²⁺ ATPase activity, phosphorylation state of phospholamban, force generation, relaxation and pressure of the heart, a force frequency relationship, cardiocyte survival or apoptosis or ion channel activity, e.g., sodium calcium exchange, sodium channel activity, calcium channel activity, sodium potassium ATPase pump activity, activity of myosin heavy chain, troponin I, troponin C, troponin T, tropomyosin, actin, myosin light chain kinase, myosin light chain 1, myosin light chain 2 or myosin light chain 3, IGF-1 receptor, P13 kinase, AKT kinase, sodium-calcium exchanger, calcium channel (L and T), calsequestrin or calreticulin. The evaluation can be performed before, after, or during the treatment.

In another aspect, the invention features, a method of evaluating a treatment for a heart disorder. The method includes: providing a heart cell, into which has been introduced by somatic gene transfer, a nucleic acid which results in the expression of phospholamban; administering the treatment to the heart cell; and evaluating the effect of the treatment on the heart cell, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, P13 kinase, AKT kinase or members of the caspase family of proteins. Calcium regulatory proteins include phospholamban, SR Ca²⁺ ATPase, sodium-calcium exchanger, calcium channel (L and T), calsequestrin or calreticulin. The experimental animal can be an animal model for a disorder, e.g., a heart disorder.

In preferred embodiments, the treatment is administered in vitro. In preferred embodiments the cell is derived from an experimental animal or a human. In preferred embodiments the cell can be cultured and/or immortalized.

In preferred embodiments, the nucleic acid encodes a phospholamban protein. The phospholamban can be from the same species that the heart cell is from or it can be from a different species. For example, a mouse phospholamban can be expressed in a mouse cell or a human phospholamban can be expressed in a cell from an experimental animal.

In preferred embodiments, the nucleic acid is introduced into the heart cell by way of a vector suitable for somatic gene transfer, e.g., a viral vector, e.g., an adenoviral vector or an adeno-associated vector (e.g., AAV6).

In another aspect, the invention features, a method of evaluating a treatment for a heart disorder. The method includes: providing a heart, into some or all the cells of which has been introduced, by somatic gene transfer, a nucleic acid which results in the expression of phospholamban; administering the treatment to the heart; and evaluating the effect of the treatment on the heart, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, P13 kinase, AKT kinase or members of the caspase family of proteins. Calcium regulatory proteins include phospholamban, SR Ca²⁺ ATPase, sodium-calcium exchanger, calcium channel (L and T), calsequestrin or calreticulin. The experimental animal can be an animal model for a disorder, e.g., a heart disorder.

In preferred embodiments, the treatment is administered in vitro. In preferred embodiments the heart is derived from an experimental animal or a human.

In preferred embodiments, the nucleic acid encodes a phospholamban protein. The phospholamban can be from the same species that the heart is from or it can be from a different species. For example, a mouse phospholamban can be expressed in a mouse heart or a human phospholamban can be expressed in the heart of an experimental animal. The phospholamban can be delivered to the heart using methods described herein.

In preferred embodiments, the nucleic acid is introduced into the heart by way of a vector suitable for somatic gene transfer, e.g., a viral vector, e.g., an adenoviral vector or an adeno-associated vector (e.g., AAV6).

In another aspect, the invention features, a method of evaluating a treatment for a heart disorder. The method includes: providing heart tissue into some or all of the cells of which has been introduced, by somatic gene transfer, a nucleic acid which results in the expression of phospholamban; administering the treatment to the heart tissue; and evaluating the effect of the treatment on the heart tissue, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, P13 kinase, AKT kinase or members of the caspase family of proteins. Calcium regulatory proteins include phospholamban, SR Ca²⁺ ATPase, sodium-calcium exchanger, calcium channel (L and T), calsequestrin or calreticulin. The experimental animal can be an animal model for a disorder, e.g., a heart disorder.

In preferred embodiments, the treatment is administered in vitro. In preferred embodiments the heart tissue is derived from an experimental animal or a human.

In preferred embodiments, the nucleic acid encodes a phospholamban protein. The phospholamban can be from the same species that the heart tissue is from or it can be from a different species. For example, a mouse phospholamban can be expressed in a mouse heart tissue or a human phospholamban can be expressed in heart tissue from an experimental animal.

In preferred embodiments, the nucleic acid is introduced into the heart tissue by way of a vector suitable for somatic gene transfer, e.g., a viral vector, e.g., an adenoviral vector or an adeno-associated vector (e.g., AAV6).

In another aspect, the invention features, a method of evaluating a treatment for a heart disorder. The method includes: providing a first and a second heart cell, into each of which has been introduced, by somatic gene transfer, a nucleic acid which results in the expression of phospholamban; administering the treatment to a first heart cell, preferably in vitro; evaluating the effect of the treatment on the first heart cell; administering the treatment to a second heart cell, preferably in vivo; and evaluating the effect of the treatment on the second heart cell, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, P13 kinase, AKT kinase or members of the caspase family of proteins. Calcium regulatory proteins include phospholamban, SR Ca²⁺ ATPase, sodium-calcium exchanger, calcium channel (L and T), calsequestrin or calreticulin. The experimental animal can be an animal model for a disorder, e.g., a heart disorder.

In preferred embodiments, the nucleic acid encodes a phospholamban protein. The phospholamban can be from the same species that the heart cell is from or it can be from a different species. For example, a mouse phospholamban can be expressed in a mouse cell or a human phospholamban can be expressed in a cell from an experimental animal.

In preferred embodiments, the first and second cell can be from the same or different animals, can be from the same or different species, e.g., the first cell can be from a mouse and the second cell can be from a human or both cells can be human. The first and second cell can have the same or different genotypes. In further preferred embodiments the evaluation of the treatment in the first cell can be the same or different from the evaluation of the treatment in the second cell, e.g., the intracellular Ca²⁺ concentration can be measured in the first cell and the SR Ca²⁺-ATPase activity can be measured in the second cell or the intracellular Ca²⁺ concentration can be measured in both cells.

In another aspect, the invention features, a method of evaluating a treatment for a heart disorder. The method includes: providing a first administration of a treatment to a heart cell, into which has been introduced by somatic gene transfer, a nucleic acid which results in the expression of phospholamban; evaluating the effect of the first administration on the heart cell; providing a second administration of a treatment to a heart cell, into which has been introduced by somatic gene transfer, a nucleic acid which results in the expression of phospholamban; and evaluating the effect of the second administration on the heart cell, thereby evaluating a treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, P13 kinase, AKT kinase or members of the caspase family of proteins. Calcium regulatory proteins include phospholamban, SR Ca²⁺ ATPase, sodium-calcium exchanger, calcium channel (L and T), calsequestrin or calreticulin. The experimental animal can be an animal model for a disorder, e.g., a heart disorder.

In preferred embodiments, the nucleic acid encodes a phospholamban protein. The phospholamban can be from the same species that the heart cell is from or it can be from a different species. For example, a mouse phospholamban can be expressed in a mouse cell or a human phospholamban can be expressed in a cell from an experimental animal.

In preferred embodiments the first and second administration can be administered to the same or to different cells. The first and second administration can be administered under the same or different conditions, e.g., the first administration can consist of a relatively low level treatment, e.g., a lower concentration of a substance, and the second administration can consist of a relatively high level treatment, e.g., a higher concentration of a substance or both administrations can consist of the same level treatment.

In another aspect, the invention features, a method of evaluating a treatment for a heart disorder. The method includes: providing a heart cell, into which has been introduced by somatic gene transfer, a nucleic acid which results in the expression of phospholamban; administering the treatment to the heart cell; evaluating the effect of the treatment on the heart cell; providing a heart, into which has been introduced by somatic gene transfer, a nucleic acid which results in the expression of phospholamban; administering the treatment to the heart; and evaluating the effect of the treatment on the heart, thereby evaluating the treatment for a heart disorder.

In preferred embodiments, the method includes evaluating the effect of the treatment on a parameter related to heart function. The parameter, by way of example, can include an assessment of contractility, Ca²⁺ metabolism, e.g., intracellular Ca²⁺ concentration, SR Ca²⁺ ATPase activity, force generation, a force frequency relationship, cardiocyte survival or apoptosis or ion channel activity, e.g., sodium calcium exchange, sodium channel activity, calcium channel activity, or sodium potassium ATPase pump activity.

In preferred embodiments, the treatment is administered in vivo, e.g., to an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, P13 kinase, AKT kinase or members of the caspase family of proteins. Calcium regulatory proteins include phospholamban, SR Ca²⁺ ATPase, sodium-calcium exchanger, calcium channel (L and T), calsequestrin or calreticulin. The experimental animal can be an animal model for a disorder, e.g., a heart disorder.

In preferred embodiments, the nucleic acid encodes a phospholamban protein. The phospholamban can be from the same species that the heart cell and/or the heart is from or it can be from a different species. For example, a mouse phospholamban can be expressed in a mouse heart cell and/or heart or a human phospholamban can be expressed in a heart cell and/or heart from an experimental animal.

In preferred embodiments the treatment can be administered to the heart cell in vitro and to the heart in vivo or the treatment can be administered to the heart cell and to the heart in vitro.

In another aspect, the invention features, a method of delivering a compound to the heart of a subject. The method includes: restricting the aortic flow of blood out of the heart, such that blood flow is re-directed to the coronary arteries; introducing the compound into the lumen of the circulatory system such that it flows into the coronary arteries; allowing the heart to pump while the aortic outflow of blood is restricted, thereby allowing the compound to flow into and be delivered to the heart; and reestablishing the flow of blood to the heart.

In preferred embodiments, the compound includes: a nucleic acid which directs the expression of a peptide, e.g., a phospholamban or a SR Ca²⁺-ATPase and a viral vector suitable for somatic gene delivery, e.g., an adenoviral vector or an adeno-associated vector (e.g., AAV6).

In preferred embodiments, the subject is at risk for a heart disorder, e.g., heart failure, ischemia, arrhythmia, myocardial infarction, congestive heart failure, transplant rejection.

In preferred embodiments, the subject can be a human or an experimental animal. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, P13 kinase, AKT kinase or members of the caspase family of proteins. Calcium regulatory proteins include phospholamban, SR Ca²⁺ ATPase, sodium-calcium exchanger, calcium channel (L and T), calsequestrin or calreticulin. The experimental animal can be an animal model for a disorder, e.g., a heart disorder.

In preferred embodiments, the method further includes restricting blood flow into the left side of the heart, e.g., by restricting the pulmonary circulation through obstruction of the pulmonary artery, so as to lessen dilution of the compound.

In preferred embodiments the method further includes opening the pericardium and introducing the compound, e.g., using a catheter.

In preferred embodiments, the compound is: introduced into the lumen of the aorta, e.g., the aortic root, introduced into the coronary ostia or introduced into the lumen of the heart.

In preferred embodiments, the nucleic acid, which directs the expression of the peptide, is homogeneously overexpressed in the heart of the subject.

In another aspect, the invention features, a heart cell, into which has been introduced by somatic gene transfer, a nucleic acid which results in the expression of phospholamban. The heart cell can be provided as a purified preparation.

In another aspect, the invention features, a heart tissue, into which has been introduced by somatic gene transfer, a nucleic acid which results in the expression of phospholamban. The heart tissue can be provided as a tissue preparation.

In another aspect, the invention features, a heart, into which has been introduced by somatic gene transfer, a nucleic acid which results in the expression of phospholamban. The heart can be provided in a subject or ex vivo, i.e. removed from a subject.

In another aspect, the invention features, a method for treating a subject at risk for a heart disorder. The method includes introducing into somatic heart tissue of the subject, a nucleic acid which encodes phospholamban.

In preferred embodiments, the nucleic acid is introduced using the methods described herein.

In preferred embodiments, the phospholamban can be from the same species as the subject or it can be from a different species. For example, a human phospholamban can be introduced into a human heart or a human phospholamban can be can be introduced into the heart of an experimental animal.

In preferred embodiments, the nucleic acid is introduced into the heart by way of a vector suitable for somatic gene transfer, e.g., a viral vector, e.g., an adenoviral vector or an adeno-associated vector (e.g., AAV6).

In certain other aspects, the present invention provides methods for treating and/or preventing cardiac arrhythmias in a subject in need, comprising enhancing the function of SERCA2a.

In still another aspect, the present invention provides a method for treating arrhythmia in a subject with ischemic heart disease, comprising enhancing the function of SERCA2a.

In yet another aspect, the present invention provides a method for protecting against death or reducing injury caused by reperfusion-induced arrhythmia in a subject with acute myocardial ischemia, comprising the step of enhancing the function of SERCA2a.

In a further aspect, the invention provides a method for reducing risk of death or injury due to arrhythmia in a subject with ischemic heart disease comprising:

occluding a coronary artery and a coronary vein with a first and second angioplasty balloon, respectively, thereby restricting the flow of blood through the coronary vessels;

injecting through the lumen of the first angioplasty balloon into the lumen of the coronary artery a recombinant expression vector encoding SERCA2a, thereby causing the vector to perfuse the myocardium, wherein the vector thereafter expresses the SERCA2a in the myocardium, thereby reducing risk of death or injury due to arrhythmia in a subject with ischemic heart disease.

In certain embodiments, enhancing the function of SERCA2a is achieved by administering, e.g., by local administration, to the subject a therapeutically effective amount of a recombinant expression vector encoding SERCA2a.

In other embodiments, the SERCA2a expression vector, when used, is locally administered to the heart of the subject by introducing the SERCA2a expression vector into one or more coronary vessels.

In still further embodiments, the SERCA2a expression vector is administered (e.g., locally administered) to the heart of the subject by introducing the SERCA2a expression vector into one or more coronary vessels by percutaneous anterograde myocardial gene transfer or other similar transfer method.

In other embodiments, the step of enhancing the function of SERCA2a is achieved by administering a therapeutically effective amount of an agent that enhances SERCA2a function.

In certain other embodiments, the agent that enhances SERCA2a function is selected from the group consisting of a compound that increases the activity of SERCA2a, a compound that decreases the activity of phospholamban, or an inhibitory RNA of phospholamban.

In other embodiments, the arrhythmia is a reperfusion-induced arrhythmia.

In certain further embodiments, the arrhythmia is a ventricular tachyarrhythmia. The tachyarrhythmia can be further triggered by acute myocardial ischemia. Further still, the tachycardias can be triggered by reperfusion.

In still further embodiments, the arrhythmia is a ventricular tachyarrhythmia which results from a previously formed myocardial infarction scar. The subject can be without active myocardial ischemia.

In other embodiments, the arrhythmia is a tachyarrhythmia or bradyarrythmia.

In still other embodiments, the arrhythmia is of an atrial, junctional, atro-ventricular or ventricular origin.

In another embodiment, the recombinant expression vector is an adenovirus, adeno-associated virus, or lentivirus vector.

In still another embodiment, the recombinant expression vector is AAV-6.

In yet another embodiment, the agent that is administered in a therapeutically effective amount is administered using a stent.

In aspects involving percutaneous anterograde intracoronary gene transfer, certain embodiments include where the coronary artery is the left anterior descending artery (LAD) or the distal circumflex artery (LCX), and coronary vein is the great coronary vein (GCV), the middle cardiac vein (MCV), or the anterior interventricular vein (AIV).

In certain other embodiments, the step of ischemic preconditioning is for a period of about 1 to 4 minutes.

The methods of the invention allow rapid and low cost development of cardiac overexpression models. The methods of the invention also provide ways of examining multiple genes interacting in transgenic models, testing gene therapy approaches and evaluating treatments of cardiac disorders.

Heart failure secondary to systolic dysfunction is a disease of epidemic proportions in the U.S. with over 5 million effected individuals. Heart failure accounts for over one million hospitalizations, 400,000 deaths, and 40 billion dollars in health care expenses each year with 5-year survival being less than 50%. Recent advances in therapy for patients with mild to moderate symptoms have improved symptoms, decreased hospitalizations and lengthened survival. However, heart failure is a progressive disease and most patients eventually develop unremitting end-stage symptoms. Some patients present either at the time of initial diagnosis or during the course of their disease with fulminant heart failure requiring immediate therapeutic intervention. Historically, transplantation has provided the primary treatment for these patients.

Complete recovery of ventricular function after heart failure and/or cardiac arrhythmias—in particular, ventricular arrhythmias—is still elusive. Failing human hearts of most etiologies are characterized by abnormal intracellular Ca²⁺ regulation secondary to a deficiency in the SR Ca²⁺ ATPase (SERCA2a) pumps. Improvement of contractile function in vitro in human isolated cardiomyocytes has been achieved by reconstituting SERCA2a by gene transfer. This target may offer a new modality for the treatment of heart failure in humans. As to heart failure treatments, the results provided herewith showed that a percutaneous, clinically feasible method of gene transfer of SERCA2a in mitral-regurgitation-induced model of heart failure in the swine reverses contractile dysfunction. As to the cardiac arrhythmias, the inventors showed that SERCA2a overexpression reduces the occurrence of ventricular arrhythmias. This disclosure includes clinical applications of SERCA2a gene therapy for ventricular dysfunction.

Other features and advantages of the invention will be apparent from the following detailed description, and from the claims.

### BRIEF DESCRIPTION OF THE DRAWINGS

The following Detailed Description, given by way of example, but not intended to limit the invention to specific embodiments aspects described, may be understood in conjunction with the accompanying drawings, which incorporated herein by reference. Various features and aspects of the present invention will now be described by way of non-limiting examples and with reference to the accompanying drawings, in which:

FIG. 1 is a graph depicting protein levels of SR Ca²⁺-ATPase in uninfected cardiomyocytes (n=8) and in cardiomyocytes infected for 48 hours with 1, 10, and 100 pfu/cell of Ad.RSV.PL.

FIG. 2 is a graph depicting protein levels of phospholamban and SERCA2a in uninfected cardiomyocytes (n=8) and in cardiomyocytes infected for 48 hours with 10 pfu/cell with either Ad.RSV.PL and/or Ad.RSV.SERCA2a. There were no significant differences between the phospholamban protein levels in the group of myocytes infected with Ad.RSV.PL alone at a multiplicity of infection of 10 pfu/cell and the group of myocytes infected with Ad.RSV.PL at a multiplicity of infection of 10 pfu/cell and Ad.RSV.SERCA2a at a multiplicity of infection of 10 pfu/cell (P>2). Similarly, there were no significant differences between the SERCA2a protein levels in the group of myocytes infected with Ad.RSV.SERCA2a alone at a multiplicity of infection of 10 pfu/cell and the group of myocytes infected with Ad.RSV.PL at a multiplicity of infection of 10 pfu/cell and Ad.RSV.SERCA2a at a multiplicity of infection of 10 pfu/cell (P>2).

FIG. 3A is a graph depicting SERCA2a activity as a function of Ca²⁺ in membrane preparations of uninfected cardiomyocytes (.box-solid., n=6), cardiomyocytes infected with 10 pfu/cell of Ad.RSV.PL (.circle-solid., n=6), and cardiomyocytes infected with 10 pfu/cell of Ad.RSV.PL and 10 pfu/cell of Ad.RSV.SERCA2a (s, n=6).

FIG. 3B is a graph depicting the effect of increasing concentrations of cyclopiazonic acid (CPA) on SRECA2 activity at a [Ca²⁺] of 10 μmol/L in membrane preparations of uninfected cardiomyocytes (.box-solid., n=6), cardiomyocytes infected with 10 pfu/cell of Ad.RSV.PL (.circle-solid., n=6), and cardiomyocytes infected with 10 pfu/cell of Ad.RSV.PL and 10 pfu/cell of Ad.RSV.SERCA2a (s, n=6).

FIG. 4A shows intracellular Ca²⁺ transients and shortening in an uninfected cardiomyocyte and in a cardiomyocyte infected for 48 hours with 10 pfu/cell of Ad.RSV. βgal and stimulated at 1 Hz.

FIG. 4B shows intracellular Ca²⁺ transients and shortening in an uninfected cardiomyocyte and in a cardiomyocyte infected for 48 hours with 1, 10, and 100 pfu/cell of Ad.RSV.PL stimulated at 1 Hz. FIG. 4C shows intracellular Ca²⁺ transients and shortening in an uninfected cardiomyocyte, a cardiomyocyte infected with 1-pfu/cell of Ad.RSV.PL, and a cardiomyocyte infected with 10 pfu/cell of Ad.RSV.PL and 10 pfu/cell of Ad.RSV.SERCA2a for 48 hours, stimulated at 1 Hz.

FIG. 5A is a graph showing the mean of the peak of the intracellular Ca²⁺ transients in uninfected cardiomyocytes (n=10), cardiomyocytes infected with 10 pfu/cell of Ad.RSV.PL (n=12), and cardiomyocytes infected with 10 pfu/cell of Ad.RSV.PL and 10 pfu/cell of Ad.RSV.SERCA2a (n=10) for 48 hours and stimulated at 1 Hz.

FIG. 5B is a graph showing the mean of the resting levels of [Ca²⁺] in uninfected cardiomyocytes (n=10), cardiomyocytes infected with 10 pfu/cell of ad.RSV.PL (n=12), and cardiomyocytes infected with 10 pfu/cell of Ad.RSV.PL and 10 pfu/cell of Ad.RSV.SERCA2a (n=10) for 48 hours, stimulated at 1 Hz.

FIG. 5C is a graph showing the mean of the time to 80% relaxation of the intracellular Ca²⁺ transients in uninfected cardiomyocytes (n=10), cardiomyocytes infected with 10 pfu/cell of Ad.RSV.PL (n=12), and cardiomyocytes infected with 10 pfu/cell of Ad.RSV.PL and 10 pfu/cell of Ad.RSV.SERCA2a (n=10) for 48 hours, stimulated at 1 Hz. P<0.05 compared with uninfected cells. P<0.05 compared with Ad.RSV.PL (multiplicity of infection of 10 pfu/cell).

FIG. 6 is a graph showing the effect of increasing concentrations of Isoprotenerol on the time course of the intracellular Ca²⁺ transients in uninfected cardiomyocytes (n=5) and cardiomyocytes infected with 10 pfu/cell of Ad.RSV.PL (n=5), stimulated at 1 Hz.

FIG. 7A is a graph showing the response of intracellular Ca²⁺ transients to increasing frequency of stimulation in an uninfected cardiomyocyte.

FIG. 7B is a graph showing the response of intracellular Ca²⁺ transients to increasing frequency of stimulation in a cardiomyocyte infected with 10 pfu/cell of Ad.RSV.PL. FIG. 7C is a graph showing the response of intracellular Ca²⁺ transients to increasing frequency of stimulation in a cardiomyocyte infected with 10 pfu/cell of Ad.RSV.PL and 10 pfu/cell of Ad.RSV.SERCA2a for 48 hours.

FIG. 8 shows intracavitary pressure tracings from rats 48 hours after cardiac gene transfer with either Ad.EGFP (left) or Ad.PL (right). The pressure tracing of the Ad.PL transduced hearts displays a markedly prolonged relaxation and reduced pressure development.

FIG. 9 is a drawing showing the somatic gene delivery method.

FIG. 10A is a graph demonstrating that infection of neonatal cardiac myocytes with the construct Ad.asPL increased the contraction amplitude and significantly shortened the time course of the contraction.

FIG. 10B is a graph demonstrating that adenovirus-mediated gene transfer of the antisense cDNA for phospholamban results in a modification of intracellular calcium handling.

FIG. 11 is a graph of survival curves for sham operated animals, and failing animals expressing Sarcoplasmic Reticulum Calcium ATPase through gene transfer. Sham, n=14; sham+Ad.bgal-GFP, n=12; sham+Ad.SERCA2a, n=14; failing, n=14; failing+Ad.bgal-GFP, n=12; failing+SERCA2a, n=16.

FIG. 12 is a bar graph of ATPase activity measured vs. [Ca²⁺] in membrane preparations from sham rats infected with Ad.bgal-GFP (n=4), preparations from failing rat hearts infected with Ad.bgal-GFP (n=4) and preparations of failing hearts infected with Ad.SERCA2a (n=4).

FIG. 13. The failing spectrum illustrates that the PCr-to-ATP ratio and the PCr and ATP contents in the failing heart are lower than in the nonfailing sham heart. In the spectrum of the failing+Ad.SERCA2a heart, the PCr-to-ATP ratio is restored towards normal.

FIG. 14. Left ventricular volumes measured using piezoelectric crystals placed on the surface of the left ventricle in open chested animals. Note the increase in left ventricular volume in failing hearts which is restored towards normal following gene transfer of SERCA2a.

FIG. 15. Six serotypes of AAV, each carrying a beta galactosidase gene under a CMV promoter, were used to inject rat hearts. The graph shows the expression of 5-bromo-4-chloro-3-indolyl α-D-galactopyranoside (X-gal) in rat ventricles at the various time intervals after the injections. AAV6 conferred the fastest, the most specific, and the most efficient gene expression in the heart.

FIG. 16. (A) and (B) show the one-minute ischemic preconditioning (the first vascular blockade) of a pig's heart prior to AAV delivery. The catheter in the AIV and the inflation of the balloon in the proximal LAD are shown. (C) shows a catheter in the AIV of a pig during a 3-minute balloon inflation (the second vascular blockade) in proximal LAD, while the viral vector is injected. (D) shows a catheter in the MCV during a 3-minute balloon inflation (the second vascular blockade) in proximal LCX, while the viral vector is injected.

FIG. 17. AAV6, carrying a beta galactosidase gene under a CMV promoter, was transferred into pig hearts. The Figure shows myocardial sections obtained from such hearts twelve weeks after the gene transfer and stained for X-gal expression. Extensive transfer of beta galactosidase throughout the myocardium is shown. RV=right ventricle; LV=left ventricle.

FIG. 18. Provides a flow chart of Example 17 described below.

FIG. 19. Representative EKG tracings of the types of ventricular arrhythmias from the Holter recordings described in Example 17. A: baseline recording: B: Recording during balloon occlusion; C: Ventricular tachycardia; D: Ventricular fibrillation; E: Idioventricular accelerated rhythm. Abbreviations: VT=ventricular tachycardia, VF=ventricular fibrillation, I/R=ischemia-reperfusion.

FIG. 20. Provides images showing the distribution of the IRDYE 786 and the fluorescent beads as a representation of the distribution of the viral particles using the percutaneous anterograde myocardial gene transfer (PAMGT) method described in Example 17. A solution with red fluorescent beads mixed to fluorescent dye (NIR786 dye, 2 mg/m⁷) is injected in place of the viral solution during PAMGT in the 3 coronary arteries. A, B, C Front view of the entire heart: D, E, F: Heart sections. Left panels: Direct light; Middle panels: Red fluorescent beads distribution; Right panels: IRDYE 786 distribution.

FIG. 21. Depicts the incidence of ventricular arrhythmias in the model of ischemia-reperfusion during ischemia, i.e. the 30 min balloon occlusion (upper panel) and early reperfusion, i.e. the first 10 minutes after balloon deflation (lower panel).

Abbreviations: VT=ventricular tachycardia, VF=ventricular fibrillation, β-gal=pigs receiving β-galactosidase gene, SERCA2a=pigs receiving SERCA2a gene, I/R—ischemia-reperfusion.

FIG. 22. Incidence of ventricular arrhythmias during the follow-up in pigs undergoing I/R injury or PO. A) Effect of SERCA2a overexpression vs. control (β-galactosidase) in the model of I/R showing how SERCA2a protects from sustained and non-sustained ventricular tachycardia. FU=follow-up period in this model corresponds to the late reperfusion, i.e. the period of time from the eleventh minute after balloon deflation to the end of Holter recording. B) In the model of PO SERCA2a failed to protect from tachyarrhythmia. FU=follow-up period in this model corresponds to the period of time from the occurrence of ST elevation to the end of HOLTER recording. Abbreviations: VT=ventricular tachycardia, VF=ventricular fibrillation, I/R=ischemia-reperfusion model. Coil=permanent occlusion model. (β-gal=pigs receiving β-galactosidase gene, SERCA2a—pigs receiving SERCA2a gene.

FIG. 23. Detection of (β-gal gene expression by staining of the ventricular walls of the heart showing that the distribution of the virus as imaged through the IR-Dye and the fluorescent beads correspond to the distribution of viral infection as expressed by X-gal staining (blue staining). Abbreviations: AW=anterior wall, IW—inferior wall, LW=lateral wall, PW—posterior wall. SW=septal wall, RV—right ventricle.

FIG. 24 Quantitative detection of the SERCA2a gene expression in the heart by immunoblotting from samples from 4 pigs each group. The IR group treated with Ad.SERCA2a showed statistically significant increase of the content of SERCA2a (p<0.05).

FIG. 25 Quantitative detection of the SERCA2a gene expression in the heart of four pigs each group by qRT-PCR. The UR group treated with Ad.SERCA2a showed statistically significant increase of the content of SERCA2a messenger (p<0.005). (β-gal=pigs receiving β-galactosidase gene, SERCA2a=pigs receiving SERCA2a gene, I/R=ischemia-reperfusion model, PO=permanent occlusion.

FIG. 26. Representative images of the area at risk assessment in ischemia-reperfusion model (left panel) and PO model (right panel). Upper line shows the front views and lower lines the back view from the same heart.

FIG. 27. a) Images or representative sections of heart from PO groups. IR789 dye and fluorescent beads distribution show similar area at risk in pigs receiving β-galactosidase and SERCA2a. TTC staining images show that nearly the whole area at risk was composed by dead tissue (white) in both groups. Abbreviations: β-gal Coil=permanent coronary artery occlusion and β-galactosidase overexpression group, SERCA2a-Coil=permanent coronary artery occlusion and SERCA2a overexpression group. b) Images of representative sections of heart from ischemia-reperfusion groups. IR789 dye and fluorescent beads distribution show similar area at risk in pigs receiving β-galactosidase or SERCA2a. TTC staining shows that SERCA2a reduced the infarcted area (white).

Abbreviations: β-gal IR=ischemia-reperfusion and β-galactosidase overexpression group, SERCA2a-IR=ischemia-reperfusion and SERCA2a overexpression group.

### DETAILED DESCRIPTION

### Definitions

As used herein, the term “treatment” refers to a procedure (e.g., a surgical method) or the administration of a substance, e.g., a compound which is being evaluated for use in the alleviation or prevention of a heart disorder or symptoms thereof. For example, such treatment can be a surgical procedure, or the administration of a therapeutic agent such as a drug, a peptide, an antibody, an ionophore and the like.

As used herein, the term “heart disorder” refers to a structural or functional abnormality of the heart that impairs its normal functioning. For example, the heart disorder can be heart failure, ischemia, myocardial infarction, congestive heart failure, arrhythmia, transplant rejection and the like. The term includes disorders characterized by abnormalities of contraction, abnormalities in Ca²⁺ metabolism, and disorders characterized by arrhythmia. In a particular embodiment, the heart disorder is an arrhythmia. In another particular embodiment, the heart disorder is an arrhythmia, e.g., ventricular arrhythmia.

As used herein, the term “heart cell” refers to a cell which can be: (a) part of a heart present in a subject, (b) part of a heart which is maintained in vitro, (c) part of a heart tissue, or (d) a cell which is isolated from the heart of a subject. For example, the cell can be a cardiac myocyte.

As used herein, the term “heart” refers to a heart present in a subject or to a heart which is maintained outside a subject.

As used herein, the term “heart tissue” refers to tissue which is derived from the heart of a subject.

As used herein, the term “somatic gene transfer” refers to the transfer of genes into a somatic cell as opposed to transferring genes into the germ line.

As used herein, the term “compound” refers to a compound, which can be delivered effectively to the heart of a subject using the methods of the invention. Such compounds can include, for example, a gene, a drug, an antibiotic, an enzyme, a chemical compound, a mixture of chemical compounds or a biological macromolecule. As used herein, the term “agent” may be used interchangeably with “compound,” and thus, also may refer to a gene, a drug, an antibiotic, an enzyme, a chemical compound or a mixture thereof. As used herein, the term “subject” refers to an experimental animal, e.g., a rat or a mouse, a domestic animal, e.g., a dog, cow, sheep, pig or horse, a non-human primate, e.g., a monkey and in the case of therapeutic methods, humans. However, it is noted that human cells, tissue or hearts can be used in vitro evaluations. A subject can suffer from a heart disorder, such as heart failure, ischemia, myocardial infarction, congestive heart failure, arrhythmia, transplant rejection and the like. The experimental animal can be an animal in which a gene related to cardiac structure or function is misexpressed. Misexpression can be achieved by methods known in the art, for example, by transgenesis, including the creation of knockout animals, or by classic breeding experiments or manipulation. The misexpressed gene can be a gene encoding a sarcomeric protein, a gene encoding a protein which conditions cardiocyte survival or apoptosis, or a gene encoding a calcium regulatory protein. Sarcomeric proteins include myosin heavy chain, troponin I, troponin C, troponin T, tropomyosin, actin, myosin light chain kinase, myosin light chain 1, myosin light chain 2 or myosin light chain 3. Proteins which modify cardiocyte survival or apoptosis include IGF-1 receptor, P13 kinase, AKT kinase or members of the caspase family of proteins. Calcium regulatory proteins include phospholamban, SR Ca²⁺ ATPase, sodium-calcium exchanger, calcium channel (L and T), calsequestrin or calreticulin. The experimental animal can be an animal model for a heart disorder, such as a hypertensive mouse or rat.

In preferred embodiments, the subject can be a human, an experimental animal, e.g., a rat or a mouse, a domestic animal, e.g., a dog, cow, sheep, pig or horse, or a non-human primate, e.g., a monkey. The subject can be suffering from a cardiac disorder, such as heart failure, ischemia, myocardial infarction, congestive heart failure, arrhythmia, transplant rejection and the like. In a preferred embodiment, the subject is suffering from heart failure. In another particular embodiment, the subject is suffering from arrhythmia. As used herein, the term “misexpression” refers to a non-wild type pattern of gene expression. It includes: expression at non-wild type levels, i.e., over- or underexpression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus. Misexpression includes any expression from a transgenic nucleic acid.

As used herein, the term “restricting the aortic flow of blood out of the heart” refers to substantially blocking the flow of blood into the distal aorta and its branches. For example, at least 50% of the blood flowing out of the heart is restricted, preferably 75% and more preferably 80, 90, or 100% of the blood is restricted from flowing out of the heart. The blood flow can be restricted by obstructing the aorta and the pulmonary artery, e.g., with clamps.

As used, herein, the term “introducing” refers to a process by which a compound can be placed into a chamber or the lumen of the heart of a subject. For example, the pericardium can be opened and the compound can be injected into the heart, e.g., using a syringe and a catheter. The compound can be: introduced into the lumen of the aorta, e.g., the aortic root, introduced into the coronary ostia or introduced into the lumen of the heart.

As used herein, the terms “homogeneous fashion” and “homogeneously overexpressing” are satisfied if one or more of the following requirements are met: (a) the compound contacts at least 10%, preferably 20, 20, 40, 50, 60, 70, 80, 90 or 100% of the cells of the heart and (b) at least 10%, preferably 20, 20, 40, 50, 60, 70, 80, 90 or 100% of the heart cells take up the compound.

As used herein, the term “purified preparation” refers to a preparation in which at least 50, preferably 60, 70, 80, 90 or 100% of the cells are heart cells into which phospholamban has been introduced by somatic gene transfer.

The term “obtaining,” as in “obtaining” an compound or agent that can be used in treating a heart disorder, including heart failure or arrhythmia, by any means, including purchasing of same.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

As used herein, the term “inotrope” is an agent that increases the contractility capacity of a muscular contraction.

As used herein, the term “arrhythmia,” also know “cardiac arrhythmia,” refers to a heterogenous group of conditions affecting the electrical behavior of the heart, e.g., a heart beat that is too fast (“tachycardias”), too slow (“bradycardias”) or of irregular pattern. It will be appreciated that arrhythmias may be classified based on their rate (normal, tachycardia, bradycardia), their mechanism (automaticity, re-entry, fibrillation), or by their site of origin (atrial, ventricular, junctional, atrio-ventricular).

As used herein, the term “ischemic heart disease” refers to a heart disorder characterized by reduced blood supply to the myocardium due to a restriction in blood supply caused by various factors, such as, coronary artery disease (atherosclerosis).

As used herein, the term “enhancing the function of SERCA2a” contemplates any means by which to increase the activity of SERCA2a, whether it be, for example, by increased gene expression, increased SERCA2a enzymatic activity, or decreased inhibition of SERCA2a enzymatic activity or other means.

As used herein, the term “locally administering” contemplates the delivery of an agent of the invention (e.g., a SERCA2a-encoding expression vector or a compound that activates SERCA2a activity) directly to the heart, such as by direct injection by syringe into the myocardium, or by release of the agent into a lumen of a coronary vessel that feeds the myocardium using a intraluminal delivery means, such as an angioplasty balloon catheter. “Local administration” contrasts with “systemic administration,” which delivers an agent by ingestion or by needle into a lumen of the circulatory system such that the agent arrives at the heart via blood flow to the heart.

As used herein, the term “inhibitory RNA”, e.g., an inhibitory RNA of phospholamban, refers to a siRNA (small interfering RNA capable of triggering RNA interference) or microRNA or antisense RNA or other inhibitory RNA species which is capable of detecting and binding to the messenger RNA encoding phospholamban or the DNA encoding phospholamban to interfere and block transcription and/or translation or target the mRNA of phospholamban for destruction (e.g., RNA interference).

As used herein, the term “reperfusion-induced arrhythmia” is that type of arrhythmia which becomes triggered during the reperfusion phase of an ischemic event. An “ischemic event” refers to any physiologic condition leading to ischemia of the myocardium which once release or mitigated leads into a reperfusion, i.e., a restoration of the flow of blood.

As used herein, the term “myocardial infarction scar” refers to that portion of the myocardium which previously, as a result of some degree of ischemia, has resulted in some cell death and a concomitant build-up of scar-forming collagen.

The term “percutaneous anterograde myocardial gene transfer” refers to a procedure used to deliver an agent (e.g., a SERCA2a-encoding expression vector) by local administration into the lumen of the a coronary vessel by using angioplasty balloon catheters to restrict the blood flow in a coronary artery and vein while delivering the agent via the angioplasty balloon, the procedure of which is described in more detail herein, for instance, in Examples 14-17.

Other definitions appear in context throughout this disclosure.

SERCA2a/Phospholamban and Heart Disorder

Somatic gene transfer, e.g., adenoviral or adeno-associated viral gene transfer, is particularly effective in mammalian myocardium both in vivo and in vitro. Gene transfer techniques can be used to ameliorate at least one symptom of a subject having heart failure or other heart disorder associated with altered SR Ca²⁺ physiology. In particular, adeno-associated viral systems (e.g., AAV6) can be used to provide a nucleic acids to heart cells in a subject. The viral system can deliver a gene encoding a protein that modulates heart cell activity, e.g., a gene encoding SERCA2a or other transmembrane regulator.

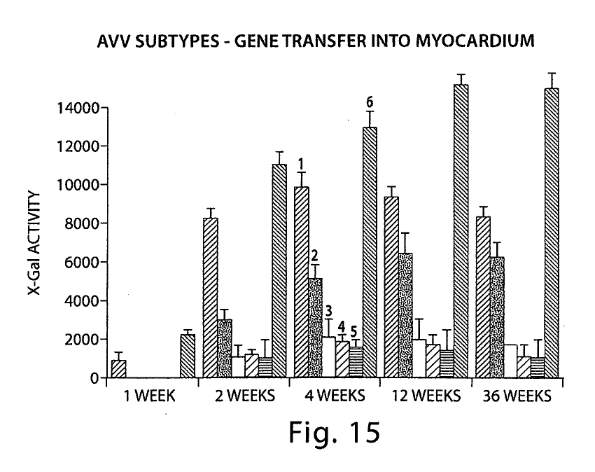

We observed that, with respect to heart cells, AAV6 conferred the fastest gene expression, as well as the most specific and efficient expression in the heart, compared to other AAVs. (See Example 13 and FIG. 15.) Such other AAVs, however, may be useful for other applications, e.g., ones in which a different level or course of expression is desired in the heart.

In addition, adenoviral gene transfer of SERCA2a is both dose dependent and time dependent in rat neonatal cardiomyocytes. An adenovirus encoding phospholamban under the RSV promoter, provided a 4-fold increase in phospholamban, which was also dose dependent. The smaller size of phospholamban compared with SERCA2a (6 kD in its monomer form compared with 110 kD) may explain, at least in part, the more effective protein expression by Ad.RSV.PL than by Ad.RSV.SERCA2a under similar conditions. Nevertheless, using these recombinant adenoviruses, significant overexpression of phospholamban and SERCA2a was achieved, individually and in combination. Co-infection with both Ad.RSV.PL and Ad.RSV.SERCA2a mediated overexpression of both SERCA2a and phospholamban that was the same as the expression from infection with either Ad.RSV.PL or Ad.RSV.SERCA2a alone. The ability to simultaneously manipulate expression of multiple proteins in the context of primary myocytes is an advantage of somatic gene transfer for the study of interacting components of complex systems.

The expression of phospholamban relative to SERCA2a is altered in a number of disease states. In hypothyroidism phospholamban levels are increased, whereas in hyperthyroidism phospholamban levels are decreased. An increased ratio of phospholamban to SERCA2a is an important characteristic of both human and experimental heart failure. Both experimental and human heart failure are characterized by a prolonged Ca²⁺ transient and impaired relaxation. Increasing levels of phospholamban relative to SERCA2a significantly altered intracellular Ca²⁺ handling in the isolated cardiomyocytes by prolonging the relaxation phase of the Ca²⁺ transient, decreasing Ca²⁺ release, and increasing resting Ca²⁺. These results show that altering the relative ratio of phospholamban to SERVA2a can account for the abnormalities in Ca²⁺ handling observed in failing ventricular myocardium. In addition, overexpressing SERCA2a can largely “rescue” the phenotype created by increasing the phospholamban-to-SERCA2a ratio. Restoring the normal phospholamban-to-SERCA2a ratio through somatic gene transfer can correct the abnormalities of Ca²⁺ handling and contraction seen in failing hearts.

### Evaluation of Treatment

A treatment can be evaluated by assessing the effect of the treatment on a parameter related to contractility. For example, SR Ca²⁺ ATPase activity or intracellular Ca²⁺ concentration can be measured, using the methods described above. Furthermore, force generation by hearts or heart tissue can be measured using methods described in Strauss et al., Am. J. Physiol., 262:1437-45, 1992, the contents of which are incorporated herein by reference.

In many drug screening programs which test libraries of therapeutic agents and natural extracts, high throughput assays are desirable in order to maximize the number of therapeutic agents surveyed in a given period of time. Assays which are performed in cell-free systems, such as may be derived with cardiac muscle cell extracts, are preferred as “primary” screens in that they can be generated to permit rapid development and relatively easy detection of the parameter being measured, e.g., the intracellular levels of Ca²⁺, which is mediated by a test therapeutic agent. Moreover, the effects of cellular toxicity and/or bioavailability of the test therapeutic agent can be generally ignored in the in vitro system, the assay instead being focused primarily on the effect of the therapeutic agent on the parameter being measured, e.g., the intracellular levels of Ca²⁺. It is often desirable to screen candidate treatments in two stages, wherein the first stage is performed in vitro, and the second stage is performed in vivo.

The efficacy of a test therapeutic agent can be assessed by generating dose response curves from data obtained using various concentrations of the test therapeutic agent. Moreover, a control assay can also be performed to provide a baseline for comparison. In the control assay, the heart cell is incubated in the absence of a test agent.

### Propagation of Heart Cells

A heart cell culture can be obtained by allowing heart cells to migrate out of fragments of heart tissue adhering to a suitable substrate (e.g., a culture dish) or by disaggregating the tissue, e.g., mechanically or enzymatically to produce a suspension of heart cells. For example, the enzymes trypsin, collagenase, elastase, hyaluronidase, DNase, pronase, dispase, or various combinations thereof can be used. Trypsin and pronase give the most complete disaggregation but may damage the cells. Collagenase and dispase give a less complete dissagregation but are less harmful. Methods for isolating tissue (e.g., heart tissue) and the disaggregation of tissue to obtain cells (e.g., heart cells) are described in Freshney R. I., Culture of Animal Cells, A Manual of Basic Technique, Third Edition, 1994, the contents of which are incorporated herein by reference.

### Viral Vectors Suitable for Somatic Gene Transfer

Expression vectors, suitable for somatic gene transfer, can be used to express the compound, e.g., a SERCA2a gene or a phospholamban gene. Examples of such vectors include replication defective retroviral vectors, adenoviral vectors and adeno-associated viral vectors (AAVs).

Adenoviral vectors suitable for use by the methods of the invention include (Ad.RSV.lacZ), which includes the Rous sarcoma virus promoter and the lacZ reporter gene as well as (Ad.CMV.lacZ), which includes the cytomegalovirus promoter and the lacZ reporter gene. Methods for the preparation and use of viral vectors are described in WO 96/13597, WO 96/33281, WO 97/15679, and Trapnell et al., Curr. Opin. Biotechnol. 5 (6):617-625, 1994, the contents of which are incorporated herein by reference.

Adeno-associated virus is a nonpathogenic human parvovirus, capable of site-specific integration into chromosome 19. Fisher et al., Nature Medicine 3 (3):306-312, 1997. Replication of the virus, however, requires a helper virus, such as an adenovirus. Fisher et al., Nature Medicine 3 (3):306-312, 1997. An AAV coding region can be replaced with nonviral genes, and the modified virus can be used to infect both dividing and non-dividing cells. Xiao et al., Jo. Virol. 70 (11): 8098-8108, 1996; Kaplitt et al., Ann. Thorac. Surg. 62: 1669-1676, 1996. Exemplary methods for the preparation and use of AAVs are described in Fisher et al., Nature Medicine 3 (3):306-312, 1997; Xiao et al., Jo. Virol. 70 (11): 8098-8108, 1996; Kaplitt et al., Ann. Thorac. Surg. 62: 1669-1676, 1996, the contents of which are incorporated herein by reference.

AAV6 is specific and confers fast expression in the heart. Example 14 demonstrates that gene transfer with AAV6 in the heart of a large animal induces excellent efficiency. Example 15 shows that AAV6.CMV.SERCA2a delivered to a pre-clinical large animal model of heart failure induces improvement in ventricular function and reverses heart failure.

### Expression of Phospholamban

The nucleic acid which results in the overexpression of phospholamban can be derived from the natural phospholamban gene including all the introns and exons, it can be a cDNA molecule derived from the natural gene (Fujji et al., J. Biol. Chem. 266:11669-11675, 1991, the contents of which are incorporated herein by reference) or a chemically synthesized cDNA molecule. The nucleic acid encoding the phospholamban protein can be under the control of the naturally occurring promoter or any other promoter that drives a high level expression of the phospholamban gene.

### Treating Arrhythmia

The present invention achieves improved treatment of cardiac arrhythmias by exposing the heart to an agent that enhances the activity of SERCA2a. The enhanced activity of SERCA2a can be a result of increased gene expression of SERCA2a. The enhanced activity can also be attained through the use of a compound that increases the enzymatic function of SERCA2a. In addition, enhancing the activity of SERCA2a can be achieved by mitigating any negative regulation of the expression or activity of SERCA2a. For example, phospholamban, which inhibits SERCA2a, can be inhibited by small molecule inhibitors or by genetic means at the DNA or RNA level. For example interfering RNA molecules can be utilized to reduce the expression of phospholamban, thereby indirectly increasing the activity of SERCA2a.

The present inventors have discovered that arrhythmias can be treated by improving abnormal Ca²⁺ cycling by targeting SERCA2a activity and/or function.

In one aspect, the invention involves the enhancing of the activity of SERCA2a.

In a particular aspect, the arrhythmia is treated by percutaneous anterograde intracoronary gene transfer, comprising the steps of:

occluding a coronary artery and a coronary vein with a first and second angioplasty balloon, respectively, thereby restricting the flow of blood through the coronary vessels;

injecting through the lumen of the first angioplasty balloon into the lumen of the coronary artery a recombinant expression vector encoding SERCA2a, thereby causing the vector to perfuse the myocardium, wherein the vector thereafter expresses the SERCA2a having a homogenous distribution in the myocardium, thereby reducing risk of death or injury due to arrhythmia in a subject with ischemic heart disease.

The agent of the invention used to enhance SERCA2a activity can include, for example, a DNA expression vector (e.g., an adenovirus or adeno-associated virus encoding the SERCA2a gene) delivered systemically or locally to the heart myocardium, which then is expressed in the heart myocardium, leading to an overexpression of the protein and an concomitant increase in its overall activity. The agent used to enhance SERCA2a activity can also include, for example, a small molecule or compound which interacts or binds to SERCA2a thereby causing its activity to become enhanced.

SERCA2a activity can also be enhance indirectly by targeting any native inhibitor of SERCA2a, such as, for example, phospholamban. Inhibition of phospholamban can occur at the genetic, i.e., by blocking transcription or translation or by targeting its mRNA for destruction using tools such as RNA interference. RNA interference and other RNA inhibitory technologies are well-known and can be referenced in, for example, U.S. Pat. Nos. 7,282,564, 7,056,704, 7,078,196 and 6,506,559, each of which are incorporated herein by reference.

In other aspect, the method of enhancing SERCA2a of the invention can include the co-administration of a known antiarrythmic agent, such as any Class I agent (agents that interfere with the sodium channel), Class II agent (anti-sympathetic nervous system agents, e.g. beta-blockers), Class III agent (agents that affect potassium efflux), Class IV agent (agents that affect calcium channels and the AV node), or Class V agent (agents that work by other or unknown mechanisms). Such known antiarrythmic agents that may be used in the present invention by co-administration with the SERCA2a-enhancing agent of the invention include, but is not limited to, Quinidine, Procainamide, Disopyramide, Lidocaine, Phenyloin, Mexiletine, Flecainide, Propafenone, Moricizine, Propranolol, Esmolol, Timolol, Metoprolol, Atenolol, Amiodarone, Sotalol, Ibutilide, Dofetilide, Verapamil, Diltiazem, Adenosine, and Digoxin.

The following examples which further illustrate the invention should not be construed as limiting.

### EXAMPLES

### 1. Construction of El-Deleted Recombinant Adenovirus Vectors

The construction of Ad.RSV.SERCA2a has been described in detail by Hajjar et al., Circulation, 95: 423-429, 1997, the contents of which are incorporated herein by reference. Ad.RSV.βgal, which carries a nuclear localizing form of β-galactosidase, is described in Dong et al., J. Biol. Chem. 27:29969-77, 1996, the contents of which are incorporated herein by reference. The rabbit phospholamban cDNA is described in Lylton J. MacLennan D. H., J. Biol. Chem., 1988, 263:15024-15031, the contents of which are incorporated herein by reference. Briefly, the phospholamban cDNA was subcloned into the bacterial plasmid vector pAdRSV4, which uses the RSV long terminal repeat as a promoter and the SV40 polyadenylation signal and contains map units with adenovirus sequences from 0 to 1 and from 9 to 16. The position and orientation of the phospholamban cDNA were confirmed by restriction enzyme digestion and by polymerase chain reaction. The plasmid vector containing phospholamban (pAd.RSV-PL) was then cotransfected into 293 cells with PJM17. The homologous recombinants between pAd.RSV.PL and pJM17 contain the phospholamban cDNA substituted for E1. By use of this strategy, independent plaques were isolated, and expression of phospholamban protein was verified by immunostaining. A positive plaque was further plaque-purified, and protein expression was reconfirmed to yield the recombinant adenovirus Ad.RSV.PL. This adenovirus is structurally similar to Ad.RSV.βgal and to Ad.RSV.SERCA2a, described in Dong O. et al., J. Biol. Chem., 1996, 271:29969-29977, 1976. The recombinant viruses were prepared as high-titer stocks by propagation in 293 cells as described in Graham, F. L. et al., Methods in Molecular Biology: Gene Transfer and Expression Protocols, 1991, 109-128, the contents of which are incorporated herein by reference. The titers of stocks used for these studies were as follows: 3.1×10¹⁴ pfu/mL for Ad.RSV.PL 2.6×10¹⁰ pfu/m: for Ad.RSV.SERCA2a, and 2.7×10¹⁴ pfu/mL for Ad.RSV.βgal, with a particle-to-pfu ratio of 40:1, 42:1, and 37:1, respectively.

### 2. Preparation of Neonatal Cardiomyocytes

Spontaneously beating cardiomyocytes were prepared from 1 to 2 day old rats and cultured in P-10 medium (GIBCO, BRL) in the presence of 5% fetal calf serum and 10% horse serum for 3 days as described previously in Kang J. X. et al., Proc. Natl. Acad. Sci. U.S.A., 1995, 92:3097-4001 and Kang J. X. and Leaf A., Euro. J. Pharmacol., 1996, 297:97-106, the contents of which are incorporated herein by reference. Measurements of cell shortening and cytosolic Ca²⁺ were performed on neonatal cardiomyocytes cultured on round, coated, glass coverslips (0.1 mm thickness, 31 mm diameter) in 35 mm culture dishes. Cells were counted using a hemocytometer. Approximately 5×10⁵ cells were plated in each coverslip.

### 3. Adenoviral Infection of Isolated Cells

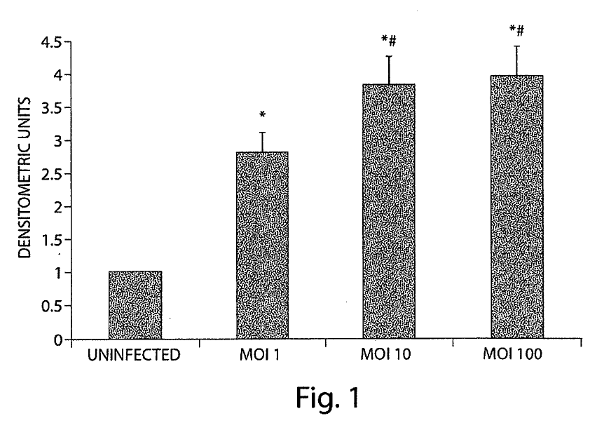

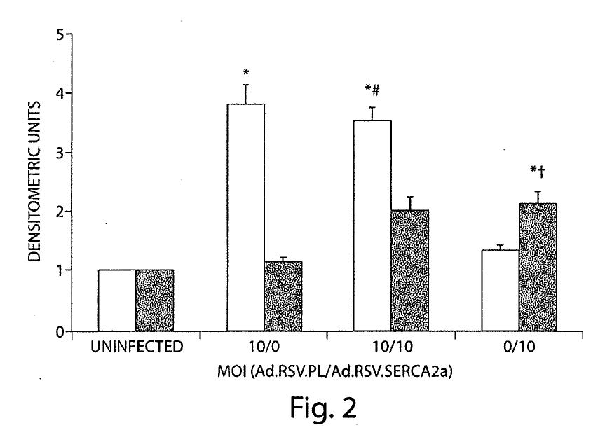

In three different infection experiments with increasing concentrations of Ad.RSV.βgal, the percentages of cells expressing βgal after 48 hours, by histochemical staining in 10 different high-power fields were 98.2% (multiplicity of infection, 1 pfu/cell), 99.1% (multiplicity of infection, 10 pfu/cell), and 100% (multiplicity of infection, 100 pfu/cell). In a similar manner, myocardial cells were infected with three concentrations of Ad.RSV.PL 1.0, 10. and 100 pfu/cell for 48 hours. Infection with either Ad.RSV.βgal, Ad.RSV.PL, or Ad.RSV.SERCA2a did not change the morphology of the cells. For each infection experiment with the adenovirus, one myocyte was used to measure functional parameters. As shown in FIG. 1, there was a 4-fold increase in phospholamban protein levels in a dose-dependent increase in the protein expression of phospholamban between 1 and 10 pfu/cell but no further increases between 10 and 100 pfu/cell. Coinfection of Ad.RSV.SERCA2a with Ad.RSV.PL produced an increase in protein expression of both SERCA2a and phospholamban, as shown by the immunoblot in FIG. 2. There were no significant differences between the phospholamban protein levels in the group of myocytes infected with Ad.RSV.PL alone at a multiplicity of infection of 10 pfu/cell and the group of myocytes infected with Ad.RSV.PL at an multiplicity of infection of 10 pfu/cell and Ad.RSV.SERCA2a at a multiplicity of infection of 10 pfu/cell (P>2). Similarly, there were no significant differences between the SERCA2a protein levels in the group of myocytes infected with Ad.RSV.SERCA2a alone at a multiplicity of infection of 10 pfu/cell and the group of myocytes infected with Ad.RSV.PL at a multiplicity of infection of 10 pfu/cell and Ad.RSV.SERCA2a at an multiplicity of infection of 10 pfu/cell (P>2).

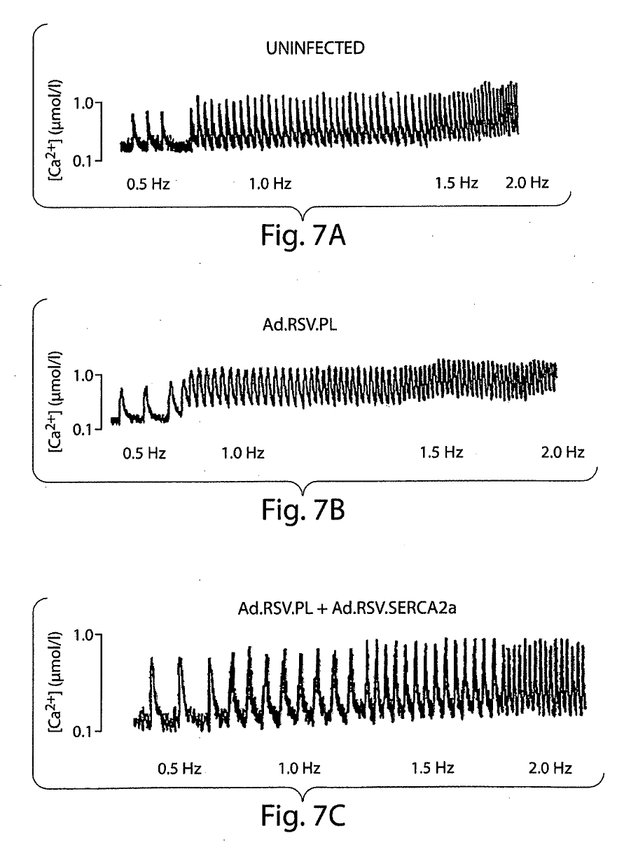

As shown in FIG. 7, cardiomyocytes infected with Ad.RSV.PL (multiplicity of infection of 10 pfu/cell) exhibited a significant increase in resting Ca²⁺ not evident in uninfected cells. Furthermore, coinfection with Ad.RSV.SERCA2a (multiplicity of infection of 10 pfu/cell) restored the frequency response to normal.

The response to increasing stimulation frequencies in mammalian cardiomyocytes is governed by the SR. We have shown that in the uninfected cardiomyocytes, an increase in stimulation frequency did not significantly alter either peak or resting [Ca²⁺]. This response is typical of rat cardiomyocytes that have either a flat response to increasing frequency of stimulation or a decrease in contractile force. However, in cardiomyocytes infected with Ad.RSV.PL, there was a significantly greater increase in resting [Ca²⁺] and a decrease in peak [Ca²⁺]. These results would suggest that diminished SR Ca²⁺ uptake leads to a diminished Ca²⁺ release, which becomes even more accentuated at higher frequencies of stimulation.

### 4. Intracellular Ca²⁺ Measurements and Cell Shortening Detection

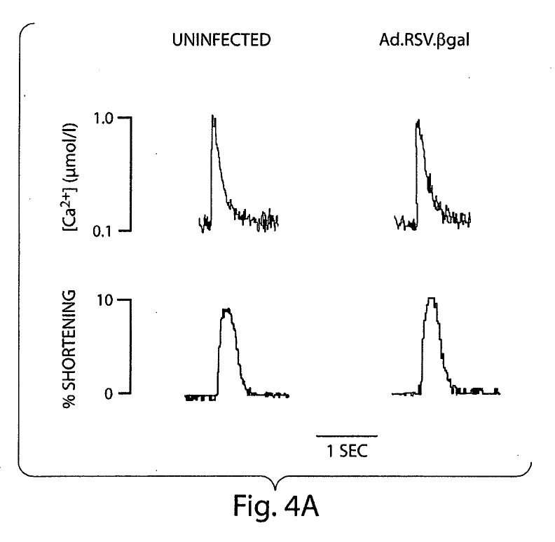

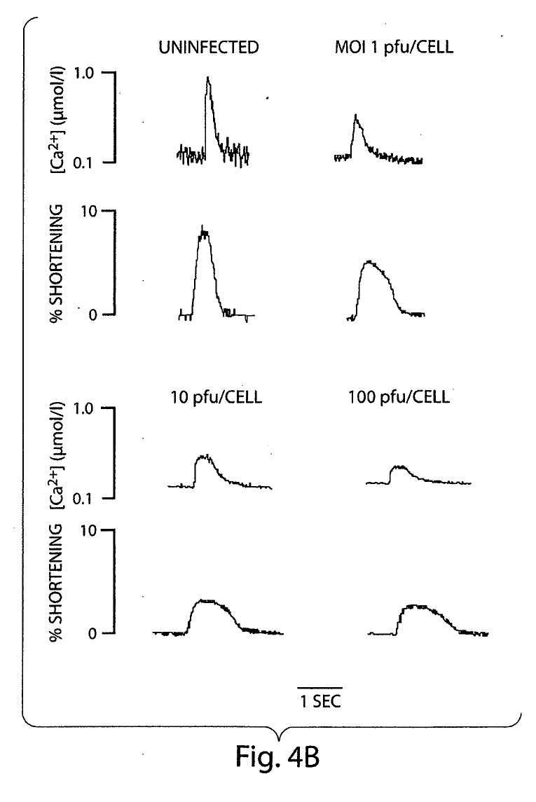

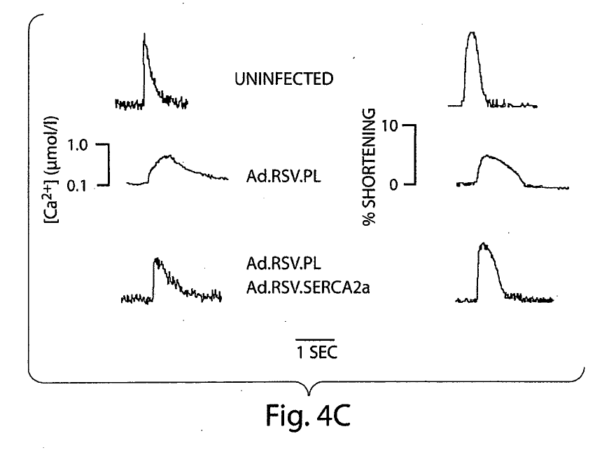

Measurements of intracellular Ca²⁺ and cell shortening were performed as described earlier in Hajjar et al. (1997), Kang et al. (1995) and Kang et al (1996), the contents of which are incorporated herein by reference. Briefly, myocardial cells were loaded with the Ca²⁺ indicator fura 2 by incubating the cells in medium containing 2 μmol/L fura 2-AM (Molecular Probex) for 30 minutes. The cells were then washed with PBS and allowed to equilibrate for 10 minutes in a light-sealed temperature-controlled chamber (32° C.) mounted on a Zeiss Axlovers 10 inverted microscope (Zeiss). The coverslip was superfused with a HEPES-buffered solution at a rate of 20 mL/h. Cells were stimulated at different frequencies (0.1 to 2.0 Hz) using an external stimulator (Grass Instruments). A dual excitation spectrofluorometer (IONOPTIX) was used to record fluorescence emissions (505 nm) elicited from exciting wavelengths of 360 and 380 nm. [Ca²⁺] was calculated according to the following formula: [Ca²⁺]=K_{d}(R−Rₘᵢₙ)/(Rₘₐₓ−R)D, where R is the ratio of fluorescence of the cell at 360 and 380 nm: Rₘᵢₙ and Rₘₐₓ represent the ratios of fura 2 fluorescence in the presence of saturating amounts of Ca²⁺ and effectively “zero Ca²⁺ respectively, K_{d} is the dissociation constant of Ca²⁺ from fura 2; and D is the ratio of fluorescence of fura 2 at 380 nm in zero Ca²⁺ and saturating amounts of Ca²⁺. Unless otherwise stated, measurements of peak [Ca²⁺] were made at the end of diastole. High-contrast microspheres attached to the cell surface of the cardiomyocytes were imaged using a charge-coupled device video camera attached to the microscope, and motion along a selected raster line segment who quantified by a video motion detector system (IONOPTIX). As shown in FIG. 4A, cardiomyocytes infected with Ad.RSV.βgal did not affect the Ca²⁺ transient or shortening compared with control uninfected cardiomyocytes. As depicted in FIG. 4B, the Ca²⁺ transient and shortening were significantly altered with increasing concentrations of Ad.RSV.PL (multiplicity of infection of 1, 10, and 100 pfu/cell): observed changes included prolongation of the Ca²⁺ transient and shortening and a decrease in the peak Ca²⁺. These results, summarized in Table 1, show that there was a dose-dependent prolongation of the Ca²⁺ transient and mechanical shortening up to 10 pfu/cell, with no further significant prolongation at 100 pfu/cell, with no further significant prolongation at 100 pfu/cell.

TABLE 1
Physiological Parameters of Cardiomyocytes Overexpressing
Phospholamban

| | | Ad.RSV.PL | | |
|---|---|---|---|---|
| | | MOI = | MOI = | MOI = |
| | Uninfected | 1 pfu/Cell | 10 pfu/Cell | 100 pfu/Cell |
| Time to 80% | 344 ± 26 | 612 ± 38° | 710 ± 58 | 683 ± 50 |
| relaxation of the | | | | |
| (Ca2+) m? | | | | |
| Time to 80% | 387 ± 22 | 544 ± 27° | 780 ± 44 | 798 ± 43 |
| relaxation of | | | | |
| shortening; m? | | | | |
| Peak [Ca2+]; | 967 ± 43 | 798 ± 23° | 630 ± 33 | 590 ± 34 |
| μmol/L | | | | |
| n | 10 | 8 | 12 | 8 |

Table 1.: Baseline echocardiography and hemodynamic data.
Values are mean ± S.D.
β-gal, pigs receiving β-galaetosidase gene; SFRCA2a, pigs receiving SERCA2a gene; Coil, permanent coronary artery occlusion; I/R, isehemia-reperfusion; Sham, no coronary artery occlusion.
AW, anterior wall thickness in diastole (d) and systole (s): LVEDD LV end-diastolic diameter; LVESD, LV end-srolic diameter; SF, shortening fraction; hR, heart rate; SAP, systolic aortic pressure; CO, cardiac output; LVEDP, LV end-diastolic pressure; dP/dt, first derivative of LV pressure.

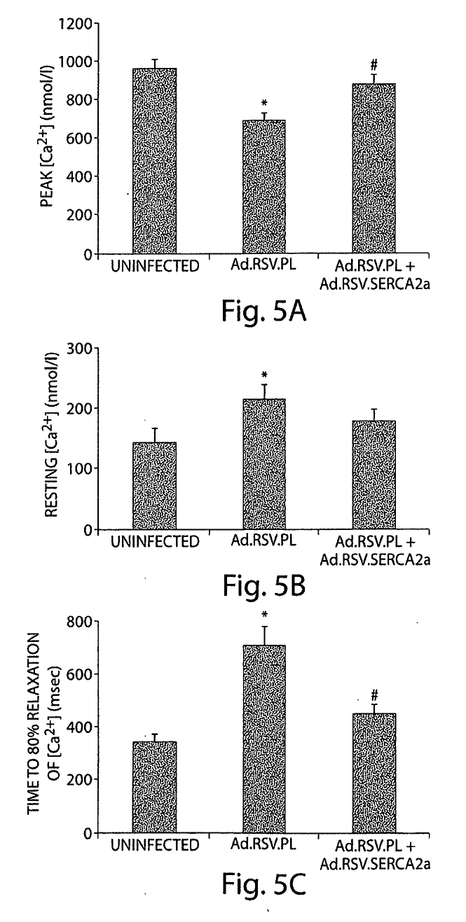

Similarly, peak [Ca²⁺] decreased up to 10 pfu/cell, with no further decrease at 100 pfu/cell. Coinfection with Ad.RSV.SERCA2a (multiplicity of infection of 10 pfu/cell) restored both the Ca²⁺ transient and the shortening to near normal levels, as shown in FIG. 4C. FIG. 5 shows a significant decrease in mean peak [Ca²⁺], a significant increase in mean resting [Ca²⁺], and a significant prolongation of the Ca²⁺ transient in the group of cardiomyocytes infected with Ad.RSV.PL (multiplicity of infection of 10 pfu/cell) compared with uninfected cells (panels a through c, respectively). These effects were partially restored by the addition of Ad.RSV.SERCA2a (multiplicity of infection of 10 pfu/cell) (FIG. 5). Similarly, the time course of shortening was significantly prolonged in cardiomyocytes infected with Ad.RSV.PL at a multiplicity of infection of 10 pfu/cell (time to 80% relaxation, from 387±22 to 780±44 milliseconds; P<0.5; n=12), whereas coinfection with Ad.RSV.SERCA2a restored the time course to normal (405±25 milliseconds, n=10, P>0.1 compared with uninfected cells).

Adenoviral gene transfer of phospholamban provides an attractive system for further elucidation of the effects of inhibiting SR Ca²⁺-ATPase on intracellular Ca²⁺ handling. A decrease in SR Ca²⁺ uptake rates is expected to lead to a smaller amount of Ca²⁺ sequestered by the SR, resulting in a smaller amount of Ca²⁺ release. In neonatal cardiomyocytes, a significantly prolonged Ca²⁺ transient and a higher resting [Ca²⁺] was observed reflecting the decreased Ca²⁺ uptake and a decrease in peak [Ca²⁺] levels reflecting less Ca²⁺ available for release. These results show that the SR Ca²⁺-ATPase is important during relaxation by controlling the rate and amount of Ca²⁺ sequestered and during contraction by releasing the Ca²⁺ that is taken up by the SR. Overexpression of both phospholamban and SERCA2a partially restored the Ca²⁺ transient; however, the time course of the Ca²⁺ transient was still prolonged in cardiomyocytes infected with both Ad.RSV.SERCA2a and Ad.RSV.PL. This finding was somewhat surprising, since the SR Ca²⁺-ATPase activity was restored to normal and even enhanced in cardiomyocytes infected with both Ad.RSV.SERCA2a and Ad.RSV.PL.

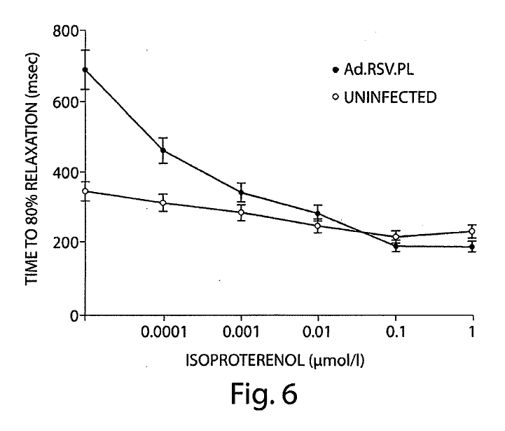

Phospholamban has been shown to play a key role in modulating the response of agents that increase cAMP levels in cardiomyocytes. Since phosphorylation of phospholamban reduces the inhibition to the SR Ca²⁺ pump, thereby enhancing the SR Ca²⁺-ATPase, we were specifically interested in evaluating the effects of β-agonism on the relaxation phase of the Ca²⁺ transient. In the basal state, the overexpression of phospholamban significantly prolongs the Ca²⁺ transient. As shown in FIG. 6, at maximal isoproterenol stimulation, the time course of the Ca²⁺ transients in the uninfected cardiomyocytes and the cardiomyocytes infected with Ad.RSV.PL were decreased to the same level. These findings show that phospholamban plays a major role in the enhanced relaxation of the heart to β-agonism. In addition, it corroborates these findings that phospholamban decreases the affinity of the SR Ca²⁺ pump for Ca²⁺ but does not decrease the maximal Ca²⁺ uptake rate.

### 5. Preparation of SR Membranes From Isolated Rat Cardiomyocytes

To isolate SR membrane from cultured cardiomyocyes, a procedure modified from Harigaya et al., Circ. Res., 1969, 25:781-794, as well as, Wienzek et al., 1992, 23:1149-1163, the contents of which are incorporated herein by reference, was used. Briefly, isolated neonatal cardiomyocytes were suspended in a buffer containing (mmol/L) sucrose 500, phenylmethylsulfonyl fluoride 1 and PIPES 20, at pH 7.4. The cardiomyocytes were then disrupted with a homogenizer. The homogenates were centrifuged at 500 g for 20 minutes. The resultant supernatant was centrifuged at 25,000 g for 60 minutes to pellet the SR-enriched membrane. The pellet was re-suspended in a buffer containing (mmol/L) KCl 600, sucrose 30, and PIPES 20, frozen in liquid nitrogen, and stored at −70° C. Protein concentration was determined in these preparations by a modified Bradford procedure, described in Bradford et al., Anal. Biochem., 1976, 72:248-260, the contents of which are incorporated herein by reference, using bovine scrum albumin for the standard curve (Bio-Rad).

### 6. Western Blot Analysis of Phospholamban and SERCA2a in SR Preparations

SDS-PAGE was performed on the isolated membranes from cell cultures under reducing conditions on a 7.5% separation gel with a 4% stacking gel in a Miniprotean II cell (Bio-Rad). Proteins were then transferred to a Hybond-ECL nitrocellulose for 2 hours. The blots were blocked in 5% nonfat milk in Tris-buffered saline for 3 hours at room temperature. For immunoreaction, the blot was incubated with 1:2500 diluted monoclonal anti-SERCA2 antibody (Affinity BioReagents) or 1:2500 diluted anti-cardiac phospholamban monoclonal IgG (UBI) for 90 minutes at room temperature. After washing, the blots were incubated in a solution containing peroxidase-labeled goat anti-mouse IgG (dilution, 1:1000) for 90 minutes at room temperature. The blot was then incubated in a chemiluminescence system and exposed to an X-OMAT x-ray film (Fuji Films) for 1 minute. The densities of the bands were evaluated using NIH Image. Normalization was performed by dividing densitometric units of each membrane preparation by the protein amounts in each of these preparations. Serial dilution of the membrane preparations revealed a linear relationship between amounts of protein and the densities of the SERCA2a immunoreactive hands (data not shown).

### 7. SR Ca²⁺-ATPase Activity

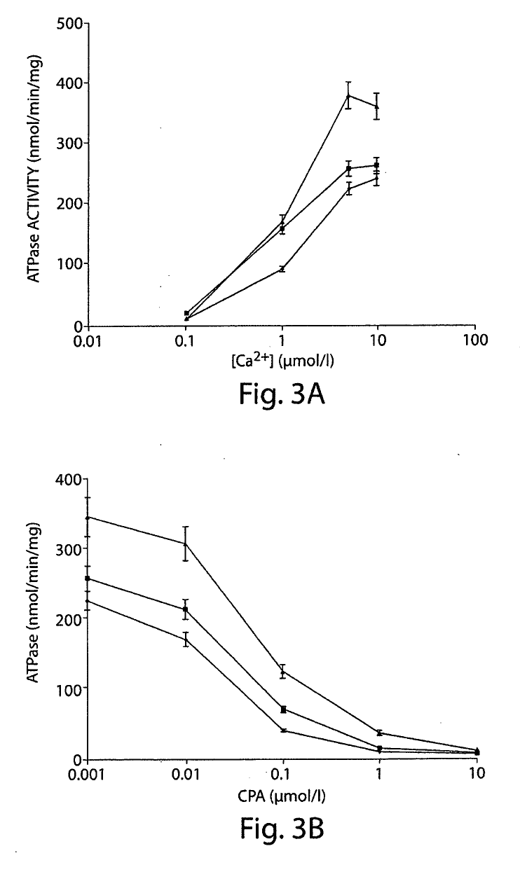

SR Ca²⁺-ATPase activity assays were carried out according to Chu A. et al., Methods Enzymol., 1988, 157:36-46, the contents of which are incorporated herein by reference, on the basis of pyruvate/NADH-coupled reactions. By use of a photomotor (Beckman DU 640) adjusted at a wavelength of 540 nm, oxidation of NADH (which is coupled to the SR Ca²⁺-ATPase) was assessed at 37° C. in the membrane preparations by the difference of the total absorbance and basal absorbance. The reaction was carried out in a volume of 1 mL. All experiments were carried out in triplicate. The activity of the Ca²⁺-ATPase was calculated as follows: Δabsorbance/6.22×−protein×time (in nmol ATP/mg protein×min). The measurements were repeated at different [Ca²⁺] levels. The effect of the specific Ca²⁺-ATPase inhibitor CPA at a concentration range of 0.001 to 10 μmol/L was also studied in those preparations, as described in Schwinger et al., Circulation, 1995, 92:3220-3228 and Baudet et al., Circ. Res., 1993, 73:813-819, the contents of which are incorporated herein by reference. As shown in FIG. 3A, the relationship between ATPase activity and Ca²⁺ was shifted to the right in the preparations from cardiomyocytes overexpressing phospholamban compared with the uninfected preparations without changing maximal Ca²⁺-ATPase activity. Coinfection with Ad.RSV.SERCA2a restored the Ca²⁺-ATPase activity and also increased the maximal Ca²⁺-ATPase activity. To verify that the ATPase activity measured from the membrane preparations was SR-related, the specific inhibitor CPA was used after maximally activating the SR Ca²⁺-ATPase with 10 μmol/L of Ca²⁺. As shown in FIG. 3B, CPA inhibited the SR Ca²⁺-ATPase activity in a dose-dependent fashion in all three membrane preparations (uninfected, Ad.RSV.PL, and d.RSV.PL+Ad.RSV.SERCA2a).

The SR Ca²⁺-ATPase plays a key role in excitation-contraction coupling, lowering Ca²⁺ during relaxation in cardiomyocytes, and “loading” the SR with Ca²⁺ for the subsequent release and contractile activation. The Ca²⁺-pumping activity of this enzyme is influenced by phospholamban. In the unphosphorylated state, phospholamban inhibits the Ca²⁺-ATPase, whereas phosphorylation of phospholamban by cAMP-dependent protein kinase and by Ca²⁺ calmodulin-dependent protein kinase reverses this inhibition. Therefore, an increase in phospholamban content should decrease the affinity of the SR Ca²⁺ pump for Ca²⁺. As shown in FIG. 4, overexpression of phospholamban shifted the relationship between SR Ca²⁺-ATPase activity and Ca²⁺ to the right, indicating a decrease of the sensitivity of the SR Ca²⁺ to pump to Ca²⁺. However, there was no change in the maximal Ca²⁺-ATPase activity in the Ad.RSV.PL-infected cardiomyocytes. This shows that the Vₘₐₓ of the Ca²⁺-ATPase of cardiac SR is not altered by interaction with phospholamban and phosphorylation, and that in mice overexpressing phospholamban, the affinity of the SR Ca²⁺ pump for Ca²⁺ was decreased but that the maximal velocity of the SR Ca²⁺ uptake was not changed. From the present experiment, it can also be concluded that phospholamban affects the affinity of the SR Ca²⁺ pump for Ca²⁺ without changing the maximal ATPase activity. The concomitant overexpression of SERCA2a and phospholamban restored the ATPase activity and also increased the maximal Ca²⁺-ATPase activity. This brings further evidence that the expression of additional SR Ca²⁺-ATPase pumps can overcome the inhibitory effects of phospholamban.

### 8. Statistical Analyses

Data were represented as mean±SEM for continuous variables. Student's test was used to compare the means of normally distributed continuous variables. Parametric one-way ANOVA techniques were used to compare normally distributed contiguous variables among uninfected groups of cells, Ad.RSV.βgal-infected cells, Ad.RSV.PL-infected cells, and Ad.RSV.SERCA2a-infected cells.

### 9. Adenoviral Somatic Gene Transfer

Rats and mice were anesthetized with intraperitoneal pentobarbital and placed on a ventilator. The chest was entered form the left side through the third intercostal space. The pericardium was opened and a 7-0 suture placed at the apex of the left ventricle. The aorta and pulmonary artery were identified. A 22 G catheter containing 200 μl of adenovirus was advanced from the apex of the left ventricle to the aortic root. The aorta and pulmonary artery were clamped distal to the site of the catheter and the adenovirus solution was injected as shown in FIG. 9. The clamp was maintained for 10 seconds while the heart was pumping against a closed system (isovolumically). This allowed the adenovirus solution to circulate down the coronary arteries and perfuse the whole heart without direct manipulation of the coronaries. After the 10 seconds, the clamp on the aorta and the pulmonary artery was released, the chest was evacuated from air and blood and closed. Finally, the animals were taken off the ventilator.

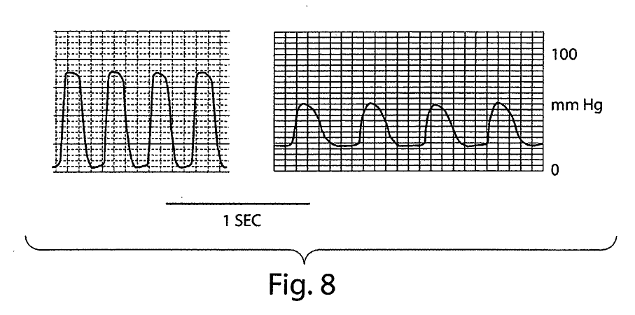

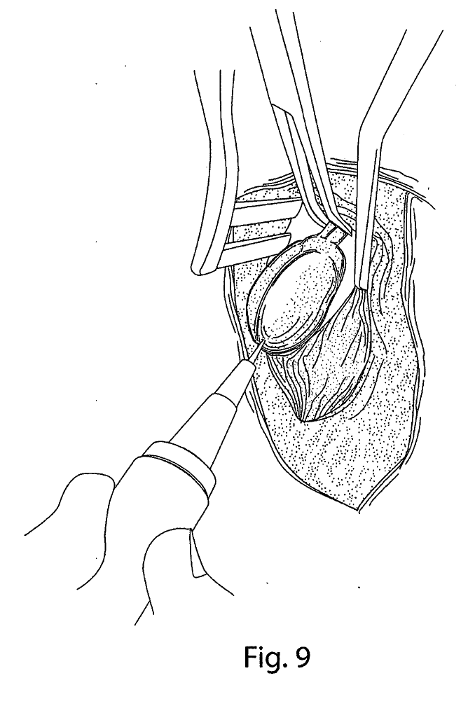

The expression pattern seen after direct injection is localized, whereas the catheter-based technique is essentially homogeneous. The pressure tracing of the Ad.PL transduced hearts displayed a markedly prolonged relaxation and reduced pressure development as shown in FIG. 8.

### 10. Gene Transfer of the Sarcoplasmic Reticulum Calcium ATPase Improves Left Ventricular Function in Aortic-Banded Rats in Transition to Failure

In human and experimental models of heart failure, sarcoplasmic reticulum Ca²⁺ ATPase (SERCA2a) activity has been shown to be significantly decreased. In this example, the ability of SERCA2a expression to improve ventricular function in heart failure was investigated by creating an ascending aortic constriction in 10 rats. After 20-24 weeks, during the transition from left ventricular hypertrophy to failure, 200 μl of a solution containing 5×10⁹ plaque forming units of replication-deficient adenovirus carrying SERCA2a (Ad.SERCA) (n=4) or the reporter gene β-galctosidase (Ad.βgal) (n=6) were injected intracoronary via the catheter-based technique described supra. Two days after the procedure, the rats underwent open chest measurement of left ventricular pressure. Heart rate (HR), left ventricular end-diastolic pressure (LVEDP), and left ventricular systolic pressure (LVSP) were measured. Peak+dP/dt and −dP/dt were calculated. As shown in Table 2, the magnitudes of peak+dP/dt and −dP/dt which are indices of systolic and diastolic function were markedly increased in hearts transduced with the SERCA2a carrying adenovirus. Therefore, this example indicates that overexpression of SERCA2a in a rat model of pressure-overload hypertrophy in transition to failure improved left ventricular systolic and diastolic function.

TABLE 2
Final echocardiography and hemodynamic data.

| | | LVEDP | LVSP | +dP/dt | |
|---|---|---|---|---|---|
| | HR (bpm) | (mmHg) | (mmHg) | (mmHg/sec) | −dP/dt (mmHg/sec) |
| Ad. βgal | 416 ± 46 | 6 ± 4 | 114 ± 16 | 5687 ± 1019 | −5023 ± 1803 |
| Ad. SERCA | 450 ± 53 | 9 ± 3 | 148 ± 40 | 9631 ± 3568^{#} | −8385 ± 980^{#} |

^{#}p < compared to Ad. βgal
Values are mean ± S.D.
β-gal, pigs receiving β-galactosidase gene:
SERCA2a, pigs receiving SERCA2a gene;
Coil, permanent coronary artery occlusion;
I/R, ischemia-reperfusion;
Sham, no coronary artery occlusion.
AW, anterior wall thickness in diastole (d) and systole (s);
LVEDDLV end-diastolic diameter;
LVESD, LV end-systolic diameter;
SF, shortening fraction;
HR, heart rate;
SAP, systolic aorticpressure;
CO, cardiac output;
LVEDP, LV end-diastolic pressure;
dP/dt, first derivative of LV pressure.
*p < 0.05 vs. β-gal-Coil + 0.05 vs. SERCA2a-Coil;
‡p < 0.05 vs. β-gal I/R.

### 11. Gene Transfer of Antisense of Phospholamban Improves Contractility in Isolated Cardiomyocytes in Rat and Human

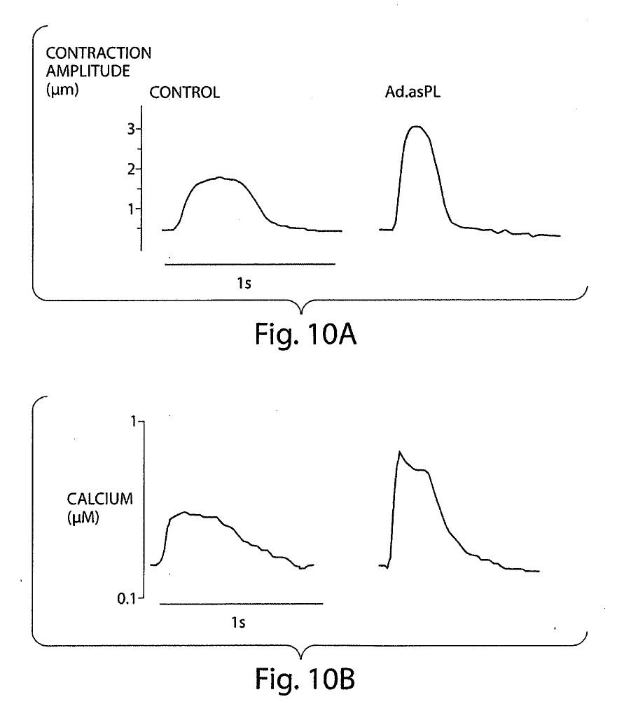

A. Delayed cardiac relaxation in failing hearts is attributed to a reduced activity of the Sarcoplasmic Reticulum Calcium ATPase. Phospholamban inhibits SERCA2a activity and is, therefore, a potential target to improve cardiac function. In this Example, an adenovirus carrying the full length antisense cDNA of phospholamban (Ad.asPL) was constructed using the methods described above. This construct was then used to infect neonatal cardiac myocytes as described in Example 3. As indicated in FIG. 10A, infection of neonatal cardiac myocytes with the Ad.asPL construct increased the contraction amplitude and significantly shortened the time course of the contraction. The adenovirus-mediated gene transfer of the antisense cDNA for phospholamban also resulted in a modification of intracellular calcium handling and shortening in myocardial cells (see FIG. 10B) indicating that such vectors can be used for increasing the contractility of myocardial cells in heart failure.

B. Since human heart failure is mainly due to coronary artery disease or is idiopathic in nature, we ablated phosholamban by antisense strategies using adenoviral gene transfer in isolated ventricular cardiac myocytes from eight patients with end-stage heart failure of various etiologies (idiopathic, ischemic and hypertrophic). The co-expression of green fluorescent protein GFP allowed us to identify the cells that were infected and expressing the transgene after 48 hours.

Following isolation, failing human cardiomyocytes were infected with an adenovirus carrying antisense phospholamban. Forty-eight hours after infection, a cardiomyocyte is visualized with white light and at 510 nm with single excitation peak at 490 nm of blue light. Co-expression of GFP demonstrated visually the ablation of phospholamban in the cell. Recordings were performed from cardiomyocytes isolated from a donor nonfailing heart and from a failing heart infected with either an adenovirus expressing green fluorescent protein, Ad.GFP or carrying the antisense of phospholamban, Ad.asPL, stimulated at 1 Hz at 37° C. The failing cell had a characteristic decrease in contraction and prolonged relaxation along with a prolonged Ca²⁺ transient. Ablation of phospholamban in the failing cardiomyocyte normalized these parameters. Ablation of phospholamban in failing cardiomyocytes induced a faster contraction velocity (15.4±2.7 vs. 6.9±2% shortening/sec, p=0.008) and enhanced relaxation velocity (18.6±4.4 vs. 6.6±3.7, p=0.01).

These results show that regardless of etiology, in human heart failure, improving calcium cycling by decreasing phospholamban inhibition to SERCA2a, restores contractility in failing ventricular cells of different etiologies. These findings also extend previous results that overexpression of SERCA2a improves contractile function in human failing cardiac myocytes. Finally, these findings underscore the importance of validating experimental results from murine models in relevant human tissues.

### 12. Gene Transfer of the Sarcoplasmic Reticulum Calcium ATPase Improves Survival in Aortic-Banded Rats in Transition to Failure

Pharmacological agents that increase contractility have been repeatedly shown to worsen survival in patients with congestive heart failure and to increase the energetic requirements on the heart (O'Connor et el. (1999). Am Heart J 138 (1 Pt 1):78-86). Since the heart performs uninterrupted biochemical and mechanical work, it requires a continuous supply of energy in the form of ATP by mostly oxidative metabolism under normal conditions with major energy reserve molecule represented by phosphocreatine (PCr). In the normal heart, although the majority (60%) of the energy consumption is due to cross-bridge cycling, relaxation requires an energy expenditure of 15% to remove Ca²⁺ from the cytoplasm. This high level of free energy |ΔGp| required by the SERCA2a reaction is directly related to the magnitude of the Ca²⁺ gradient across the SR (Tian et al. (1998) Am J Physiol 275 (6 Pt 2):H2064-71). Failing hearts have a reduced ratio PCr/ATP in human as well as in animal models of heart failure so that less energy reserve is available for the cellular processes. This decrease in energy reserve has been shown to be by itself a predictor of mortality in patients with dilated cardiomyopathy (Neubauer et al. (1997) Circulation 96 (7):2190-6).

In this Example, unlike other pharmacologic agents that increase inotropy, reconstitution of normal levels of SERCA2a by adenoviral gene transfer improves contractile performance as well as survival in aortic banded rats with developed heart failure without adversely affecting energetics possibly by reducing the intracellular diastolic Ca²⁺ overload.

### Experimental Protocols for Examples 1-13

A. Construction of Recombinant Adenoviruses

We constructed an adenovirus containing SERCA2a and GFP controlled by separate CMV promoters (Ad.SERCA2a). An adenovirus containing both β-galactosidase and GFP controlled by separate CMV promoters (Ad.βgal-GFP) was used as control as described earlier (Haq et al. (2000) J Cell Biol 151 (1):117-130). The titer of stocks used for these studies measured by plaque assays were: 3×10¹¹ pfu/ml for Ad.βgal-GFP and 1.8×10¹¹ pfu/ml for Ad.SERCA2a with a particle/pfu ratio of 8:1 and 18:1 respectively (viral particles/ml determined using the relationship one absorbance unit at 260 nm is equal to 10¹² viral particles/ml). These recombinant adenoviruses were tested for the absence of wild-type virus by PCR of the early transcriptional unit E1.

B. Aortic Banding

Four-week old Sprague Dawley rats (70-80 g) were obtained from Taconic Farms. After 2-3 days of acclimatization, the rats were anesthetized with intraperitoneal pentobarbital (65 mg/kg) and placed on a ventilator. A suprasternal incision was made exposing the aortic root and a tantalum clip with an internal diameter of 0.58 mm (Weck, Inc.) was placed on the ascending aorta. Animals in the sham group underwent a similar procedure without insertion of a clip. The supraclavicular incision was then closed and the rats were transferred back to their cages. The supraclavicular approach was performed because during gene delivery a thoracotomy is necessary and by not opening the thorax during the initial aortic banding avoids adhesions when gene delivery is performed thereby decreasing the morbidity of the procedure.

Animals were initially divided into two groups: one group of 45 animals with aortic banding and a second group of 42 animals which were sham-operated. Three animals did not survive the initial operation in the aortic banding group and 2 animals did not survive in the sham-operated group. In the animals which were aortic banded we waited 26-28 weeks for the animals to develop left ventricular dilatation prior to cardiac gene transfer. In this last group as well as in the sham-operated group, fourteen animals did not undergo gene transfer and were followed longitudinally. The rest of the animals underwent adenoviral gene transfer with either Ad.SERCA2a or Ad.bgal-GFP.

C. ³¹P NMR Measurements

NMR Spectroscopy

Stable energetic state in rat hearts was confirmed from 31p NMR signals of phosphocreatine, ATP, and inorganic phosphate as described in Lewandowski et al. ((1995) American J Physiol 269 (1 Pt 2):H160-8). NMR data was collected on a Bruker 400 MHz spectrometer interfaced to a 9.4 tesla, vertical bore, superconducting magnet. ³¹P spectra were obtained from isolated hearts perfused within a broad-band, 20 mm NMR probe (Bruker Instruments). ³¹P-NMR spectra were acquired in 128 scans using a 161 MHz, 45° C. excitation pulse, a 1.8 s repetition time, 35 ppm sweep width, and 8 K data set. Post processing of the summed free induction decay (FID's) NMR data included 20 Hz line broadening, Fourier transformation, and phase correction. Peak assignments were referenced to the well established resonance signal of PCr at 0 ppm, with identification and assignment of the α, β, and .gamma. phosphate signals of ATP. Signal intensity was determined using NMR-dedicated data analysis.

Isolated, Perfused Rat Heart Preparation:

Hearts were retrograde perfused from a 100 cm hydrostatic perfusion column with modified Krebs-Henseleit buffer (116 mM NaCl, 4 mM KCl, 1.5 mM CaCl₂, 1.2 mM MgSO₄, 1.2 mM NaH₂PO₄, and 25 mM NaHCO₃, equilibrated with 95% O₂/5% CO₂ at 37° C.) that contained 5 mM glucose in a 2 liter reservoir. A polyethylene catheter was inserted into the pulmonary artery allowing collection of coronary effluent for measurement of oxygen consumption with a blood-gas analysis machine. Hearts spontaneously beat, contracting against a fluid-filled intraventricular balloon connected to a pressure transducer and inflated to an end diastolic pressure of 5 mm Hg. The isolated hearts were placed in a borosilicate glass vial. A 10-15 ml volume of coronary effluent bathed the heart. Temperature was maintained at 37° C. with both perfusate temperature and a thermal control unit interfaced to the NMR system.

D. Serial Echocardiographic Assessment

After eighteen weeks of banding, serial echocardiograms were performed on a weekly basis. Animals were anesthetized with pentobarbital 40 mg/kg intra-peritoneally, and the anterior chest shaved. Transthoracic M-mode and two-dimensional echocardiography was performed with a Hewlett-Packard Sonos 5500 imaging system (Andover, Mass.) with a 12 MHz broadband transducer. A mid-papillary level left ventricular short axis view was used and the images were stored digitally. Measurements of posterior wall thickness, left ventricular diastolic dimension and fractional shortening were performed off-line. The epicardial surface of the anterior wall was not reliably visualized in all animals. Gene transfer was performed in all animals within 3 days of detection of a drop in fractional shortening of >25% compared to the fractional shortening at 18 weeks post-banding. In the sham operated rats, gene delivery was performed at 27 weeks.

E. Adenoviral Delivery Protocol

The group of animals subjected to aortic banding were further subdivided in three additional groups of sixteen, twelve, and fourteen receiving respectively Ad.SERCA2a, Ad.bgal-GFP, or no adenovirus. The group of sham-operated animals was also subdivided into three groups of fourteen, twelve, and fourteen Ad.SERCA2a, Ad.bgal-GFP, or no adenovirus. The adenoviral delivery system has been described in Miyamoto et al. ((2000) Proc Natl Acad Sci USA 97 (2):793-8). Briefly, after anesthetizing the rats and performing a thoracotomy, a 22 G catheter containing 200 ml of adenoviral solution (10¹⁰ pfu) was advanced from the apex of the left ventricle to the aortic root. The aorta and main pulmonary artery were clamped for 20 seconds distal to the site of the catheter and the solution injected, then the chest was closed, the animals were extubated and transferred back to their cages.

F. Measurements of Left Ventricular Volume & Elastance

Prior to euthanasia, rats in the different treatment groups were anesthetized with 65 mg/kg of pentobarbital and mechanically ventilated. After thoracotomy, a small incision was then made in the apex of the left ventricle and a 1.4 French high fidelity pressure transducer (Millar Instruments, Tex.) introduced into the left ventricle. Pressure measurements were digitized at 1.0 kHz and stored for further analysis using commercially available software (Sonolab, Sonometrics Co., Alberta, Canada) and four 0.7 mm piezoelectric crystals (Sonometrics Co., Canada) were placed over the surface of the left ventricle along the short axis of the ventricle at the level of the mitral valve and at the apex of the left ventricle to measure the inter-crystal distances. The left ventricular volume was derived using a mathematical model using CARDIOSOFT (Sonometrics Co., Canada). Left ventricular pressure-volume loops were generated under different loading conditions by clamping the inferior vena cava. The end-systolic pressure-volume relationship was obtained by producing a series of pressure dimension loops over a range of loading conditions and connecting the upper left hand corners of the individual pressure-dimension loops to generate the maximal slope.

G. Western Blot Analysis

SDS-PAGE was performed on the tissue lysate under reducing conditions on 7.5% separation gels with a 4% stacking gel in a Miniprotean II cell (BIORAD). Proteins were then transferred to a Hybond-ECL nitrocellulose for 2 hours and blocked in 5% nonfat milk for 3 hours. For immunoreaction, the blots were incubated with 1:2,500 diluted monoclonal antibodies to either SERCA2a (MA3-919; Affinity BioReagents, CO), or 1:1,000 diluted anti-calsequestrin (MA3-913; Affinity BioReagents) for 90 minutes at room temperature. After washing, the blots were exposed for 1 hour to HRP conjugated anti mouse antibody for chemo-luminescent detection.

H. SR Ca²⁺ ATPase Activity

SR Ca²⁺ ATPase activity assays were carried out based on a Pyruvate/NADH coupled reactions as previously described (Miyamoto, supra). Using a photometer (Beckman DU 640) adjusted at a wavelength of 340 nm, oxidation of NADH (which is coupled to the SR Ca²⁺-ATPase) was assessed at 37° C. in triplicates at different [Ca²⁺]. The reaction was carried out in a volume of 1 ml. Ca²⁺-ATPase activity was calculated as: ΔAbsorbence/(6.22×protein×−time) in nmol ATP/(mg protein×min).

I Statistics

All values are presented as mean±sd. A two-factor ANOVA was performed to compare the different hemodynamic parameters among the different groups. For the echocardiography data, where the variables were examined at various intervals, ANOVA with repeated measures was performed. Comparison of survival in the different groups of animals was analyzed by a log-rank test with the Kaplan-Meier method. Statistical significance was accepted at the level of p<0.05.

Effect on Survival

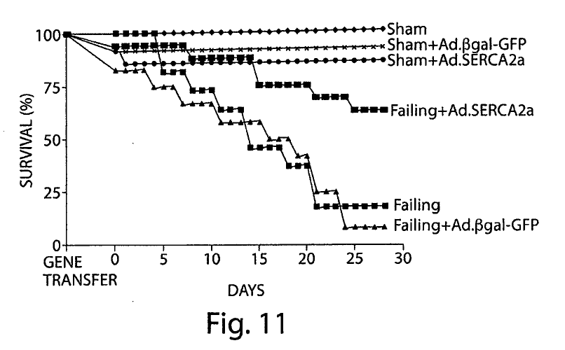

FIG. 11 shows the survival curve for the six different groups studied. The sham operated animals did not show any premature mortality. The sham operated animals that were either infected with Ad.bgal-GFP or Ad.SERCA2a had early mortalities related to the surgical intervention of cardiac gene transfer, but then the survival curves leveled off for both sham+Ad.bgal-GFP and sham+Ad.SERCA2a. In the failing group, the non-infected animals had a survival curve that decreased steadily and at 4 weeks the survival rate was only 18% (p<0.0005 compared to sham). In the failing group+Ad.bgal-GFP the survival curve also decreased and at 4 weeks the survival rate was only 9% (p<0.001 compared to sham+Ad.bgal-GFP). However, in the failing group+Ad.SERCA2a, the survival curve was significantly improved compared to failing+Ad.SERCA2a (p<0.001 compared to failing+Ad.bgal-GFP).

Characterization of Animals

Following 18 weeks of aortic banding, the animals showed echocardiographic signs of left ventricular hypertrophy including an increase in wall thickness (both posterior and septal), an increase in posterior wall thickness, a decrease in left ventricular dimensions and an increase in fractional shortening as shown in Table 3. Of note at that time the animals showed no clinical signs of heart failure. After 26-27 weeks of banding, these animals had uniformly 1) small pericardial effusions, 2) pleural effusions, 3) an increase in lung weight, 4) ascites, and 5) dyspnea at rest all indicative signs of developed heart failure. Echocardiographically, LV end-diastolic dimensions increased and fractional shortening decreased.

TABLE 3
Echocardiographic Measures in Rats after Sham Surgery or Aortic Banding

| | Septum | PW | LVEDD | LVESD | FS |
|---|---|---|---|---|---|
| | (mm) | (mm) | (mm) | (mm) | (%) |
| Sham | 14.9 ± 1.1 | 13.5 ± 1.0 | 66.8 ± 3.8 | 40.4 ± 6.0 | 40.0 ± 6.3 |
| Aortic banding | 20.1 ± 3.9\hat{ } | 19.8 ± 2.8\hat{ } | 61.9 ± 6.4*\hat{ } | 34.0 ± 6.2*\hat{ } | 46.0 ± 8.2*\hat{ }# |
| (18 weeks) | | | | | |
| Aortic banding | 19.7 ± 2.8‡ | 18.5 ± 2.3‡ | 69.5 ± 6.3# | 45.1 ± 6.9\hat{ } | 36.0 ± 10.4# |
| (27 weeks) | | | | | |

PW: posterior wall thickness during diastole
LVDD: Left ventricular Diameter during diastole
LVSD: Left ventricular Systolic Diameter during Systole
FS: Fractional shortening
*p < 0.0005 vs Aortic banding (27 weeks)
‡p < 0.005,
\hat{ }p < 0.005,
^{#}p < 0.05 vs control

Cardiac Gene Transfer & SERCA2a Expression

We first examined the expression of SERCA2a 28 days following adenoviral gene transfer. There was a decrease in SERCA2a in failing rats compared to sham operated rats. The protein expression of SERCA2a was decreased in failing rat left ventricles when compared to SERCA2a levels of sham left ventricles. Adenoviral gene transfer of SERCA2a in failing hearts increased SERCA2a protein expression restoring it to levels observed in the nonfailing hearts. The protein levels were normalized to calsequestrin which did not change among the different groups. To evaluate whether other tissues are infected we histologically examined sections of aorta, liver, and lung following infection with the cardiac specific Ad.SERCA2a. There was no evidence of SERCA2a expression in the aorta, in the liver and lungs. In the infected rat hearts there was no evidence of disruption of normal myocardial architecture or collagen deposition.

Thus, we restored SERCA2a protein to normal levels in failing hearts. In addition, we showed that the expression of SERCA2a to normal levels was sustained for up to four weeks. This seemed somewhat surprising since first generation adenoviruses induce transient expression peaking at 7-10 days and disappearing after 10 days 23. However, endogenous turnover of SERCA2a is about 14-15 days in young rats and longer in older rats 24 which would explain the sustained levels of SERCA2a.

SR Ca² ATPase Activity

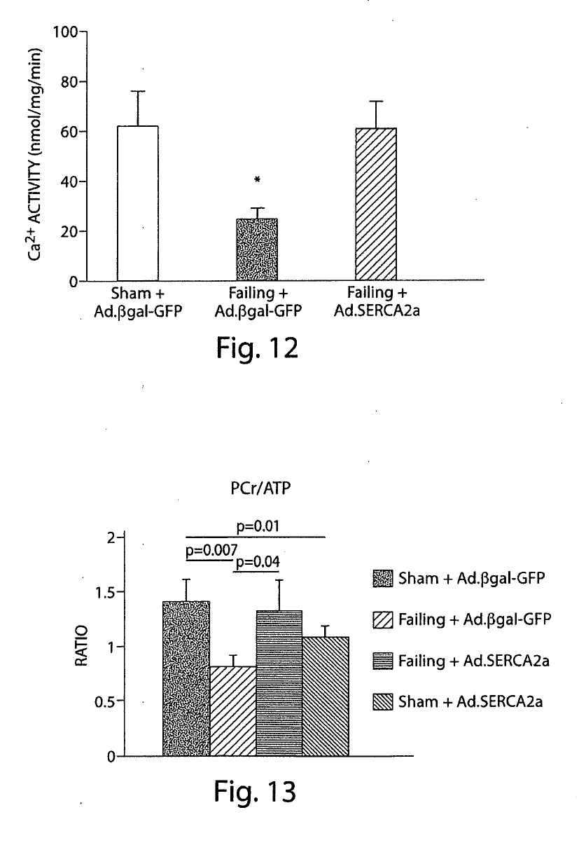

We measured SR ATPase activity at a calcium concentration of 10 mM in 1) sham+Ad.bgal-GFP 2) failing+Ad.bgal-GFP, and 3) failing+Ad.SERCA2a. As shown in FIG. 12, there was a decrease in maximal ATPase activity in the failing group. Gene transfer of SERCA2a restored ATPase activity back to normal levels in the failing group four weeks following gene transfer.

SERCA2a Expression and Cardiac Energetics

Representative ³¹P—NNR spectra obtained from three groups of rats: 1) sham+Ad.bgal-GFP, 2) failing+Ad.bgal-GFP, 3) failing+Ad.SERCA2a are shown in FIG. 13. These spectra show that the ratios of total amounts PCr to ATP are lower in the failing heart when compared with the sham heart. The integrated area for Pi was also increased in the failing heart. The overexpression of SERCA2a in failing heart restored and normalized both the content of PCr and ATP while the integrated area for Pi was reduced. Interestingly we found that overexpression of SERCA2a in sham operated animals induces a reduction in PCr:ATP ratio (FIG. 13).

Thus, restoring SERCA2a levels to normal induced an improvement in the creatine phosphate to ATP ratio. The findings of improved cardiac energetics in developed heart failure was somewhat surprising since overexpression of SERCA2a would be anticipated to increase ATP hydrolysis thereby driving creatine phosphate down. Indeed, this increase in ATP hydrolysis is consistent with our observation of reduced PCr/ATP in the group of sham-operated hearts that were overexpressing SERCA2a. These results are also consistent with previous results showing that PCr/ATP was decreased in the phospholamban-deficient hearts relative to the wild-type hearts (Chu et al. (1996) Circ Res 79 (6):1064-76). In heart failure, however, elevated calcium levels would increase energy demand. Furthermore, the thermodynamic reserve for the SR Ca²⁺-ATPase reaction is limited and in order to maintain the normal Ca²⁺ gradient, the SR Ca²⁺-ATPase reaction requires a |ΔGp| of at least 52 kJ/mol, 85-90% of it from ATP. Therefore, of all the ATPase reactions in cardiac myocytes, the SR Ca²⁺-ATPase reaction is the most vulnerable to a decrease in |ΔGp|.

Effects of SERCA2a Overexpression on LV Volumes and Elastance

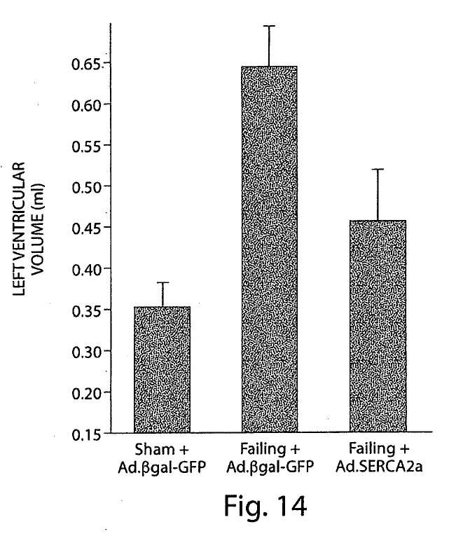

To determine left ventricular function, pressure-ventricular analysis was performed in a subset of animals. LV volumes were significantly increased in the failing rats (0.64±0.05 vs. 0.35±0.03 ml, p<0.02). Overexpression of SERCA2a normalized LV dimensions (0.46±0.07 ml) in the failing hearts (FIG. 14). To alter loading conditions, we clamped the inferior vena cava in the open-chested animals thereby reducing ventricular volume. This enabled us to calculate the end-systolic pressure volume relationship using a series of measurements made under varying pre-load conditions. The slope of the end-systolic pressure dimension relationship was lower in failing hearts infected with Ad.bgal-GFP compared to control indicating a diminished state of intrinsic myocardial contractility: 450±71 mmHg/ml vs. 718±83 mmHg/mm (p<0.02). Overexpression of SERCA2a restored the slope of the end-systolic pressure dimension relationship to control levels (691±91 mmHg/ml, p<0.03 compared to failing+Ad.bgal-GFP; p>0.1 compared to sham+Ad.bgal-GFP).

Effect on Morphological Parameters

As shown in table 4, the failing hearts had a significant increase in heart mass when normalized to either tibial length or to body mass. Tibial length which was used as an index of growth independent of body weight was uniformly constant across the different groups. Body mass was also not significantly different across the different groups. Overexpression of SERCA2a in the failing heart did not have a significant effect on left ventricular mass whether normalized to tibial length or body mass.

TABLE 4
Morphometric Analyses

| | Sham + | | Failing + | |
|---|---|---|---|---|
| | Ad.βgal- | Sham + | Ad.βgal- | Failing + |
| | GFP | Ad.SERCA2a | GFP | Ad.SERCA2a |
| HW/BW × | 3.7 ± 0.3 | 4.4 ± 0.6 | 4.4* ± 0.5 | 4.3* ± 0.4 |
| 10⁴ | | | | |
| HW/TL × | 44.8 ± 4.3 | 55.3 ± 6.2 | 50.8* ± 4.4 | 50.3* ± 6.3 |
| 10² (g/mm) | | | | |

HW: heart weight
BW: Body weight
TL: Tibial length
*p < 0.05 compared to Sham + Ad.GFP

Survival Following Gene Transfer

Herein, we show that restoration of SERCA2a expression by cardiac gene transfer in vivo improves not only contractile function but also survival and cardiac energetics. In addition, cardiac gene transfer of SERCA2a induced a reversal of adverse remodeling in the failing hearts.

In this model of heart failure SERCA2a overexpression improved parameters of inotropy and normalized contractile reserve. These effects translate into an inotropic intervention. However, other inotropic interventions have been shown clinically to increase mortality in chronic heart failure in numerous trials (Stevenson (1998) New England Journal of Medicine 339 (25):1848-50). There are, however, significant differences between increasing inotropy with pharmacological agents that usually increase cAMP and enhancing inotropy with the overexpression of SERCA2a. Unlike agents that increase cAMP, thereby increasing intracellular Ca²⁺, reconstituting normal SERCA2a levels decreases diastolic intracellular Ca²⁺ by increasing uptake into the SR and enhancing Ca²⁺ release. Beyond the contractile benefits of lowering diastolic Ca²⁺, it has been shown that sustained elevations of resting Ca²⁺ lead to activation of serine-threonine phosphatases including calcineurin inducing hypertrophy and cell death in cells (Lim (1999) Nature Medicine 5 (3):246-7). Therefore a decrease in diastolic Ca²⁺ may in effect decrease the stimulation of phosphatases and reduce the pro-apoptotic and pro-hypertrophy signaling. Heart failure is associated with an increased incidence of ventricular arrhythmias and triggered activity is a probable mechanism of arrhythmogenesis in heart failure. The increase in intracellular calcium secondary to SERCA2a downregulation increases the arrhythmogenic potential. Preventing an increase in intracellular calcium by overexpression of SERCA2a prevents the induction of triggered activity. Furthermore, improvement in energetics is another important finding in these examples which may have a direct influence on survival.

Our results demonstrate that restoring SERCA2a expression can improve not only systolic and diastolic performance in failing hearts but also survival and cardiac energetics. Furthermore, SERCA 2a normalization halts the adverse remodeling that occurs with congestive heart failure.

### Example 13

### Specificity of AAV6 to Heart Tissue

We tested the ability of different serotypes of AAV to deliver an exogenous gene to the heart. Using the cross-clamping technique described below, we injected rat hearts with 10¹² genomes of different AAV subtypes (1-6) carrying beta galactosidase under the CMV promoter. Rats were anesthetized with intraperitoneal pentobarbital and placed on a ventilator. The chest was entered from the left side through the third intercostal space. The pericardium was opened and a 7-0 suture placed at the apex of the left ventricle. The aorta and the pulmonary artery were identified. A 22 G catheter containing 200 μl of adenovirus was advanced from the apex of the left ventricle to the aortic root. The aorta and the pulmonary arteries were clamped distal to the site of the catheter and the solution injected. The clamp was maintained for 10 seconds, while the heart pumped against a closed system (isovolumically). This allows the solution that contains the adenovirus to circulate down the coronary arteries and perfuse the heart, without direct manipulation of the coronaries. After 40 seconds, the clamp on the aorta and the pulmonary artery was released. After removal of air and blood, the chest was closed, animals were extubated, and transferred back to their cages. Three to four rats were used at each time point.

We measured the expression of beta galactosidase in the ventricle (via X-gal activity) at various time intervals after AAV injections. We found that AAV6 has some surprising and unexpected properties relative to other AAVs. As shown in FIG. 15, AAV6 conferred the fastest gene expression, as well as the most specific and efficient expression in the heart. Other AAVs, however, may be useful for other applications, e.g. ones in which a different course of expression is desired.

### Example 14

### Gene Transfer in Pigs and Sheep

Percutaneous antegrade intracoronary gene transfer with concomitant coronary vein blockade (CVB) was performed in both sheep, and swine models. Using these large animal models we have developed a new technique of gene transfer. The left anterior descending artery (LAD) or the left circumflex artery (LCX) was cannulated and occluded with a standard angioplasty balloon. One-minute ischemic preconditioning in both the LAD and the LCX distribution (by blockade of the LAD and the LCX) was performed to allow increased viral dwell time in this model. Following the preconditioning protocol, the great coronary vein (GCV) or one of its branches was cannulated and temporarily occluded with a standard wedge balloon catheter. CVB was performed globally, implying occlusion of the proximal GCV and thus occluding venous drainage in both the LAD and LCX distribution, or selectively, in which case the anterior interventricular vein (AIV) was occluded during LAD delivery and similarly, the ostium of the middle cardiac vein (MCV) was occluded during LCX delivery. With both the arterial and the venous balloons inflated, percutaneous antegrade intracoronary gene transfer was performed by injection through the center lumen of the inflated angioplasty balloon with an adeno-associated virus carrying β-galactosidase (AAV6.β-gal) (n=5).

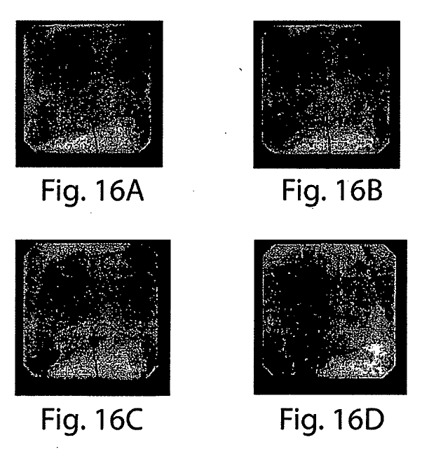

FIG. 16 shows the placement of catheters in this technique.

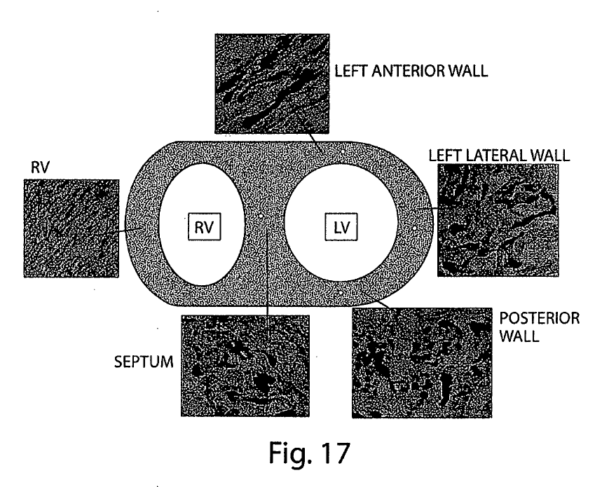

Twelve weeks following gene transfer with AAV6.CMV.βgal, myocardial sections of 10 μm were obtained from the septal, anterior, left lateral, posterior, and right ventricular walls. These sections were fixed with a phosphate-buffered solution (PBS), containing 0.5% glutaraldehyde for 30 minutes, and then in PBS with 30% sucrose for 30 minutes. The sections were then incubated overnight in a solution containing 5-bromo-4-chloro-3-indolyl α-D-galactopyranoside (X-gal). The results are shown in FIG. 17. FIG. 17 shows an extensive transfer of β galactosidase throughout the myocardium. FIG. 17, therefore, shows that the antegrade transduction of AAV6.CMV.β-gal at a concentration of 5×10¹⁴ genomes/ml with the global CVB resulted in a significant gene expression in the targeted myocardium, demonstrating feasibility and safety in a large animal model.

### Example 15

### Restoration of Normal Ventricular Function Following Gene Transfer of SERCA2a Using AAV6.CMV.SERCA2a

This study was carried out according to the Guidelines for the Care and Use of Laboratory Animals, approved by the Massachusetts General Hospital, Subcommittee on Research Animal Care. In a first set of experiments, 22 normal pigs underwent creation of mitral valve regurgitation (MVR). A carotid approach was used to insert a percutaneous biotome through an 8 Fr sheath. The biotome was advanced in a retrograde fashion through the aortic valve and through the left ventricular cavity towards the posterior wall. The cordae of the posterior papillary muscle were cut to create mitral valve regurgitation. The advancement and the positioning of the catheter and the cordae were performed under 2D echocardiographic monitoring. Color Doppler echocardiography was used to quantify the degree of MVR for inter-animal homogeneity of injury. Serial echocardiograms were performed in anesthetized animals. Transthoracic M-mode and 2D echocardiography was performed using a General Electric ultrasound system with a 3-MHz transducer. A mid-papillary level LV short-axis view was used, and measurements of posterior wall thickness, LV systolic and diastolic dimension, and fractional shortening were collected at baseline just before of MVR creation, one month after the MVR creation just before gene delivery and just before sacrifice.

Three months following the MVR creation, 19 pigs had survived and underwent gene transfer of either AAV6.CMV.SERCA2a or AAV6.CMV.bgal. A right femoral approach was used to advance a 50 cm 8 Fr modified AL1 (Cordis Corporation, Miami, Fla.) in the coronary sinus. An 110 cm 5 Fr wedge-balloon (Allow International Inc, Reading, Pa.) was advanced via the GCV to the AIV over a 0.025 inch guidewire (Terumo Corporation, Tokyo, Japan). Coronary venous pressure was monitored during the catheter manipulation. The wedge balloon was inflated until coronary venous occlusion was confirmed both by angiography and a rise in the coronary venous pressure. Coronary angiography was performed before gene delivery following 100 μg nitroglycerin injection. A 9 mm length, 3.5 mm Maverik (Boston Scientific Scimed Inc, Natick, Mass.) over the wire balloon was advanced over a 0.014 inch guidewire (Guidant Corporation Temecula, Calif.) into the LAD after the first diagonal arterial branch. The coronary balloon was inflated incrementally, until complete occlusion was confirmed by angiography. A similar procedure was performed in the distal circumflex artery, proximal to the bifurcation of second obtuse marginal artery. The coronary balloon was inflated incrementally, until complete occlusion was confirmed by angiography. The AIV, and similarly the GCV at the entrance of the middle cardiac vein, were occluded during LAD and LCX delivery, respectively. With both the arterial and the venous balloons inflated (total 3 minutes), and following infusion of intracoronary adenosine (25 μg), gene transfer was performed by anterograde injection through the center lumen of the angioplasty balloon with adenoviral solution (1 ml of ˜10¹⁴ genomes in each coronary).

Three months following gene transfer of SERCA2a, there was a significant improvement in the parameters of contractility and complete reversal of heart failure, as shown in Table 5.

TABLE 5
Hemodynamic Parameters: AAV6-CMV-SERCA2a Gene Transfer

| | | | MR + | MR + |
|---|---|---|---|---|
| | Control | MR | AAV6βgal | AAV6SERCA2a |
| Heart rate (bpm) | 102 ± 8 | 156 ± 6 | 162 ± 18 | 126 ± 6 |
| Fractional shortening | 42.3 ± 4 | 33.0 ± 5 | 18.5 ± 6 | 37.3 ± 4 |
| (%) | | | | |
| Stroke volume (mL) | 34.2 ± 1.3 | 24.7 ± 2.1 | 19.2 ± 2.0 | 31.7 ± 2 |
| +dP/dt (mmHg/sec) | 1865 ± 324 | 1240 ± 245 | 1156 ± 412 | 1398 ± 344 |
| −dP/dt (mmHg/sec) | −1562 ± 388 | −1114 ± 191 | −1033 ± 422 | −1514 ± 101 |
| LV Systolic Pressure | 92 ± 5 | 89 ± 5 | 82 ± 4 | 98 ± 4 |
| (mmHg) | | | | |
| LV End-diastolic | 7 ± 2 | 14 ± 1 | 19 ± 4 | 10 ± 3 |
| Pressure (mmHg) | | | | |
| # of animals | 22 | 19 | 8 | 9 |

### Example 16

### Clinical Trial with AAV6.SERCA2a in Patients with End-Stage Heart Failure

An adeno-associated viral vector type 6 expressing SERCA2a driven by the cytomegaolvirus CMV promoter (AAV-CMV-SERCA2a) can be given by direct intracardiac injection to patients undergoing left ventricular assist device (LVAD), implantation. Based on the doses used in pigs during intracoronary injection (e.g., described in Examples 14 and 15) and in primates using direct injection of AAV2-CMV-sTNFR, two doses of AAV6-CMV-SERC2a can be given to patients undergoing LVAD placement for decompensated heart failure with non-ischemic cardiomyopathies (no coronary artery lesion ≧50%) as a bridge to transplant. The study can test, inter alia, whether: (a) the vector induces an inflammatory response, and (b) the expression of SERCA2a is persistent.

After placement of the LVAD and prior to the discontinuation of the bypass, two regions on the surface of the left ventricle can be identified, marked with sutures and then injected with AAV6-CMV-SERCA2a using a method identical to that used in the non-human primate studies. Each region can consist of a square with nine injection sites, with 1 cm between sites. Each site can be injected with 0.1 ml of virus-containing solution, 5 mm below the surface. One of the grids can be injected with a dose of 5×10¹² particles/ml and the other with a dose of 5×10¹¹ particles/ml. The two regions can be separated by at least 2 cm. The chest can then be closed according to standard procedures. Each patient can be followed between implantation and transplantation. Serum samples can be obtained weekly to measure CPK, troponin I, and troponin T, as markers of myocytolysis and/or toxicity. In addition, we can measure routine chemistries including BUN, Creatinine, liver function tests and hematologic profile, including sedimentation rate weekly. All participating LVAD patients can be treated according to standard protocols. Patients can be seen routinely by their physicians who can pay special attention to the development of fever, infection, arrhythmia or any unexpected finding that might be attributable to the gene therapy.

At the time of LVAD placement, the core tissue sample can be removed for further analysis. At the time of cardiac transplantation, the recipient's heart can be removed after cold cardioplegia. After placement in a cold cardioplegia solution, the injection sites can be identified and core samples obtained from each site. After dividing the samples into four equal pieces, two samples can be frozen in liquid nitrogen, one sample can be placed in formalin for histologic analysis, and one sample can be placed in OTC for subsequent immunohistochemical analysis. These samples can then be analyzed for SERCA2a expression, the presence or absence of inflammation, and routine histopathology. Samples can also be obtained from regions between the squares to assess the amount of regional spread of AAV expression.

Studies can be performed simultaneously on samples obtained from the core samples obtained at the time of LVAD placement, the two AAV-CMV-SERCA2a treatment sites, and the regions of the heart distant from the treatment sites. We can assess differences in histopathology (including cellular infiltrates, cell death, and cardiomyocyte width). The expression of genes defining cardiac function (SERCA2a, phospholamban, L-type Ca²⁺ channel Caᵥ1.3, sodium-calcium exchanger NCX, atrial natriuretic factor, α- and β-myosin heavy chain) can be assessed by both Northern and Western blot analyses. The expression of pro-inflammatory cytokines (TNFα, IL-1β, and IL-6) can be assessed at the protein and/or transcript level, using ELISA and RPA analyses. The expression of collagens can also be determined by Northern blot analysis of collagen proal (I) and (III) transcripts, total and soluble collagen, and immunohistochemical staining for type I and type III collagens. The primary comparisons can be made between injected and untreated areas of the heart; however, comparisons can also be made with the pre-LVAD myocardial sample. These comparisons can provide an important marker of the potentially beneficial changes that occur in the failing heart during LVAD support.

In addition to providing information about the effectiveness of SERCA2a in altering the cellular phenotype in LVAD-supported hearts, we can also gain important information regarding the safety of AAV vectors and SERCA2a overexpression in the human heart, as well as demonstrate the persistence of AAV-SERCA2a expression.

All patients can be recruited from those patients undergoing LVAD implantation. The major cause of death in these patients includes thromboembolic complications and device failure; however, the majority of these deaths occur within the first 30 days of LVAD implantation.

Postoperative cardiac function can be followed by serial echocardiography, and metabolic stress testing, according to the following timetable:
1. Echocardiography: Assessment of fractional area change to assess restoration of native cardiac function. This can be performed at 2 weeks, 4 weeks, 6 weeks, 8 weeks and 12 weeks post implantation. Each assessment can be initially performed on full VAD support. For individuals in whom fractional area change suggests an LVEF (left ventricular ejection fraction)>40% on VAD support, factional change can be reassessed during transient reduction of LVAD flow (as outlined in the weaning protocol).
2. Metabolic stress testing: Functional capacity on VAD can be assessed by stress testing, using measurement of respiratory gas exchange. This can be performed in all patients at 4 weeks, 8 weeks and 12 weeks post LVAD.

For subjects in whom routine screening suggests the potential for weaning from the device, the following protocol can be followed. Three months after LVAD implantation, each patient can undergo assessment as defined below. The protocol can have three steps: 1) assessment of cardiac function, using echocardiographically derived variables; 2) measurement of cardiac hemodynamics including cardiac output, left ventricular filling pressure, and pulmonary artery pressures and heart rate using a pulmonary artery catheter (Swann-Ganz catheter); and 3) assessment of functional capacity using an exercise stress test with measurements of respiratory gas exchange. Each of these pieces of information can provide an endpoint for this study; however, the utility of the specific intervention, i.e., SERCA2a therapy, can require demonstrating an ability to wean patients with a greater level of efficacy.

Prior to weaning, patients should meet the following criteria:
1) No less than 60 days of ventricular assist device support, or 90 days if a beta blocker or angiotensin converting enzyme inhibitor was initiated at or within one week of LVAD implantation.
2) Appropriate medical therapy, including an ACE inhibitor and beta-blocker, unless patient was intolerant of either of these two medications for reasons other than hemodynamic instability (eg., thrombocytopenia, angioedema, etc.).
3) Absence of arrhythmia.
4) Good nutritional status.
5) Participation in an in-hospital exercise program.
6) An echocardiogram demonstrating a left ventricular ejection fraction of >30%.
7) A symptom-limited exercise stress test using a Modified Bruce protocol and simultaneous respiratory gas exchange measurements demonstrating a peak VO2 of greater than 17.

Steps for Weaning:

1. VAD Flow Reduction.
The VAD flow can be reduced for a one-week period. Protocols for flow reduction are pump-dependent.
A) Novacor pump—the eject delay of the Novacor pump can gradually be increased, effectively reducing the LVAD support from 1:1 to 1:2 and eventually 1:3 of the native heart beats.
B) Thoratec pump—the Thoratec VAD rate can be increased gradually to 140 beats per minute over the seven days and the percent systole increased to 70%. This can effectively reduce the stroke volume of the pump to approximately 30 cc.
C) HeatMate VAD—the patient can first be converted from the electrical console to the pneumatic console, and again the effective pump rate can be reduced to trigger at only 1:3 for each native heart beat.

2. Assessment of Echocardiographic LV Function.

In patients who have tolerated one week of partial weaning, we can assess echocardiographic left ventricular function. While on LVAD support, patients LV function can be assessed during decreasing mechanical circulatory support and can include simultaneous measurements of: arterial blood pressure (peripheral), respiratory rate, electrocardiographic signals, and left ventricular cross sectional area (as a surrogate for volume). Echocardiogarphic automated border detection (Agilent Technologies, Sonos 5500) can be used from mid-ventricular short axis plane. All signals can be recorded on a computer using a customized analog/digital acquisition system (DATAQ Instruments, Akron, Ohio; model DI-220) and software (DATAQ Instruments; WINDAq vs. 1.92) for post-processing.

Physiologic data can be acquired at baseline with full LVAD support, followed by periods of decreases in LVAD support to 1:2 fixed rate setting. An initial bolus of heparin anticoagulation can be administered prior to the initiation of the study to minimize the risk of thrombus formation. Blood pressure and symptomatic assessment of the patient can be done throughout the protocol. The weaning protocol can be aborted if the patient becomes hypotensive (systolic BP<85 mmHg) or experiences signs of lightheadedness, dyspnea, or other symptoms of hypoperfusion. If the patient tolerates the initial decrease in pump rate, LVAD support can be further decreased as follows: Novascor systems (Baxter-Edwards) can be decreased to a minimum of 30 bpm, Heartmate systems (Thermocardiosystems) can be turned down to a minimum of 20 bpm, while Thoratec (Thoratec, Inc.) drivelines can be disconnected from the drive console and re-connected to hand bulbs. These hand bulbs can be compressed once every six seconds to prevent blood stasis and thrombus formation.

Analysis of the echocardiographic data can be performed using custom-written subroutines, calculating LV end diastolic area, end systolic area, fractional area change and systolic and diastolic pressures. Baseline values can be compared to parameters calculated from 1:2 fixed rate studies and off-pump (hand bulb) studies. All acquired data can then be analyzed using an analysis of variance for repeated measure.

3. Hemodynamic Assessment of Left Ventricular Function:

The patients who maintain a left ventricular ejection fraction of at least 40% during the echocardiographic assessment can be taken to the cardiac catheterization laboratory, where a pulmonary artery catheter can be placed under fluoroscopic control. Measurement can be made within one week of the echo measurements. With settings optimized as described for the echocardiographic protocol, right heart catheterization pressures can be measured, including cardiac output, left ventricular filling pressure, pulmonary artery pressure and heart rate. All hemodynamic measurements should be within the normal range.

4. Measurement of Functional Capacity.

Once echocardiographic function and pulmonary artery hemodynamics have been obtained, the VAD will be turned off and patients will again undergo symptom-limited exercise on a treadmill using a Modified Bruce Protocol and simultaneous pulmonary gas-exchange measurements. If patients maintain a VO2 max of greater than 15, they will be identified as being candidates for VAD explanation.

Similarly designed studies can be used to evaluate other methods of introducing viral delivery systems into the heart, e.g., methods of delivering such systems to the coronary arteries, and any other method described herein.

### Example 17

### Prevention of Ventricular Arrhythmias with SERCA2a Overexpression

Two common patterns in the initiation of fatal arrhythmias have been recognized in patients with ischemic heart disease: ventricular tachyarrhythmia, triggered by acute myocardial ischemia and during reperfusion in patients with or without preexisting myocardial scarring, and ventricular tachyarrhythmia, related to an anatomical scarring from previous myocardial infarctions without active myocardial ischemia.

At the molecular level, the mechanisms of ventricular arrhythmias are heterogeneous, but common mechanisms have been identified in triggering arrhythmias as changes in the membrane potential, ion transporters and intracellular Ca²⁺ handling.

Certain aspects of the role of abnormal Ca²⁺ signaling in the genesis of cardiac arrhythmias has been known for many years through various mechanisms. Ca²⁺ overload of the sarcoplasmic reticulum (SR) generate spontaneous release of Ca²⁺ by the Ryanodyne-Receptors and a depolarizing inward current mediated by sodium-calcium exchanger. These spontaneous events are known as delayed afterdepolarizations, and they underlie triggered arrhythmias. Early afterdepolarizations are another source of arrhythmias, occurring during reperfusion injury, caused by prolonged action potentials allowing excessive Ca²⁺ entry through L-type Ca²⁺ channels. Also, reperfusion-induced increase in Ca²⁺ concentration induces heterogeneity of repolarization. Abnormal Ca²⁺ cycling by the SR has been also implicated in the pathogenesis of action potential alternans and spiral wave break-up and ventricular fibrillation.

In this Example, whether SR Ca²⁺ ATPase pump (SERCA2a) overexpression could protect from ventricular arrhythmias by modulating Ca²⁺ overload was examined.

The following materials and methods were used in this Example.

### Materials and Methods

Animal Studies

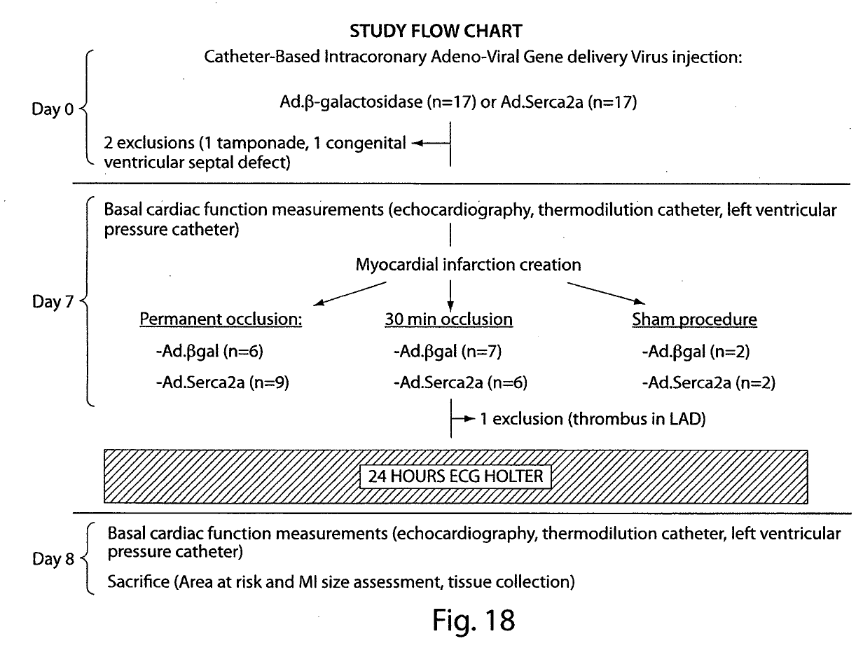

All procedures were performed under anesthesia (Isoflurane) and the Institutional Animal Care approved the experiment protocol. All pigs were female of the same body weight. In this set of experiments 34 pigs were first randomized to receive either the control adenovirus carrying β-galactosidase (n=17) or SERCA2a gene (n=17) (FIG. 18).

Construction of Adenovirus

The adenoviruses were constructed by the method described by He et al, which is incorporated herein by reference. SERCA2a cDNA was subcloned into the adenoviral shuttle vector (pAd.TRACK), which uses the cytomegalovirus (CMV) long terminal repeat as a promoter. The shuttle vector used also has a concomitant green fluorescent protein (GFP) under the control of a separate CMV promoter. An adenovirus containing both β-galactosidase and GFP controlled by separate CMV promoters (Ad. β-gal-GFP) was used as control.

Gene Delivery

The gene was delivered to the myocardium using percutaneous anterograde myocardial gene transfer (PAMGT) (Hayase M. et al.).

Using this technique, viral vectors were injected directly in the coronary artery distal to an angioplasty balloon occluding the vessel proximally while the venous drainage was occluded thought the coronary sinus. To occlude the venous drainage, via a right femoral approach, a 50 cm 8 8 F modified AL1 (Cordis Corporation, Miami, Fla.) was advanced to the coronary sinus, followed by a 110 cm 5 F wedge balloon (Allow International Inc, Reading, Pa.) over a guidewire. The balloon catheter was inflated until coronary venous occlusion was confirmed by angiography. For the percutaneous coronary arterial access a 7 F Hockey stick guiding catheter (Cordis Corporation, Miami, Fla.) was placed in the left coronary artery. A 6 mm length, 4.0 mm Sprinter (Medtronic, Inc; Minneapolis, Minn.) balloon was advanced over the wire, proximal site of left anterior descending artery (LAD), and the coronary balloon inflated incrementally until complete occlusion was confirmed by angiography. Similarly, an angioplasty balloon was placed proximal to the left circumflex and right coronary arteries. For all the LAD, left circumflex coronary artery and the right coronary artery territories, the myocardium was preconditioned with a 1-minute arterial balloon occlusion. With both the arterial and venous balloons inflated (total 3 minutes), and following an intracoronary adenosine (25 μg) injection to increase cellular permeability, PAMGT was performed by antegrograde injection through the lumen of the angioplasty balloon with either an adenoviral solution (1 ml of 10¹¹ plaque-forming units in each coronary) carrying β-galactosidase or SERCA2a. Arterial blood pressure was continuously monitored.

Procedures Protocol

Seven days following gene delivery, pigs from each group (SERCA2a and β-galactosidase) were assigned to undergo ischemia-reperfusion (I/R) using 30 min balloon occlusion (n=13) or permanent occlusion (PO) using embolic coils (n=16) in the LAD or a sham procedure without LAD occlusion (n=4) (FIG. 18). Aortic pressure, EKG and oxygen blood saturation were monitored throughout any procedure. An EKG Holter device was connected to the pigs at the beginning of I/R, PO or sham procedure. Echocardiography and invasive hemodynamic parameters were measured at baseline before I/R, PO or sham procedure.

Twenty-four hours later, the Holter recording was stopped and the device removed for analysis. The echocardiography parameters and the invasive hemodynamic parameters were repeated at this time point.

Echocardiography

Transthoracic 2-dimensional and M-Mode echocardiography images were obtained in anesthetized animals with a 3.4 MHz probe (General Electric, Vivid7). A mid-papillary level left ventrical (LV) short-axis view was used to measure anterior wall thickness, LV systolic and diastolic dimension, and fractional shortening.

Thermodilution Catheter

A Swan-Ganz catheter was inserted through the femoral vein to measure the pressure in the right atria, right ventricle, pulmonary artery and pulmonary capillary wedge. The catheter was then positioned in the pulmonary artery to measure cardiac output by thermodilution method.

Left Ventricular Pressure Measurement

A Millar pigtail catheter was introduced through the femoral artery in the LV cavity. Pressure measurements were digitized at 1 KHz. LV systolic pressure (LVSP), LV end-diastolic pressure (LVEDP), the maximal rates of pressure rise (+dP/dt) and of pressure fall (−dP/dt) were measured off line.

Arrhythmia Recording

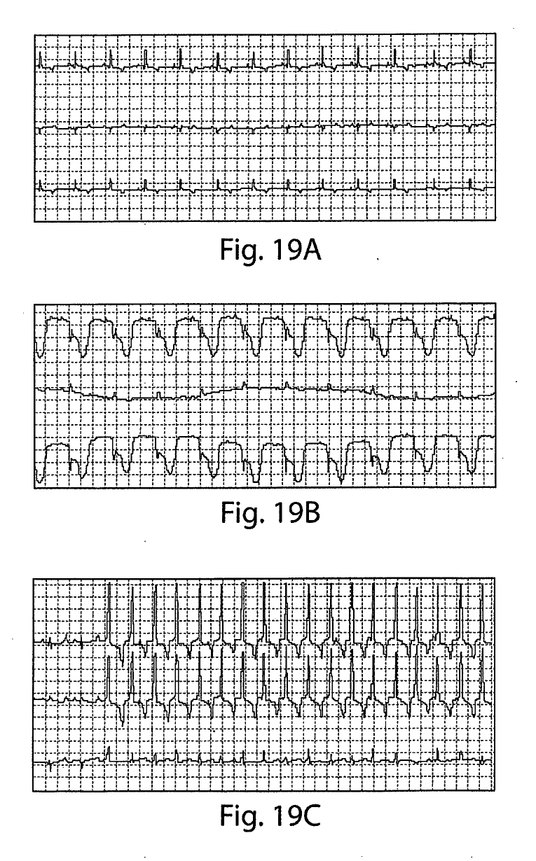

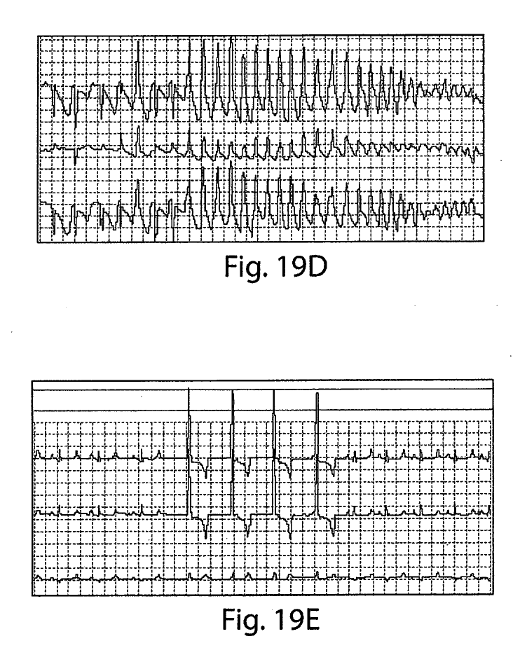

The Holter device (Del Mar Reynolds, Spacelabs Healthcare, Issaquah, Wash.) was attached to the pigs using a protective bandage around the chest to record the EKG over 24-h period. Three adhesive leads were placed at the fifth intercostal space on the left and right anterior axillary line with the reference electrode on the manubrium of the sternum. EKG was continuously recorded (FIG. 19).

In order to classify ventricular arrhythmias, 3 periods of time in the I/R groups were defined: (1) period of ischemia corresponding to 30 minutes of LAD occlusion (ischemia); (2) reperfusion period corresponding to the first 10 minutes immediately after balloon deflation (early reperfusion); (3) follow-up period corresponding to the time from the eleventh minute after balloon deflation to the end of the Holter recording, i.e., 24 hours later or to the last ventricular beat in case of death (late reperfusion). In the PO groups, the follow-up period started from the occurrence of ST elevation, approximately 1 minute after coil insertion. EKG was continuously monitored in all groups for 90 min from the balloon inflation or coil insertion. Sustained ventricular tachycardia (VT) or ventricular fibrillation (VF) were treated by defibrillation during these 90 minutes. VT was defined by more than 3 consecutive ventricular extra beats with heart rate superior or equal to 120 beat per minutes; Sustained VT was defined by VT duration over 30 seconds.

Ischemia-Reperfusion

Seven days after gene delivery in the I/R groups a coronary balloon was inflated in mean LAD beyond the takeoff of the first diagonal branch for 30 min, inducing transmural ischemia and then deflated to reperfuse the LAD territory. In details, LAD was cannulated with a 7 F Hockey Stick guiding catheter, 100 μg nitroglycerine injected, and baseline coronary angiography performed. A 3.5-mm over-the-wire balloon catheter was deployed in the LAD beyond the takeoff of the first diagonal branch to induce transmural ischemia. Coronary angiography was performed to confirm total occlusion with the balloon. After 30 min occlusion, the balloon catheter was deflated to reperfuse the LAD. The duration of the ischemic time was previously shown to the critical for the induction of arrhythmia upon reperfusion with an occlusion time below two minutes and above 45 minutes no longer inducing the vulnerability to ventricular fibrillation. The duration of the occlusion time is also determinant to the incidence of reperfusion arrhythmias as function of the extent of the ischemic injury arguing for the importance of the release of metabolic products from the injured myocytes during the occlusion time. We therefore limited the occlusion time to 30 min. Continuous EKG and aortic pressure were carefully monitored during the procedure.

Permanent Coronary Occlusion

As described for the I/R groups the LAD was cannulated with a 7 F Hockey Stick guiding catheter, 100 μg nitroglycerine injected, and baseline coronary angiography performed. An embolic coil (0.018 inch, 4 cm length 4 mm×2 mm diameter, Cook) was introduced with a 2.6 French micro catheter (Excelsior, Boston Scientific/Target, Fremont, Calif.) into the LAD beyond the takeoff of the first diagonal branch to completely occlude the mid AD and induce myocardial infarction. Coronary angiography was performed to confirm the total occlusion with the coil.

Gene Distribution

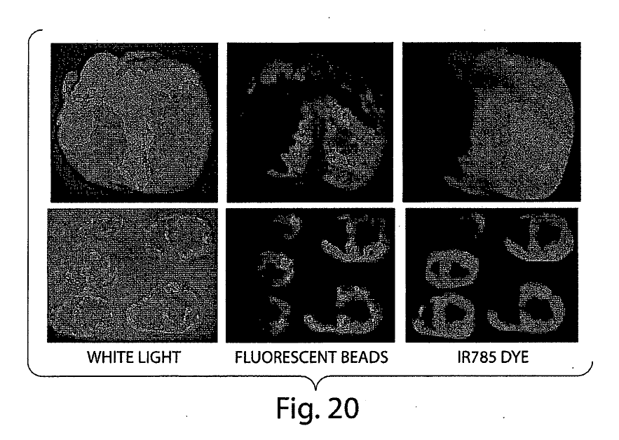

Intracoronary injection was performed in a spare pig to visualize the distribution area by injecting a near-infrared fluorescent dye (IRDYE 786) (Sigma-Aldrich No. 102185-03-5) and fluorescent microspheres (Molecular Probes) instead of adenoviral solution (FIG. 20).

Detection of Gene Expression by Immunohistochemistry

Gene infection was detected by expression of the reporter gene β-galactosidase on frozen sections. The staining is based on the hydrolysis of X-gal (5-bromo-4-chloro-3-indoyl β-D-galactopyranoside), which yields a blue precipitate. β-galactosidase activity was measured using X-gal 40 mg/ml in dimethylformamide on tissue sections fixed with 0.5% glutarhaldehyde.

Immunoblotting

Protein concentration of tissue lysate preparations were measured using the Bradford method. Immunoblotting were performed under reducing condition on a gradient gel. For immunoreactions, the blots were incubated with antibodies for anti-SERCA2a (Ab made in the laboratory); anti-GADPH (Zymed, 39-8600, Zymed Laboratories, Inc., California) was used for normalization against total proteins.

RNA Isolation and Retrotranscription

The levels of human SERCA2a present in the anterior wall samples from four animals were quantified for SERCA2a and β-galactosidase I/R groups and one sample from a sham pig by reverse transcriptase PCR (qRT-PCR). 100 mg of tissue were homogenized in TRIzol (Invitrogen, CA) and processed according to the manufacturer guidelines.

RNA was retrotranscribed using Omniseript RT Kit (Qiagen, Valencia, Calif.) following the manufacturer protocol. qRT-PCR was performed on the cDNA obtained from the reverse transcription using RT² SYRR Green/ROX PCR Master Mix (Superscript, MD) using the following conditions: 15 min at 95° C., 40 cycles (95° C. 15 sec, 64° C. 30 sec, 72° C. 30 sec). The following primers were used to evaluate the content of human SERCA2a var2 mRNA:

| | |
|---|---|
| Forward: | 5′-CCTCCCACAAGTCTAAAATC-3′ |
| Reverse: | 5′-AGCAATGCCAATCTCGGCT-3′ |

In order to differentiate the endogenous SERCA2a from the exogenous human SERCA2a injected in the tissue, the human vs. the swine sequences of SERCA2a were compared and the regions characterized by the higher inter-specific variability were identified. These primers were manually selected to interact with these regions. Primer3 software (http://frodo.wi.mit.edu/cgi-bin/primer3/primer3_www.cgi) was used to identify the primer combinations characterized by lower self-dimerization and 3′ compatibility.

Because of the high conservation of the nucleotide sequence of SERCA2a, the choice of primers was limited and the primers selected were characterized respectively by 2 and 5 nucleotide mismatches between the two sequences. As expected from the design process, the selected primers were affected by high 3′ complementarities, producing a background in the blank. The combination of the use of these primers with elevated annealing temperature produced a negligible amplification of the swine SERCA2a DNA.

The content of the transcript in each sample was standardized to the level of swine I8S RNA using the following primers:

| | |
|---|---|
| Forward: | 5′-AGACAAATCGCTCCACCAAC-3′ |
| Reverse: | 5′-GACTCAACACGGGAAACCTC-3′ |

The data were analyzed with 7000 SDS 1.1 RQ application (Applied Biosystem, CA) and the software automatically detected the CT threshold for both the transcripts analyzed.

Area at Risk and Myocardial Infarction Size Assessment

In order to assess the area at risk (AAR), a solution with red fluorescent microspheres (Molecular Probes) mixed to near-infrared fluorescent dye (IRDYE 786) (Sigma-Aldrich #102185-03-5) was injected immediately before sacrifice into the proximal coronary arteries while a balloon was inflated at the same location than during ischemia in all reperfused pigs and without any balloon in all pigs with PO or sham procedure. To delineate the infarct size, the hearts were sliced in 1 cm thick slices and stained with triphenyltetrazolium chloride (TTC, Sigma) as previously described. The slices were imaged under a NIR fluorescent camera to identify the distribution of the beads and dye into the myocardium. The AAR and infarct area were measured from digital micrographs with NIH Image. The AAR was defined by the area delineated by the absence of the microspheres. Percentage of myocardial infarction was calculated as the total infarcted area, unstained by TTC, divided by the total AAR for the heart.

Statistical Analysis

All results are expressed as mean±standard deviation. Between-group differences were compared by Student's t-test or variance for continuous variables, and chi-square tests for categorical variables. A comparison was considered significant when p≦0.05.

### Results

Three pigs were excluded due to procedural complications. One pig was excluded due to tamponade at the time of gene delivery; at autopsy, a vein anatomy variation was found. One pig was excluded due to a congenital ventricular septal defect identified at the time of gene delivery. One last pig was excluded at the time of balloon occlusion due to the formation of a thrombus occluding the LCX.

Cardiac Function

As expected at baseline, echocardiography and invasive hemodynamic data were similar in all groups (Table 1).

TABLE 1
Baseline echocardiography and hemodynamic data

| | β-Gal-Coil | SERCA2a-Coil | β-Gal-I/R | SERCA2a-I/R | β-Gal-Sham | SERCA2a-Sham |
|---|---|---|---|---|---|---|
| | (n = 6) | (n = 9) | (n = 6) | (n = 6) | (n = 2) | (n = 2) |
| Baseline morphological data | | | | | | |
| BW, kg | 42.1 ± 2.8 | 42.3 ± 11.0 | 42.8 ± 8.6 | 51.6 ± 2.3 | 40.5 ± 2.1 | 47.5 ± 3.5 |
| Baseline echocardiography data | | | | | | |
| AWd, cm | 0.89 ± 0.13 | 0.68 ± 0.18 | 0.76 ± 0.11 | 0.87 ± 0.12 | 0.84 ± 0.01 | 0.82 ± 0.04 |
| AWs, cm | 1.15 ± 0.19 | 1.29 ± 0.15 | 1.16 ± 0.11 | 1.33 ± 0.06 | 1.11 ± 1.17 | 1.27 ± 0.19 |
| LVEDD, cm | 4.30 ± 0.73 | 4.53 ± 0.35 | 4.43 ± 0.02 | 4.21 ± 0.15 | 3.92 ± 0.50 | 4.62 ± 0.52 |
| LVESD, cm | 2.70 ± 0.48 | 2.84 ± 0.27 | 2.66 ± 0.09 | 2.46 ± 0.17 | 2.36 ± 0.35 | 2.69 ± 0.36 |
| FS, % | 37.21 ± 2.56 | 38.77 ± 1.58 | 39.92 ± 2.11 | 41.79 ± 3.39 | 39.54 ± 2.17 | 42.03 ± 1.41 |
| Baseline invasive hemodynamic | | | | | | |
| data | | | | | | |
| HR, bpm | 80 ± 16 | 93 ± 7 | 89 ± 10 | 91 ± 17 | 100 ± 7 | 100 ± 15 |
| SAP, mmHg | 89 ± 9 | 100 ± 11 | 106 ± 17 | 89 ± 6 | 111 ± 18 | 92 ± 3 |
| CO, L/min | 5.33 ± 0.64 | 5.65 ± 0.56 | 5.58 ± 0.95 | 5.00 ± 0.65 | 5.56 ± 0.13 | 5.46 ± 0.11 |
| LVEDP, mmHg | 5.83 ± 2.32 | 8.11 ± 2.57 | 6.93 ± 1.73 | 5.33 ± 2.34 | 7.00 ± 2.83 | 4.00 ± 1.41 |
| dP/dt, mmHg · s⁻¹ | 1236 ± 164 | 1385 ± 229 | 1393 ± 258 | 1323 ± 140 | 1728 ± 609 | 1315 ± 274 |
| −dP/dt, mmHg · s⁻¹ | 1408 ± 115 | 1568 ± 167 | 1669 ± 423 | 1432 ± 143 | 1666 ± 35 | 1415 ± 105 |

β-Gal indicates pigs receiving β-galactosidase gene;
SERCA2a, pigs receiving SERCA2a gene;
Coil, permanent coronary artery occlusion;
BW, body weight;
AW, anterior wall thickness in diastole (d) and systole (s);
LVEDD, LV end-diastolic diameter;
LVESD, LV end-systolic diameter;
FS, fractional shortening;
HR, heart rate;
SAP, systolic aortic pressure;
CO, cardiac output;
LVEDP, LV end-diastolic pressure: and
dP/dt, first derivative of LV pressure. Values are mean ± SD.

Twenty-four hours following I/R, PO or sham procedure the echocardiography data, expectedly, showed significant left ventricular systolic function impairment in the PO groups as compared to the I/R groups. Similarly, the hemodynamic data showed that both PO groups exhibited significantly impaired systolic and diastolic parameters at higher filling pressure as compared to the I/R groups. The small amount of muscle necrosis involved in the I/R groups did not significantly impact left ventricular size and function as compared to sham group. In both models SERCA2a gene transfer did not affect significantly left ventricular hemodynamic and morphological data (Table 2).

TABLE 2
Final echocardiography and hemodynamic data

| | β-Gal-Coil | SERCA2a-Coil | β-Gal-I/R | SERCA2a-I/R | β-Gal-Sham | SERCA2a-Sham |
|---|---|---|---|---|---|---|
| | (n = 5) | (n = 5) | (n = 6) | (n = 6) | (n = 2) | (n = 2) |
| Final echocardiography data | | | | | | |
| AWd, cm | 0.78 ± 0.13 | 0.72 ± 0.06 | 0.81 ± 0.08 | 0.98 ± 0.12 | 0.81 ± 0.04 | 0.83 ± 0.04 |
| AWs, cm | 0.96 ± 0.11 | 1.03 ± 0.18 | 1.27 ± 0.16* | 1.33 ± 0.22* | 1.27 ± 1.16* | 1.32 ± 0.11* |
| LVEDD, cm | 4.77 ± 0.65 | 4.72 ± 0.13 | 4.54 ± 0.30 | 4.36 ± 0.34 | 4.44 ± 0.19 | 4.76 ± 0.32 |
| LVESD, cm | 3.57 ± 0.74 | 3.51 ± 0.19 | 2.97 ± 0.24† | 2.68 ± 0.31*† | 2.60 ± 0.01† | 2.86 ± 0.11† |
| FS, % | 25.53 ± 7.44 | 25.52 ± 2.30 | 34.56 ± 1.38*† | 38.65 ± 4.70*† | 40.53 ± 3.50*†‡ | 39.73 ± 1.80*†‡ |
| Final invasive hemodynamic | | | | | | |
| data | | | | | | |
| HR, bpm | 101 ± 10 | 94 ± 20 | 88 ± 13 | 83 ± 15 | 89 ± 4 | 87 ± 1 |
| SAP, mmHg | 81 ± 7 | 84 ± 9 | 93 ± 15 | 89 ± 6 | 83.5 ± 6 | 91 ± 4 |
| CO, L/min | 3.83 ± 0.97 | 3.40 ± 0.59 | 5.19 ± 1.20† | 4.68 ± 0.58† | 5.35 ± 0.18† | 5.40 ± 1.26† |
| LVEDP, mmHg | 14.25 ± 2.22 | 14.40 ± 1.82 | 8.88 ± 3.54*† | 7.30 ± 2.74*† | 10.25 ± 1.06† | 8.50 ± 2.12*† |
| dP/dt, mmHg · s⁻¹ | 965 ± 122 | 870 ± 157 | 1222 ± 236† | 1131 ± 195† | 1281 ± 126*† | 1157 ± 59 |
| −dP/dt, mmHg · s⁻¹ | 1086 ± 147 | 1020 ± 126 | 1356 ± 264† | 1416 ± 108*† | 1728 ± 229*† | 1446 ± 210† |

Abbreviations as in Table 1. Values are mean ± SD.
*P < 0.05 vs β-Gal-Coil;
†P < 0.05 vs SERCA2a-Coil;
‡P < 0.05 vs β-Gal-I/R.

Incidence of Ventricular Arrhythmias

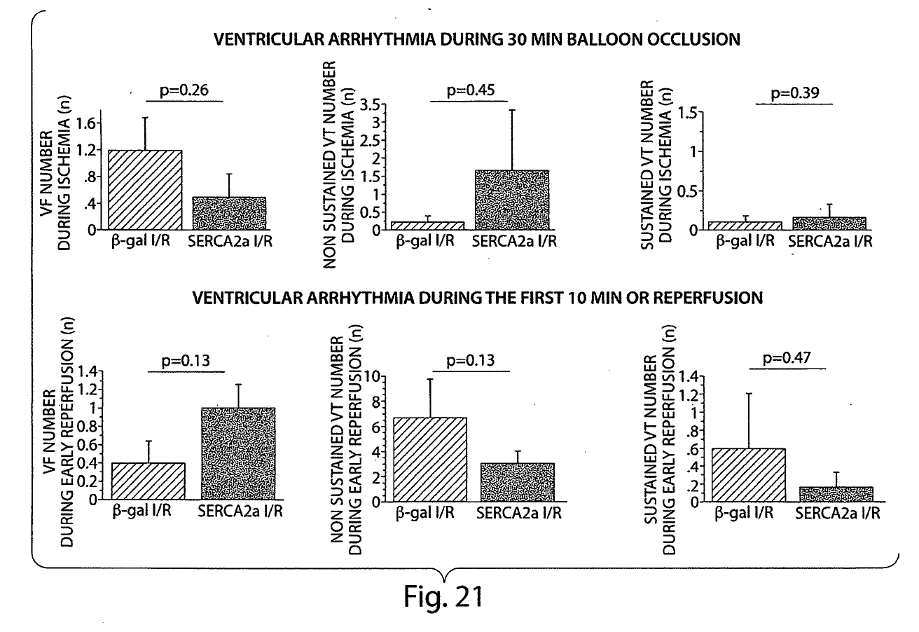

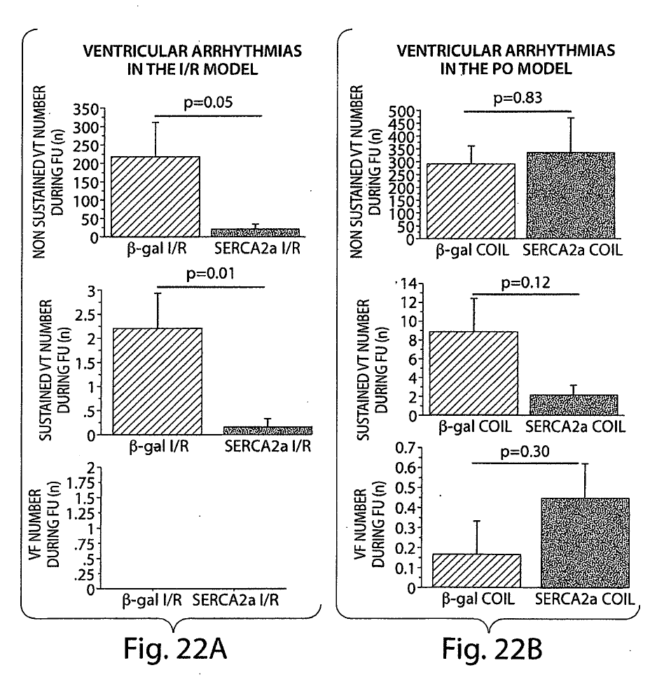

In the I/R groups, there was no significant difference between pigs overexpressing SERCA2a and β-galactosidase in VT or VF episodes during ischemia (FIG. 21). Similarly to the data obtained in a model of I/R in rats, SERCA2a overexpression significantly reduced life threatening arrhythmias after reperfusion, i.e., the total number of episodes of VF and VT that occurred from balloon deflation to the end of the follow-up (27±11 episodes in pigs overexpressing SERCA2a vs. 226±95 episodes in pigs overexpressing β-galactosidase, p=0.047). Few life threatening arrhythmias occurred in the early phase of the reperfusion (3±1 episodes in pigs overexpressing SERCA2a vs. 7±3 episodes in pigs overexpressing β-galactosidase, p=0.22), therefore antiarrythmic effect observed in pig overexpressing SERCA2a mainly occurred in the late phase of reperfusion (23±12 episodes in pigs overexpressing SERCA2a vs. 219±95 episodes in pigs overexpressing p-galactosidase, p=0.05). Detailed results of sustained and unsustained VT and VF episodes are presented in FIGS. 21 and 22. No episode of VF was detected in the late reperfusion period in the I/R groups neither with SERCA2a or the control virus (FIG. 22A).

Similarly to the data obtained in rats with permanent LAD occlusion, pigs with PO overexpressing SERCA2a exhibited a tendency towards an increase of fatal arrhythmias: 5 out of 9 pigs overexpressing SERCA2a (55.6%) and 2 out of 6 pigs overexpressing β-galactosidase (33.3%) required cardiac defibrillation during the first 90 min after coil insertion (p=0.40): 4 pigs overexpressing SERCA2a (44.4%) and 1 pig overexpressing β-galactosidase (16.7%) died because of VF event during the follow-up (p=0.26). These pigs with late fatal VF following PO were not all the same that have been saved by defibrillation in the first 90 min after coil insertion (2 out of 4 in pigs overexpressing SERCA2a and 0 out of 1 in pigs overexpressing β-galactosidase). There was no significant difference in the occurrence of sustained VT or non sustained VT and VF episodes between both PO) groups although a tendency towards the reduction of the sustained VI′ and increase in VF was observed with SERCA2a overexpression (FIG. 22B).

Gene Expression

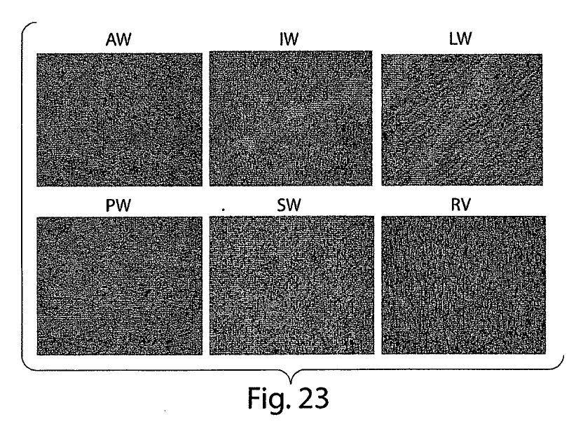

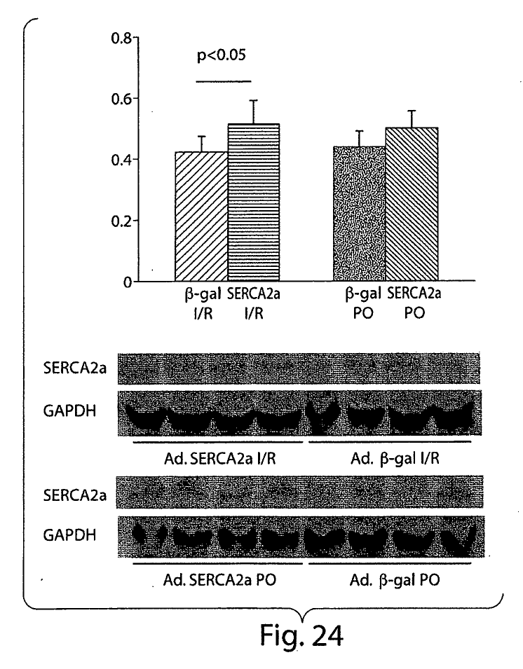

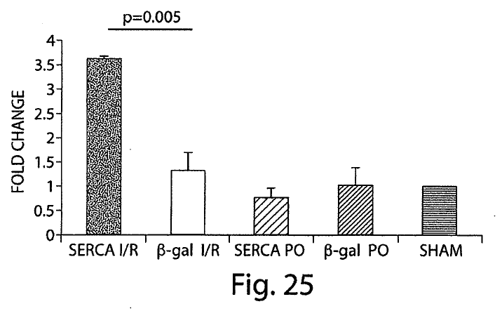

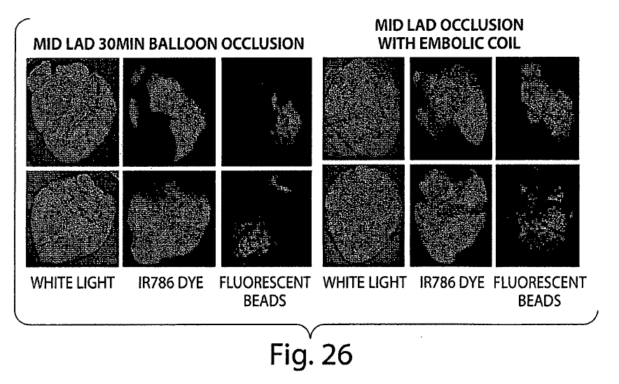

The areas of gene distribution identified by the near infrared fluorescent dye (IRDYE 786) (Sigma-Aldrich #102185-03-5) and fluorescent microspheres (Molecular Probes®) exhibited homogeneous distribution of the particles in the left ventricle (FIG. 20). The expression efficiency was demonstrated by immunohistochemistry, immunoblotting and qRT-PCR. The distribution of the blue β-gal expression was homogeneous across the ventricular walls following the expression of the control protein (FIG. 23). The expression of the SERCA2a protein and the level of human SERCA2a variant gene by qRT-PCR showed increased SERCA2a expression compared to controls although the difference was statistically significant only in the IR group (FIGS. 24 and 25). It is possible that sampling in the necrotic area would account for the finding.

Quantification of the Ischemic and Necrotic Area

The AAR was approximately 30% of the left ventricle in PO and reperfusion groups (Table 3) and was similar in the β-galactosidase and in the SERCA2a group showing consistency in the level of occlusion among animals (FIG. 7).

TABLE 3
AAR and infarction size data

| | β-Gal-Coil | SERCA2a-Coil | β-Gal-I/R | SERCA2a-I/R |
|---|---|---|---|---|
| | (n = 5) | (n = 25) | (n = 6) | (n = 6) |
| AAR/LV, | 26.0 ± 7.6 | 32.1 ± 5.7 | 33.5 ± 2.7 | 31.9 ± 3.3 |
| % | | | | |
| MI/AAR, | 97.6 ± 1.3 | 94.1 ± 9.5 | 20.3 ± 22.4*† | 0.7 ± 1.3*†‡ |
| % | | | | |

MI indicates myocardial infarction. Other abbreviations as in Table 1.
Values are mean ± SD.
*P < 0.001 vs β-Gal-Coil;
†P < 0.001 vs SERCA2a-Coil;
‡P = 0.057 vs β-Gal-I/R.

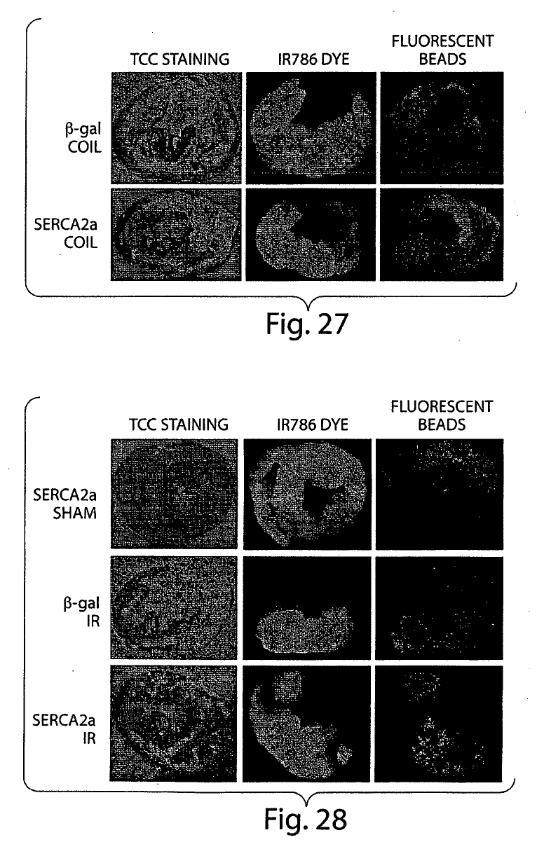

In the PO groups no difference in the infarct size was observed between β-galactosidase and SERCA2a groups (FIG. 27A, Table 3). Similarly to the data obtained in a model of IR in rats. SERCA2a overexpression induced reduction of the infarcted area in pigs undergoing I/R. (FIG. 27A, Table 3).

### Discussion

The inventors have shown that targeted gene transfer of SERCA2a to failing myocardium results in sustained improvement in ventricular function. Over the years, experience with pharmacotherapy has shown that agents that increase inotropy in diseased myocardium increase morbidity in terms of decreased survival and increased ventricular arrhythmias. Unlike pharmacological inotropic agents SERCA2a overexpression was whether increasing SR Ca²⁺ load would lead to oscillatory Ca²⁺ overexpression was associated with improved release from the SR and worsening arrhythmias. In a rat model in which Ca²⁺ overload was induced by ischemia followed by reperfusion, but not in a model of P0, it was shown that SERCA2a overexpression was able to protect from ventricular arrhythmias. The model of reperfusion injury compared to a model of PO allow us to investigate Ca²⁺ overload as a critical mechanisms for electrical instability in the ischemic and failing myocardium.

In the present study ventricular arrhythmias were abrogated in pigs overexpressing SERCA2a following induction of I/R, but SERCA2a failed to abrogate ventricular arrhythmias occurring in pigs with PO of the LAD as well as in the ischemic phase in both group.

Those data support the notion that Ca²⁺-overload to the surviving cells plays a key role in the origin of reperfusion arrhythmia and SERCA2a overexpression only protects from ventricular arrhythmia when the electrical instability is related to Ca²⁺-overload occurring upon reperfusion.

During ischemia, damage to the sarcolemmal membrane leads to increased influx of Ca²⁺ that worsens upon reperfusion due to the higher Ca²⁺ content of the catabolic products of the reperfusing blood flow. The increase in Ca²⁺ ion to the cell in turns overloads the SR allowing spontaneous Ca²⁺ leakage from the SR generating a depolarizing inward current with asynchronous spontaneous mechanical activity and afterdepolarizations. Also, agents that increase cAMP such as catecholamine can induce afterdepolarizations and aftercontractions and cAMP dependent protein kinase activity removes phospholamban inhibition increasing SERCA2a activity. Thus, the inventors hypothesized that SERCA2a overexpression would increase afterdepolarization-induced arrhythmias.

On the other hand, SERCA2a favoring sequestration of Ca²⁺ by the SR and a larger SR Ca²⁺ store will initially lead to an increase in Ca²⁺ transient. Autoregulation results from a more rapid inactivation of subsequent Ca²⁺ currents and reduced Ca²⁺ entry through L-type Ca²⁺ channels. The net effect would be to reduce transarcolemmal Ca²⁺ flux while maintaining a normal systolic transient. Thus, it might be expected that SERCA2a overexpression to reduce L-type current, recapitulating the effects of Ca²⁺ channel blockade on arrhytrmas. Also, SERCA2a, by reducing Ca²⁺ transient duration, was shown to reduce the occurrence of aftercontractions and afterdepolarization in isolated rabbit cardiomyocytes. The resequestration of Ca²⁺ in the SR compartment was confirmed by the increase in SR Ca²⁺ content calculated from the Na⁺/Ca²⁺-exchanger current evoked by rapid caffeine application.

Also Ca²⁺ dissociation from the myofilaments will initiate Ca²⁺ waves and triggered propagated contractions (TPC), DADs and triggered arrhythmic activity. SERCA2a may reduce the occurrence of afterdepolarization restoring intracellular Ca²⁺ homeostasis this reducing Ca²⁺ waves.

The effects of ischemia and heart failure on myocardial Ca²⁺ transient also include beat to beat Ca²⁺ transient alternans showed on the EKG as ST segment and T wave morphology alternans changes occurring in ischemia just before the onset of ventricular fibrillation. ST and T alternans have been attributed to the spatial and temporal heterogeneity in the APD in the myocardium, but heterogeneity of Ca²⁺ concentration within myocytes can also be involved. SERCA2a, by restoring Ca²⁺ reuptake and intracellular Ca²⁺ homeostasis, may reduce the Ca²⁺ transient alternans and ST-T alternants, triggering arrhythmia.

Furthermore a mechanism for SERCA2a protection from ventricular arrhythmias can reside in the protection from mitochondrial permeability transition pore that compromises ATP production following Ca²⁺ overload at reperfusion²⁵⁻²⁷. ATP depletion, in addition to increased intracellular Na⁺ and damage to Ca²⁺ handling proteins, can contribute to Ca²⁺ oscillation and increased [Ca²⁺]i that is taken up by mitochondria leading to further depletion of ATP contributing to ventricular arrhythmias. Improvement of SR Ca²⁺ handling can mediate protection from reperfusion injury through metabolic preservation.

It was also shown by this study that SERCA2a in a rat model and in a pig model of I/R reduces myocardial injury. SERCA2a may exert protection towards ventricular arrhythmia by reducing myocardial scarring from preservation of viable myocytes following ischemic injury. A previous study by the present inventors suggests a protective effect of SERCA2a on myocardial viability as SERCA2a gene expression significantly reduce the loss of viable rabbit myocytes over a 48 hrs culture. In addition, improving SR Ca²⁺ handling by protecting from mitochondrial Ca²⁺ overload may also prevent necrotic cell death by preserving ATP production.

On the other hand cardiac function is deteriorated in both permanent coronary artery occlusion groups. The degree of this impairment is about the same with or without SERCA2a overexpression. The lack of better cardiac function outcome after permanent occlusion in SERCA2a overexpressing pigs reflects the inability of SERCA2a overexpression to reduce MI necrosis in the context of a definitive coronary occlusion.

In conclusion, abnormal Ca²⁺ cycling and Ca²⁺ overload are critical in the induction and perpetuation of cardiac arrhythmia in acute and chronic conditions by mechanisms that can be targeted by favoring Ca²⁺ reuptake. SERCA2a by reducing Ca²⁺ overload, improves mechanical and electrical stability of the heart and may be a successful therapeutic approach that addresses the subcellular events critical for the initiation and perpetuating of arrhythmias.

### REFERENCES

1. Shah M, Akar F G, Tomaselli G F. Molecular basis of arrhythmias. Circulation. 2005; 112 (16):2517-2529.
2. Tomaselli G E, Rose J. Molecular aspects of arrhythmias associated with cardiomyopathies. Curr Opin Cardiol. 2000; 15 (3):202-208.
3. Liu X S, Jiang M, Zhang M, Tang D, Clemo H F, Higgins R S. Tseng G N. Electrical remodeling in a canine model of ischemic cardiomyopathy. Am J Physial Heart Circ Physiol. 2007; 292 (1):H560-571.
4. Armoundas A. A. Wu R, Juang G, Marban E, Tomaselli G F. Electrical and structural remodeling of the failing ventricle. Pharmacol Ther. 2001; 92 (2-3):213-230.
5. Fallavollita J A. Jacob S, Young R F, Canty J M, Jr. Regional alterations in SR Ca.(2+)-ATPase, phospholamban, and HSP-70 expression in chronic hibernating myocardium. Am .1 Physiol. 1999; 277 (4 Pt 2):1H418-1428.
6. Verrier R L. Hagestad El. Mechanisms involved in reperfusion arrhythmias. Eur Heart J. 1986; 7 Suppl A: 13-22.
7. Priori S G, Corr P B, Mechanisms underlying early and delayed afterdepolarizations induced by catecholamines. Am J Phy.siol. 1990; 258 (6 Pt 2):H1796-1805.
8. Zhao S X, Lee L M, Nearing B D, Busso V O, Kwaku K E, Verrier R I. Suppression of calcium-induced repolarization heterogeneity as a mechanism of nitroglycerin's antiarrhythmic action. J Cardiovasc Pharmacol. 2006; 48 (2):22-29.
9. Brown A M. Excitation of afferent cardiac sympathetic nerve fibres during myocardial ischaemia. J Physiol. 1967; 190 (1) 35-53.
10. He T C, Zhou S, da Costa L T, Yu J, Kinzler K W, Vogelstein B. A simplified system for generating recombinant adenoviruses. Proc Natl Acad Sci USA. 1998; 95 (5):2509-2514.
11. Hayase M. Del Monte F, Kawase Y, Macneill B D, McGregor J, Yoneyama R, Hoshino K, TsujiDe Grand A M, Gwathmey J K, Frangioni J V, Hajjar R J. Catheter-based antegrade intracoronary viral gene delivery with coronary venous blockade. Am J Physiol Heart Circ Physiol. 2005:288 (6):H2995-3000.
12. Balke C W, Kaplinsky E, Michelson E L, Naito M, Dreifus L S. Reperfusion ventricular tachyarrhythmias: correlation with antecedent coronary artery occlusion tachyarrhythmias and duration of myocardial ischemia. Am Heart J. 1981; 10 I (4):449-456.
13. Sewell W H, Koth D R., Huggins C E. Ventricular fibrillation in dogs after sudden return or flow to the coronary artery. Surgery. 1955; 38 (6):1050-1053.
14. Corbalan R, Verrier R L, Lown B. Differing mechanisms for ventricular vulnerability during coronary artery occlusion and release. Am Heart J. 1976; 92 (2):223-230.
15. Verrier R L, Nearing B D. Ambulatory ECG monitoring of T-wave alternans for arrhythmia risk assessment. J Electrocanliol. 200336 Suppl:193-197.
16. Flaumenhaft R. Tanaka F. Graham G J, Dc Grand A M, Laurence 14 (R G, Hoshino K, Hajjar, R J Frangioni J V. Localization and quantification of platelet-rich thrombi in large blood vessels with near-infrared fluorescence imaging. Circulation 2007:115 (1):84-93.
17. Bradford M. Protein measurement. Anal. Biochem. 1976; 72:248-260.
18. del Monte F. Lebeche O, Guerrero J L, Tsuji T, Doye A A, Gwathmey J K, Hajjar R J. Abrogation of ventricular arrhythmias in a model of ischemia and reperfusion by targeting myocardial calcium cycling. Proc Natl Acad Sci US.A. 2004; 101 (15):5622-5627.
19. Chen Y, Escoubet 13, Prunier F, Amour J, Simonides W S, Vivien B, Lenoir C, Heimburger M, Choqueux C, Gellen Riou B, Michel 113, Franz W M, Mercadier J J. Constitutive cardiac overexpression of sareoplasmic/endoplasmic reticulum Ca²⁺-ATPase delays myocardial failure after myocardial infarction in rats at a cost of increased acute arrhythmias. Circulation. 2004; 109 (15): 1898-1903.
20. del Monte F, Williams F. Lebeche D, Schmidt U, Rosenzweig A, Gwathmey J K, Lewandowski E D, Hajjar Improvement in survival and cardiac metabolism after gene transfer of sarcoplasmic reticulum Ca(2+)-ATPase in a rat model of heart failure. Circulation. 2001; 104 (12):1424-1429.
21. Miura M, Boyden P A, ter Keurs H E. Ca2+ waves during triggered propagated contractions in intact traheculae. Am J Physiol. 1998; 274 (1 Pt 2):H266-276.
22. Terracciano C M, Tweedie D, MacLeod K T. The effects of changes to action potential duration on the calcium content of the sarcoplasmie reticulum in isolated guinea-pig ventricular myocytes. Pflugers Arch. 1997/A33 (4):542-544.
23. Davia K, Bernobich E, Ranu H K, del Monte F, Terracciano C M, MacLeod K T, Adamson D L, Chaudhri B, Hajjar R J, Harding S F. SERCA2A overexpression decreases the incidence of aftercontractions in adult rabbit ventricular myocytes. J Mol Cell Cardiol. 2001; 33 (5): 1005-1015.
24. Verrier R L, Nearing B D. Electrophysiologic basis For T wave alternans as an index of vulnerability to ventricular fibrillation. J Cardiovasc Electrophysiol. 1994:5 (5):445-46 1.
25. Javadov S A, Clarke S, Das M, Griffiths E J, Lim K U, Halestrap A P. Isehaernie preconditioning inhibits opening of mitochondrial permeability transition pores in Me reperfused rat heart. J Physiol. 2003; 549 (Pt 2):513-524.
26. Hausenloy D, Wynne A, Duchen M, Yellon D. Transient mitochondrial permeability transition pore opening mediates preconditioning-induced protection. Circulation. 2004; 109 (14):1714-1717.
27. Murphy E, Steenbergen C. Mechanisms underlying acute protection from cardiac ischemia-reperfusion injury. Physiol Rev, 2008; 88 (2):581-609.

### EQUIVALENTS

Those skilled in the art will recognize, or be able to ascertain, using no more than routine experimentation, many equivalents to the specific embodiments of the invention described herein. Such equivalents are intended to be encompassed by the following claims:

What is claimed is:

1. A method for treating arrhythmia in a subject with ischemic heart disease, comprising enhancing the function of SERCA2a, thereby treating the arrhythmia in the subject.

2. The method of claim 1, wherein enhancing the function of SERCA2a is achieved by administering to the subject a therapeutically effective amount of a recombinant expression vector encoding SERCA2a.

3. The method of claim 2, wherein the administration is local administration.

4. The method of claim 2, wherein the SERCA2a expression vector is administered to the heart of the subject by introducing the SERCA2a expression vector into one or more coronary vessels.

5. The method of claim 2, wherein the SERCA2a expression vector is administered to the heart of the subject by introducing the SERCA2a expression vector into one or more coronary vessels by percutaneous anterograde myocardial gene transfer.

6. The method of claim 1, wherein enhancing the function of SERCA2a is achieved by administering a therapeutically effective amount of an agent that increases SERCA2a function.

7. The method of claim 6, wherein the agent that increases SERCA2a function is selected from the group consisting of a compound that increases the activity of SERCA2a, a compound that decreases the activity of phospholamban, or an inhibitory RNA of phospholamban.

8. The method of claim 1, wherein the arrhythmia is a reperfusion-induced arrhythmia.

9. The method of claim 1, wherein the arrhythmia is a ventricular tachyarrhythmia.

10. The method of claim 9, wherein the tachyarrhythmia is triggered by acute myocardial ischemia.

11. The method of claim 9, wherein the tachyarrhythmia is triggered by reperfusion.

12. The method of claim 1, wherein the arrhythmia is a ventricular tachyarrhythmia which results from a previously formed myocardial infarction scar and wherein the subject is without active myocardial ischemia.

13. The method of claim 1, wherein the arrhythmia is a tachyarrhythmia or bradyarrythmia.

14. The method of claim 1, wherein the arrhythmia is of an atrial, junctional, atro-ventricular or ventricular origin.

15. The method of claim 1, wherein the vector is a DNA or RNA viral vector.

16. The method of claim 15, wherein the vector is an adenovirus, adeno-associated virus, or lentivirus vector.

17. The method of claim 16, wherein the vector is AAV-6.

18. The method of claim 5, wherein the agent is administered using a stent.

19. A method for protecting against tissue damage or death caused by reperfusion-induced arrhythmia in a subject with acute myocardial ischemia comprising enhancing the function of SERCA2a, thereby protecting against tissue damage or death.

20. The method of claim 19, wherein enhancing the function of SERCA2a is achieved by administering to the heart of the subject at the time of reperfusion or prior to onset of the acute myocardial ischemia, a therapeutically effective amount of a recombinant expression vector encoding SERCA2a.

21. The method of claim 20, wherein the SERCA2a expression vector is administered to the heart of the subject by introducing the SERCA2a expression vector into one or more coronary vessels.

22. The method of claim 21, wherein the administering is local administration.

23. The method of claim 20, wherein the SERCA2a expression vector is administered to the heart of the subject by introducing the SERCA2a expression vector into one or more coronary vessels by percutaneous anterograde myocardial gene transfer.

24. The method of claim 20, wherein the SERCA2a expression vector is administered to the heart of the subject by introducing the SERCA2a expression vector directly into the myocardium.

25. The method of claim 20, wherein the step of enhancing the function of SERCA2a is achieved by administering a therapeutically effective amount of an agent that increases SERCA2a function.

26. The method of claim 25, wherein the agent that enhances SERCA2a function is selected from the group consisting of a compound that enhances the activity of SERCA2a, a compound that decreases the activity of phospholamban, or an inhibitory RNA of phospholamban.

27. The method of claim 19, wherein the reperfusion-induced arrhythmia is a ventricular tachyarrhythmia or ventricular bradyarrythmia.

28. The method of claim 19, wherein the reperfusion-induced arrhythmia is of an atrial, junctional, atro-ventricular or ventricular origin.

29. The method of claim 19, wherein the recombinant expression vector is an adenovirus, adeno-associated virus, or lentivirus vector.

30. The method of claim 29, wherein the recombinant expression vector is AAV-6.

31. The method of claim 25, wherein the agent is administered using a stent.

32. A method for reducing risk of death or injury due to arrhythmia in a subject with ischemic heart disease, comprising:
occluding a coronary artery and a coronary vein with a first and second angioplasty balloon, respectively, thereby restricting the flow of blood through the coronary vessels;
injecting through the lumen of the first angioplasty balloon into the lumen of the coronary artery a recombinant expression vector encoding SERCA2a, thereby causing the vector to perfuse the myocardium, wherein the vector thereafter expresses the SERCA2a in the myocardium, thereby reducing risk of death or injury due to arrhythmia in a subject with ischemic heart disease.

33. The method of claim 32, wherein the arrhythmia is a reperfusion-induced arrhythmia.

34. The method of claim 32, wherein the arrhythmia is a ventricular tachyarrhythmia.

35. The method of claim 34, wherein the tachyarrhythmia is triggered by acute myocardial ischemia.

36. The method of claim 34, wherein the tachyarrhythmia is triggered by reperfusion.

37. The method of claim 32, wherein the arrhythmia is a tachyarrhythmia or bradyarrythmia.

38. The method of claim 32, wherein the arrhythmia is of an atrial, junctional, atro-ventricular or ventricular origin.

39. The method of claim 32, wherein the recombinant expression vector is an adenovirus, an adeno-associated virus, or a lentivirus vector.

40. The method of claim 39, wherein the recombinant expression vector is AAV-6.

41. The method of claim 32, wherein the recombinant expression vector comprises a myocardial-specific promoter that is operably linked to a nucleic acid encoding SERCA2a.

42. The method of claim 32, wherein the coronary artery is the left anterior descending artery (LAD) or the distal circumflex artery (LCX), and coronary vein is the great coronary vein (GCV), the middle cardiac vein (MCV), or the anterior interventricular vein (AIV).

43. The method of claim 32, wherein the step of injecting the vector through the lumen occurs after ischemic preconditioning of the coronary vessels.

44. The method of claim 32, wherein the step restricting the flow through the coronary vessels is for a period of about 30 seconds to 5 minutes.

45. The method of claim 43, wherein the step of ischemic preconditioning is for a period of about 1 to 4 minutes.

46. The method of claim 32, wherein the subject is a human.
